(19) United States
(12) Patent Application Publication
(10) Pub. No.: US 2015/0093399 A1
(43) Pub. Date: Apr. 2, 2015

(54) CNS-TARGETED CONJUGATES HAVING MODIFIED FC REGIONS AND METHODS OF USE THEREOF

(71) Applicant: biOasis Technologies, Inc., Richmond (CA)

(72) Inventor: Wilfred Jefferies, South Surrey (CA)

(21) Appl. No.: 14/472,186

(22) Filed: Aug. 28, 2014

Related U.S. Application Data
(60) Provisional application No. 61/870,914, filed on Aug. 28, 2013.

(51) Int. Cl. A61K 47/48; A61K 38/17

(52) U.S. Cl. CPC A61K 47/483; A61K 38/1793
USPC 424/178.1; 530/391.7

(57) ABSTRACT

Provided are central nervous system (CNS)-targeted antibody or therapeutic Fc-fusion polypeptide conjugates having modified Fc regions, and related methods of use thereof, for instance, to facilitate delivery of therapeutic and/or diagnostic polypeptides across the blood-brain barrier (BBB), and thereby treat and/or diagnose conditions associated with the CNS, including cancer, pain, and various neuropathologies, such as neuroinflammatory, auto-immune, and/or neurodegenerative disorders.

### CROSS-REFERENCE TO RELATED APPLICATIONS

This application claims priority under 35 U.S.C. §119(e) to U.S. Application No. 61/870,914, filed Aug. 28, 2013, which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is BIOA_{—}007_{—}01 US_ST25.txt. The text file is about 88 KB, was created on Aug. 28, 2014, and is being submitted electronically via EFS-Web.

### BACKGROUND

1. Technical Field

The present invention relates to central nervous system (CNS)-targeted antibody or therapeutic Fc-fusion polypeptide conjugates having modified Fc regions, and related methods of use thereof, for instance, to facilitate delivery of therapeutic and/or diagnostic polypeptides across the blood-brain barrier (BBB), and thereby treat and/or diagnose conditions associated with the CNS, including cancer, pain, and various neuropathologies, such as neuroinflammatory, auto-immune, and/or neurodegenerative disorders.

2. Description of the Related Art

Overcoming the difficulties of delivering therapeutic or diagnostic agents to specific regions of the brain represents a major challenge to treatment or diagnosis of many central nervous system (CNS) disorders, including those of the brain. In its neuroprotective role, the blood-brain barrier (BBB) functions to hinder the delivery of many potentially important diagnostic and therapeutic agents to the brain.

Therapeutic molecules and genes that might otherwise be effective in diagnosis and therapy do not cross the BBB in adequate amounts. It is reported that over 95% of all therapeutic molecules do not cross the blood-brain barrier.

Accordingly, there is a need for compositions and methods that facilitate the delivery of therapeutic agents and other molecules across the blood-brain-barrier, for instance, to effectively treat certain diseases of the central nervous system (CNS) such as cancers, particularly those that have metastasized to the CNS. The present invention addresses these needs and offers other related advantages.

### BRIEF SUMMARY OF THE INVENTION

Embodiments of the present invention include conjugates, comprising a blood-brain barrier (BBB)-transport moiety linked to an antibody or therapeutic Fc-fusion polypeptide, where the Fc region of the antibody or therapeutic Fc-fusion polypeptide is modified, for instance, to alter its binding to (or interaction with) one or more Fc receptors or other soluble or cell-associated binding partners. Examples of such modifications include amino acid substitutions within the Fc region and full or partial deletions of the Fc region. In specific instances, the Fc region of an antibody is nearly or fully deleted, to generate, for example, a conjugate that comprises a BBB-transport moiety linked to a Fab fragment or F(ab′)₂ fragment.

According to one non-limiting theory, the Fc region of an antibody or therapeutic Fc-fusion polypeptide-based conjugate interacts with Fc receptor(s) or Fc ligand(s) and competes with (i.e., reduces) the interaction between the BBB-transport moiety and its ligand(s), thereby altering distribution of the conjugate in a way that reduces its transport across the BBB and delivery to tissues of the central nervous system (CNS). Accordingly, the CNS-targeting of antibody or Fc-fusion polypeptide-based conjugates can be improved by generating Fc region modifications which alter (e.g., reduce) the interaction between the Fc region and one or more Fc receptors or ligand(s).

Embodiments of the present invention therefore include conjugates, comprising a blood-brain barrier (BBB)-transport moiety linked to a therapeutic antibody, wherein the Fc region of the antibody is modified to reduce binding to one or more Fc receptors/ligands. In certain embodiments, the antibody specifically binds to one or more of human Her2/neu, Her1/EGFR, TNF-α, B7H3 antigen, CD20, VEGF, CD52, CD33, CTLA-4, tenascin, alpha-4 (α4) integrin, IL-23, amyloid-β such as Aβ₍₁₋₄₂₎, Huntingtin, CD25, nerve growth factor (NGF), TrkA, or α-synuclein.

In some embodiments, the antibody specifically binds to a cancer-associated antigen. In particular embodiments, the cancer-associated antigen is one or more of human Her2/neu, Her1/EGF receptor (EGFR), Her3, A33 antigen, B7H3, CD5, CD19, CD20, CD22, CD23 (IgE Receptor), C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αᵥβ₃, integrin α₅β₁, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), or mesothelin.

In some embodiments, the antibody specifically binds to a pro-inflammatory molecule, optionally a pro-inflammatory cytokine or chemokine. In certain embodiments, the pro-inflammatory molecule is one or more of TNF-α, TNF-β, FasL, CD27L, CD30L, CD40L, Ox40L, 4-1BBL, TRAIL, TWEAK, and Apo3L, IL-1α, IL-1β, IL-2, interferon-γ (IFN-γ), IFN-α, IFN-β, IL-6, IL-8, IL-12, IL-15, IL-17, IL-18, IL-21, LIF, CCL5, GROα, MCP-1, MIP-1α, MIP-1β, macrophage colony stimulating factor (MCSF), or granulocyte macrophage colony stimulating factor (GM-CSF). In certain embodiments, the pro-inflammatory molecule is TNF-α, and where the antibody is adalimumab, certolizumab pegol, golimumab, infliximab, D2E7, CDP 571, or CDP 870.

In some embodiments, the antibody specifically binds to a pain-associated antigen. In certain embodiments, the pain associated-antigen is one or more of nerve growth factor (NGF) or tropomyosin-related kinase A (TrkA).

In certain embodiments, the therapeutic antibody is selected from trastuzumab, cetuximab, rituximab, daclizumab, tanezumab, 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, cantuzumab (mertansine), cantuzumab (ravtansine), capromab (pendetide), catumaxomab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, tucotuzumab (celmoleukin), ublituximab, urelumab, veltuzumab, volociximab, votumumab, and zalutumumab.

Also included are conjugates, comprising a blood-brain barrier (BBB)-transport moiety linked to a therapeutic Fc-fusion polypeptide, where the Fc region of the Fc-fusion polypeptide is modified to reduce the binding to one or more Fc receptors/ligands. In some embodiments, the Fc-fusion polypeptide is selected from etanercept, abatercept, aflibercept, alefacept, belatacept, rilonacept, and romiplastin.

In certain embodiments, the Fc region is modified to reducing binding to one or more of a protein of the complement system, Fcγ receptors (FcγR), Fcα receptors (FcαR), Fcε receptors (FcεR), or the neonatal Fc receptor (FcRn), relative to a corresponding unmodified Fc region. In some embodiments, the protein of the complement system is C1, optionally the C1q subunit thereof. In certain embodiments, the FcγR is selected from one or more of FcγRI, FcγRIIa, FcγRIIb, FcγRIIc, FcγRIIIa, and FcγRIIIb. In certain embodiments, the FcαR is selected from one or both of FcαRI (CD89) or Fcα/μR. In some embodiments, the FcεR receptor is selected from one or both of FcεRI and FcεRII.

In some embodiments, the Fc region is modified to reduce one or more effector functions selected from complement fixation or activation, complement-dependent cytotoxicity (CDC)-related activity, antibody-dependent cellular cytotoxicity (ADCC)-related activity, and/or antibody-dependent cell phagocytosis (ADCP)-related activity, relative to a corresponding unmodified Fc region. In some embodiments, the Fc region is modified by full or partial deletion of the Fc region, optionally including a full or partial deletion of the hinge region. In certain embodiments, the Fc region is modified by full or partial deletion of one or more of the CH₂ region, CH₃ region, CH₄ region, and/or hinge region. In some embodiments, the Fc region is modified by full or partial deletion of the C1q binding site. In some embodiments, the Fc region of the antibody is deleted to generate a Fab fragment or a F(ab′)₂ fragment of the antibody.

In certain embodiments, the BBB-transport moiety is selected from one or more of a p97 (melanotransferrin) polypeptide, a Receptor Associated Protein (RAP), an aprotinin peptide or an analog thereof, a protein transduction domain (PTD), a human low-density lipoprotein receptor (hLDLR) binding peptide or an analog thereof, an antibody or natural ligand that binds to a BBB-associated receptor, and glutathione (GSH).

In some embodiments, the p97 polypeptide comprises or consists of a sequence in Table B1, or an active variant or fragment thereof. In certain embodiments, the p97 polypeptide is a soluble human p97 polypeptide. In certain embodiments, the RAP comprises or consists of a sequence in Table B2, or an active variant or fragment thereof.

In some embodiments, the aprotinin peptide comprises or consists of a sequence in Table B3, or an active variant or fragment thereof.

In some embodiments, the PTD comprises or consists of a sequence in Table B4, or an active variant or fragment.

In some embodiments, the hLDLR binding peptide comprises or consists of a sequence in Table B5, or an active variant or fragment.

In certain embodiments, the BBB-associated receptor is selected from one or more of the insulin receptor, the transferrin receptor, the leptin receptor, lipoprotein receptors such as the lipoprotein receptor-related protein (LRP-1) receptor, insulin-like growth factor (IGF) receptors such as IGF1R and IGF2R, the low-density lipoprotein receptor, the diptheria toxin receptor, and TMEM 30A (Flippase).

In certain embodiments, the BBB-associated receptor ligand is selected from one or more of insulin, transferrin and transferrin fragments, lactoferrin and lactoferrin fragments, apolipoprotein A (Apo A), apolipoprotein B (Apo B), apolipoprotein E (Apo E), and diptheria toxin (including non-toxic mutants thereof such as CRM45 and CRM197).

Also included are compositions, comprising a pharmaceutically acceptable carrier or excipient, and a conjugate described herein.

Some embodiments include methods of treating a subject in need thereof, comprising administering to the subject a composition/conjugate described herein.

Certain embodiments include methods of delivering a therapeutic antibody or Fc-fusion protein to the central nervous system of subject in need thereof, comprising administering to the subject a composition/conjugate described herein.

Certain embodiments include methods for treating a cancer of the central nervous system (CNS), optionally the brain.

Also included are methods for treating primary cancer of the CNS, optionally the brain. Some embodiments include methods for treating a metastatic cancer of the CNS, optionally the brain. Some methods are for treating a glioma, meningioma, pituitary adenoma, vestibular schwannoma, primary CNS lymphoma, neuroblastoma, or primitive neuroectodermal tumor (medulloblastoma). In certain embodiments, the glioma is an astrocytoma, oligodendroglioma, ependymoma, or a choroid plexus papilloma.

Also included are methods for treating glioblastoma multiforme. In some embodiments, the glioblastoma multiforme is a giant cell gliobastoma or a gliosarcoma.

Also included are methods for treating a degenerative or autoimmune disorder of the central nervous system (CNS). In certain embodiments, the degenerative or autoimmune disorder of the CNS is Alzheimer's disease, Huntington's disease, Parkinson's disease, or multiple sclerosis (MS).

Certain methods are for treating pain. In certain embodiments, the pain is acute pain, chronic pain, neuropathic pain, and/or central pain.

Also included are methods for treating an inflammatory condition. In some embodiments, the inflammatory condition has a central nervous system component. In some embodiments, the inflammatory condition is one or more of meningitis, myelitis, encephalomyelitis, arachnoiditis, sarcoidosis, granuloma, drug-induced inflammation, Alzheimer's disease, stroke, HIV-dementia, encephalitis, parasitic infection, an inflammatory demyelinating disorder, a CD8+ T Cell-mediated autoimmune disease of the CNS, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, stroke, traumatic brain injury (TBI), spinal stenosis, acute spinal cord injury, and spinal cord compression.

In certain embodiments, the inflammatory condition is associated with an infection of the central nervous system. In some embodiments, the infection is a bacterial infection caused by one or more of group B streptococci (e.g., subtypes III), Streptococcus pneumoniae (e.g., serotypes 6, 9, 14, 18 and 23), Escherichia coli (e.g., carrying K1 antigen), Listeria monocytogenes (e.g., serotype IVb), neisserial infection such as Neisseria meningitidis (meningococcus), staphylococcal infection, heamophilus infection such as Haemophilus influenzae type B, Klebsiella, Mycobacterium tuberculosis, Treponema pallidum, or Borrelia burgdorferi. In certain embodiments, the infection is a viral infection caused by one or more of an enterovirus, herpes simplex virus type 1 or 2, human T-lymphotrophic virus, varicella zoster virus, mumps virus, human immunodeficiency virus (HIV), or lymphocytic choriomeningitis virus (LCMV).

In certain embodiments, the inflammatory condition is associated with a cancer of the CNS, optionally a malignant meningitis.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE FIGURES

FIG. 1 shows the volume fractions of MTf (p97), etanercept, and MTf-etanercept conjugate present in the brain capillaries and brain parenchyma at 2, 6, and 24 hours post-injection.

FIG. 2 shows the volume fractions of MTf (p97), Fab, and MTf-Fab conjugate present in the brain capillaries and brain parenchyma at 2, 6, and 24 hours post-injection.

FIG. 3 shows the synthesis route for MTf conjugates (see Example 1).

### DETAILED DESCRIPTION OF THE INVENTION

Embodiments of the present invention are based partly on the theory that central nervous system (CNS)-targeting of antibody or Fc-fusion polypeptide-based conjugates, which comprise a BBB-transport moiety, can be improved by Fc region modifications which alter (e.g., reduce) the interaction between the Fc region and one or more Fc receptors or Fc ligands. Such Fc modifications are expected to enhance the ability of the BBB-transport moiety to facilitate delivery of the conjugate across the BBB, for instance, by minimizing the interactions between the Fc region and its ligands and thereby favoring the interaction between the BBB-transport moiety of the conjugate and its BBB-associated receptor(s) or ligand(s).

Hence, embodiments of the present invention include conjugates, comprising a blood-brain barrier (BBB)-transport moiety linked to an antibody or therapeutic Fc-fusion polypeptide, wherein the Fc region of the antibody or Fc-fusion polypeptide is modified to reduce binding to one or more Fc ligands, compositions that comprise such conjugates, and related methods of use, including methods of treatment, diagnosis, and testing, such as medical imaging.

In particular embodiments, the methods relate to the treatment and/or diagnosis of various neuropathologies, including neurodegenerative and/or auto-immune-based neuropathologies, cancers of the CNS, and pain, including acute, chronic, and neuropathic pain, among others.

The conjugates described herein can thus find a variety of uses in the therapeutic and diagnostic arts, for instance, to improve transfer of therapeutic/diagnostic antibodies and existing Fc-fusion polypeptides across the BBB and targeting to tissues of the nervous system, including the CNS. Other advantages and benefits will be apparent to persons skilled in the art.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “conjugate” is intended to refer to the entity formed as a result of covalent or non-covalent attachment or linkage of an agent or other molecule, e.g., a biologically active molecule, to a BBB-transport moiety, such as a p97 (melanotransferrin) polypeptide. One example of a conjugate polypeptide is a “fusion protein” or “fusion polypeptide,” that is, a polypeptide that is created through the joining of two or more coding sequences, which originally coded for separate polypeptides; translation of the joined coding sequences results in a single, fusion polypeptide, typically with functional properties derived from each of the separate polypeptides.

As used herein, the terms “function” and “functional” and the like refer to a biological, enzymatic, or therapeutic function.

“Homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The term “linkage,” “linker,” “linker moiety,” or “1” is used herein to refer to a linker that can be used to separate a BBB-transport moiety from an agent of interest, or to separate a first agent from another agent, for instance where two or more agents are linked to form a CNS-targeted or BBB-targeted conjugate. The linker may be physiologically stable or may include a releasable linker such as an enzymatically degradable linker (e.g., proteolytically cleavable linkers). In certain aspects, the linker may be a peptide linker, for instance, as part of a fusion protein. In some aspects, the linker may be a non-peptide linker or non-proteinaceous linker. In some aspects, the linker may be particle, such as a nanoparticle.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between. As one non-limiting example, a control could compare the activity, such as the amount or rate of transport/delivery across the blood brain barrier, the rate and/or levels of distribution to central nervous system tissue, and/or the Cₘₐₓ for plasma, central nervous system tissues, or any other systemic or peripheral non-central nervous system tissues, of a CNS-targeted or BBB-targeted conjugate relative to the unconjugated agent (e.g., antibody, Fc-fusion polypeptide), or of a CNS-targeted, Fc-modified conjugate relative to a corresponding CNS-targeted conjugate having no Fc modification(s) or different Fc modification(s). Other examples of comparisons and “statistically significant” amounts are described herein.

In certain embodiments, the “purity” of any given agent (e.g., a conjugate such as a fusion protein) in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to: carboxylate ester, phosphate ester, anhydride, acetal, ketal, acyloxyalkyl ether, imine, orthoester, thio ester, thiol ester, carbonate, and hydrazone, peptides and oligonucleotides.

A “releasable linker” includes, but is not limited to, a physiologically cleavable linker and an enzymatically degradable linker. Thus, a “releasable linker” is a linker that may undergo either spontaneous hydrolysis, or cleavage by some other mechanism (e.g., enzyme-catalyzed, acid-catalyzed, base-catalyzed, and so forth) under physiological conditions. For example, a “releasable linker” can involve an elimination reaction that has a base abstraction of a proton, (e.g., an ionizable hydrogen atom, Ha), as the driving force. For purposes herein, a “releasable linker” is synonymous with a “degradable linker.” An “enzymatically degradable linkage” includes a linkage, e.g., amino acid sequence that is subject to degradation by one or more enzymes, e.g., peptidases or proteases. In particular embodiments, a releasable linker has a half life at pH 7.4, 25° C., e.g., a physiological pH, human body temperature (e.g., in vivo), of about 30 minutes, about 1 hour, about 2 hour, about 3 hours, about 4 hours, about 5 hours, about 6 hours, about 12 hours, about 18 hours, about 24 hours, about 36 hours, about 48 hours, about 72 hours, or about 96 hours or less.

The term “reference sequence” refers generally to a nucleic acid coding sequence, or amino acid sequence, to which another sequence is being compared. All polypeptide and polynucleotide sequences described herein are included as references sequences, including those described by name and those described in the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., “Current Protocols in Molecular Biology,” John Wiley & Sons Inc, 1994-1998, Chapter 15.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “solubility” refers to the property of a polypeptide or conjugate to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (˜37° C.). In certain embodiments, a CNS-targeted polypeptide or conjugate has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/ml at room temperature or at about 37° C.

A “subject,” as used herein, includes any animal that exhibits a symptom, or is at risk for exhibiting a symptom, which can be treated or diagnosed with a CNS-targeted conjugate of the invention. Suitable subjects (patients) include laboratory animals (such as mouse, rat, rabbit, or guinea pig), farm animals, and domestic animals or pets (such as a cat or dog). Non-human primates and, preferably, human patients, are included.

“Substantially” or “essentially” means nearly totally or completely, for instance, 95%, 96%, 97%, 98%, 99% or greater of some given quantity.

“Substantially free” refers to the nearly complete or complete absence of a given quantity for instance, less than about 10%, 5%, 4%, 3%, 2%, 1%, 0.5% or less of some given quantity. For example, certain compositions may be “substantially free” of cell proteins, membranes, nucleic acids, endotoxins, or other contaminants.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

BBB-Transport Moieties

The conjugates described herein comprise a “blood-brain barrier (BBB)-transport moiety,” or a component that increases delivery of the conjugated agent across the BBB relative to the agent alone. The BBB-transport moiety can covalently, non-covalently, or operatively coupled to the agent of interest, such as a therapeutic antibody, therapeutic Fc-fusion polypeptide), and/or a diagnostic or detectable agent, to form a CNS-targeted conjugate. In some embodiments, the BBB-transport moiety is a polypeptide or peptide sequence. Exemplary BBB-transport peptide moieties or sequences are described below, and include p97 (melanotransferrin) polypeptides, aprotinin-based peptide sequences, and others. See, for example, Gabathuler, Neurobiology of Disease. 37:48-57, 2010; and Gabathuler, Therapeutic Delivery. 1:571-586, 2010.

p97 Sequences.

In certain embodiments, the BBB-transport moiety is a p97 (melanotransferrin) polypeptide or sequence. In some embodiments, a p97 polypeptide sequence used in a composition and/or conjugate of the invention comprises, consists essentially of, or consists of the human p97 melanotransferrin sequence set forth in SEQ ID NO:1. Also included are variants and fragments thereof.

In some embodiments, a p97 polypeptide sequence comprises a sequence having at least 70%, 75%, 80%, 85%, 90%, 95%, 96%, 97%, 98%, or 99% identity or homology, along its length, to the human p97 sequence set forth in SEQ ID NO:1, or a portion thereof.

In particular embodiments, a p97 polypeptide sequence comprises a fragment of a human p97 sequence set forth in SEQ ID NO:1. In certain embodiments, a p97 polypeptide fragment is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700. 700, 710, 720, 730 or more amino acids in length, including all integers and ranges in between, and which may comprise all or a portion of the sequence of a reference p97 sequence such as SEQ ID NO:1.

In certain embodiments, a p97 polypeptide fragment is about 5-700, 5-600, 5-500, 5-400, 5-300, 5-200, 5-100, 5-50, 5-40, 5-30, 5-25, 5-20, 5-15, 5-10, 10-700, 10-600, 10-500, 10-400, 10-300, 10-200, 10-100, 10-50, 10-40, 10-30, 10-25, 10-20, 10-15, 20-700, 20-600, 20-500, 20-400, 20-300, 20-200, 20-100, 20-50, 20-40, 20-30, 20-25, 30-700, 30-600, 30-500, 30-400, 30-300, 30-200, 30-100, 30-50, 30-40, 40-700, 40-600, 40-500, 40-400, 40-300, 40-200, 40-100, 40-50, 50-700, 50-600, 50-500, 50-400, 50-300, 50-200, 50-100, 60-700, 60-600, 60-500, 60-400, 60-300, 60-200, 60-100, 60-70, 70-700, 70-600, 70-500, 70-400, 70-300, 70-200, 70-100, 70-80, 80-700, 80-600, 80-500, 80-400, 80-300, 80-200, 80-100, 80-90, 90-700, 90-600, 90-500, 90-400, 90-300, 90-200, 90-100, 100-700, 100-600, 100-500, 100-400, 100-300, 100-250, 100-200, 100-150, 200-700, 200-600, 200-500, 200-400, 200-300, or 200-250 amino acids in length, and comprises all or a portion of a reference p97 sequence such as SEQ ID NO:1.

In certain embodiments, p97 polypeptide sequences of interest include p97 amino acid sequences, subsequences, and/or variants of p97 that are effective for transporting an agent of interest across the blood brain barrier and into the central nervous system (CNS). In particular embodiments, the variant or fragment comprises the N-lobe of human p97 (residues 20-361 of SEQ ID NO:1). In specific aspects, the variant or fragment comprises an intact and functional Fe³⁺-binding site.

In some embodiments, a p97 polypeptide sequence is a soluble form of a p97 polypeptide (see Yang et al., Prot Exp Purif. 34:28-48, 2004), or a fragment or variant thereof. In some aspects, the soluble p97 polypeptide has a deletion of the all or a portion of the hydrophobic domain (residues 710-738 of SEQ ID NO:1), alone or in combination with a deletion of all or a portion of the signal peptide (residues 1-19 of SEQ ID NO:1). In specific aspects, the soluble p97 polypeptide comprises or consists of residues 20-709, 20-710, 20-711 or 20-712 of SEQ ID NO:1, including variants and fragments thereof.

In certain embodiments, for instance, those that employ liposomes, the p97 polypeptide sequence is a lipid soluble form of a p97 polypeptide. For instance, certain of these and related embodiments include a p97 polypeptide that comprises all or a portion of the hydrophobic domain, optionally with or without the signal peptide.

Specific examples of p97 polypeptides and fragments are provided in Table B1 below.

TABLE B1

| | SEQ |
|---|---|
| | ID |
| Exemplary p97-Based BBB-transport Peptides | NO: |
| MRGPSGALWLLLALRTVLGGMEVRWCATSDPEQHKCGNMS | 1 |
| EAFREAGIQPSLLCVRGTSADHCVQLIAAQEADAITLDGG | |
| AIYEAGKEHGLKPVVGEVYDQEVGTSYYAVAVVRRSSHVT | |
| IDTLKGVKSCHTGINRTVGWNVPVGYLVESGRLSVMGCDV | |
| LKAVSDYFGGSCVPGAGETSYSESLCRLCRGDSSGEGVCD | |
| KSPLERYYDYSGAFRCLAEGAGDVAFVKHSTVLENTDGKT | |
| LPSWGQALLSQDFELLCRDGSRADVTEWRQCHLARVPAHA | |
| VVVRADTDGGLIFRLLNEGQRLFSHEGSSFQMFSSEAYGQ | |
| KDLLFKDSTSELVPIATQTYEAWLGHEYLHAMKGLLCDPN | |
| RLPPYLRWCVLSTPEIQKCGDMAVAFRRQRLKPEIQCVSA | |
| KSPQHCMERIQAEQVDAVTLSGEDIYTAGKTYGLVPAAGE | |
| HYAPEDSSNSYYVVAVVRRDSSHAFTLDELRGKRSCHAGF | |
| GSPAGWDVPVGALIQRGFIRPKDCDVLTAVSEFFNASCVP | |
| VNNPKNYPSSLCALCVGDEQGRNKCVGNSQERYYGYRGAF | |
| RCLVENAGDVAFVRHTTVFDNTNGHNSEPWAAELRSEDYE | |
| LLCPNGARAEVSQFAACNLAQIPPHAVMVRPDTNIFTVYG | |
| LLDKAQDLFGDDHNKNGFKMFDSSNYHGQDLLFKDATVRA | |
| VPVGEKTTYRGWLGLDYVAALEGMSSQQCSGAAAPAPGAP | |
| LLPLLLPALAARLLPPAL (full-length human p97) | |
| GMEVRWCATSDPEQHKCGNMSEAFREAGIQPSLLCVRGTS | 2 |
| ADHCVQLIAAQEADAITLDGGAIYEAGKEHGLKPVVGEVY | |
| DQEVGTSYYAVAVVRRSSHVTIDTLKGVKSCHTGINRTVG | |
| WNVPVGYLVESGRLSVMGCDVLKAVSDYFGGSCVPGAGET | |
| SYSESLCRLCRGDSSGEGVCDKSPLERYYDYSGAFRCLAE | |
| GAGDVAFVKHSTVLENTDGKTLPSWGQALLSQDFELLCRD | |
| GSRADVTEWRQCHLARVPAHAVVVRADTDGGLIFRLLNEG | |
| QRLFSHEGSSFQMFSSEAYGQKDLLFKDSTSELVPIATQT | |
| YEAWLGHEYLHAMKGLLCDPNRLPPYLRWCVLSTPEIQKC | |
| GDMAVAFRRQRLKPEIQCVSAKSPQHCMERIQAEQVDAVT | |
| LSGEDIYTAGKTYGLVPAAGEHYAPEDSSNSYYVVAVVRR | |
| DSSHAFTLDELRGKRSCHAGFGSPAGWDVPVGALIQRGFI | |
| RPKDCDVLTAVSEFFNASCVPVNNPKNYPSSLCALCVGDE | |
| QGRNKCVGNSQERYYGYRGAFRCLVENAGDVAFVRHTTVF | |
| DNTNGHNSEPWAAELRSEDYELLCPNGARAEVSQFAACNL | |
| AQIPPHAVMVRPDTNIFTVYGLLDKAQDLFGDDHNKNGFK | |
| MFDSSNYHGQDLLFKDATVRAVPVGEKTTYRGWLGLDYVA | |
| ALEGMSSQQCSG (residues 20-711 of human p97) | |
| WCATSDPEQHK | 3 |
| RSSHVTIDTLK | 4 |
| SSHVTIDTLKGVK | 5 |
| LCRGDSSGEGVCDK | 6 |
| GDSSGEGVCDKSPLER | 7 |
| YYDYSGAFR | 8 |
| ADVTEWR | 9 |
| VPAHAVVVR | 10 |
| ADTDGGLIFR | 11 |
| CGDMAVAFR | 12 |
| LKPEIQCVSAK | 13 |
| DSSHAFTLDELR | 14 |
| SEDYELLCPNGAR | 15 |
| AQDLFGDDHNKNGFK | 16 |
| FSSEAYGQKDLLFKDSTSELVPIATQTYEAWLGHEYLHAM | 17 |
| ERIQAEQVDAVTLSGEDIYTAGKTYGLVPAAGEHYAPEDS | 18 |
| SNSYYVVAVVRRDSSHAFTLDELRGKRSCHAGFGSPAGWD | |
| VPVGALIQRGFIRPKDCDVLTAVSEFFNASCVPVNNPKNY | |
| PSSLCALCVGDEQGRNKCVGNSQERYYGYRGAFRCLVENA | |
| GDVAFVRHTTVFDNTNGHNSEPWAAELRSEDYELLCPNGA | |
| RAEVSQFAACNLAQIPPHAVM | |
| VRPDTNIFTVYGLLDKAQDLFGDDHNKNGFKM | 19 |

In certain other embodiments, the p97 fragment or variant is capable of specifically binding to a p97 receptor, an LRP1 receptor and/or an LRP1B receptor.

Additional variants and fragments of reference p97 polypeptides and other reference polypeptides are described in greater detail below.

Receptor Associated Protein (RAP).

In some embodiments, the BBB-transport moiety comprises or consists of a RAP sequence, or an active variant or fragment or analog thereof. The amino acid sequence of mature RAP (w/o signal sequence) is shown in Table B2 below.

TABLE B2

| | SEQ ID |
|---|---|
| Mature RAP Amino Acid Sequence | NO: |
| YSREKNQPKPSPKRESGEEFRMEKLNQLWEKAQRLHLPPV | 20 |
| RLAELHADLKIQERDELAWKKLKLDGLDEDGEKEARLIRN | |
| LNVILAKYGLDGKKDARQVTSNSLSGTQEDGLDDPRLEKL | |
| WHKAKTSGKFSGEELDKLWREFLHHKEKVHEYNVLLETLS | |
| RTEEIHENVISPSDLSDIKGSVLHSRHTELKEKLRSINQG | |
| LDRLRRVSHQGYSTEAEFEEPRVIDLWDLAQSANLTDKEL | |
| EAFREELKHFEAKIEKHNHYQKQLEIAHEKLRHAESVGDG | |
| ERVSRSREKHALLEGRTKELGYTVKKHLQDLSGRISRARH | |
| NEL | |

In certain embodiments, a RAP fragment is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, or 320 or more amino acids in length, including all integers and ranges in between, and which may comprise all or a portion of the RAP sequence set forth in SEQ ID NO:20.

Further examples of RAPs, including RAP variants, are described, for instance, in U.S. Application Nos. 2009/0269346; 2009/0281024; 2010/0028370; 2010/0183581; 2010/0210517; 2011/0142763; and U.S. Pat. Nos. 7,560,431; 7,569,544; 7,700,554; 7,829,537; 7,977,317; and 8,440,629, each of which is incorporated by reference in its entirety. Additional variants and fragments of reference RAP sequences and other reference polypeptides are described in greater detail below.

Aprotinin and Related Sequences.

In some embodiments, the BBB-transport moiety comprises or consists of an aprotinin polypeptide or peptide, an aprotinin-related polypeptide or peptide, or an active variant or fragment or analog thereof. Particular examples include aprotinin fragments of about 15-58 contiguous amino acids of the aprotinin sequence set forth in SEQ ID NO:21; or fragments of about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 37, 38, 39, 40, 41, 42, 43, 44, 45, 46, 47, 48, 49, 50, 51, 52, 53, 54, 55, 56, 57, 58 contiguous amino acids of SEQ ID NO:21.

The sequences of exemplary aprotinin peptide/analogs are provided in Table B3 below.

TABLE B3

| | SEQ ID |
|---|---|
| Exemplary BBB-transport Peptides | NO: |
| RPDFCLEPPYTGPCKARIIRYFYNAKAGLCQTFVYGGCRA | 21 |
| KRNNFKSAEDCMRTCGGA (Aprotinin) | |
| TFVYGGCRAKRNNFKSAED (Aprotinin fragment) | 22 |
| TFFYGGCRGKRNNFKTEEY (Angiopep-1) | 23 |
| TFFYGGSRGKRNNFKTEEY (Angiopep-2) | 24 |
| TFVYGGCRAKRNNFKSAED | 25 |
| TFQYGGCMGNGNNFVTEKE | 26 |
| PFFYGGCGGNRNNFDTEEY | 27 |
| SFYYGGCLGNKNNYLREEE | 28 |
| TFFYGGCRAKRNNFKRAKY | 29 |
| TFFYGGCRGKRNNFKRAKY | 30 |
| TFFYGGCRAKKNNYKRAKY | 31 |
| TFFYGGCRGKKNNFKRAKY | 32 |
| TFQYGGCRAKRNNFKRAKY | 33 |
| TFQYGGCRGKKNNFKRAKY | 34 |
| TFFYGGCLGKRNNFKRAKY | 35 |
| TFFYGGSLGKRNNFKRAKY | 36 |
| PFFYGGCGGKKNNFKRAKY | 37 |
| TFFYGGCRGKGNNYKRAKY | 38 |
| PFFYGGCRGKRNNFLRAKY | 39 |
| TFFYGGCRGKRNNFKREKY | 40 |
| PFFYGGCRAKKNNFKRAKE | 41 |
| TFFYGGCRGKRNNFKRAKD | 42 |
| TFFYGGCRAKRNNFDRAKY | 43 |
| TFFYGGCRGKKNNFKRAEY | 44 |
| PFFYGGCGANRNNFKRAKY | 45 |
| TFFYGGCGGKKNNFKTAKY | 46 |
| TFFYGGCRGNRNNFLRAKY | 47 |
| TFFYGGCRGNRNNFKTAKY | 48 |
| TFFYGGSRGNRNNFKTAKY | 49 |
| TFFYGGCLGNGNNFKRAKY | 50 |
| TFFYGGCLGNRNNFLRAKY | 51 |
| TFFYGGCLGNRNNFKTAKY | 52 |
| TFFYGGCRGNGNNFKSAKY | 53 |
| TFFYGGCRGKKNNFDREKY | 54 |
| TFFYGGCRGKRNNFLREKE | 55 |
| TFFYGGCRGKGNNFDRAKY | 56 |
| TFFYGGSRGKGNNFDRAKY | 57 |
| TFFYGGCRGNGNNFVTAKY | 58 |
| PFFYGGCGGKGNNYVTAKY | 59 |
| TFFYGGCLGKGNNFLTAKY | 60 |
| SFFYGGCLGNKNNFLTAKY | 61 |
| TFFYGGCGGNKNNFVREKY | 62 |
| LGNKNNYVREKY | 63 |
| TFFYGGCGGNGNNFLTAKY | 64 |
| TFFYGGCRGNRNNFLTAEY | 65 |
| TFFYGGCRGNGNNFKSAEY | 66 |
| PFFYGGCLGNKNNFKTAEY | 67 |
| TFFYGGCRGNRNNFKTEEY | 68 |
| TFFYGGCRGKRNNFKTEED | 69 |
| PFFYGGCGGNGNNFVREKY | 70 |
| SFFYGGCMGNGNNFVREKY | 71 |
| PFFYGGCGGNGNNFLREKY | 72 |
| TFFYGGCLGNGNNFVREKY | 73 |
| SFFYGGCLGNGNNYLREKY | 74 |
| TFFYGGSLGNGNNFVREKY | 75 |
| TFFYGGCRGNGNNFVTAEY | 76 |
| TFFYGGCLGKGNNFVSAEY | 77 |
| TFFYGGCLGNRNNFDRAEY | 78 |
| TFFYGGCLGNRNNFLREEY | 79 |
| TFFYGGCLGNKNNYLREEY | 80 |
| PFFYGGCGGNRNNYLREEY | 81 |
| PFFYGGSGGNRNNYLREEY | 82 |
| MRPDFCLEPPYTGPCVARI | 83 |
| ARIIRYFYNAKAGLCQTFVYG | 84 |
| YGGCRAKRNNYKSAEDCMRTCG | 85 |
| PDFCLEPPYTGPCVARIIRYFY | 86 |
| TFFYGGCRGKRNNFKTEEY | 87 |
| KFFYGGCRGKRNNFKTEEY | 88 |
| TFYYGGCRGKRNNYKTEEY | 89 |
| TFFYGGSRGKRNNFKTEEY | 90 |
| CTFFYGCCRGKRNNFKTEEY | 91 |
| TFFYGGCRGKRNNFKTEEYC | 92 |
| TFFYGGKRGKRNNFKTEEY | 93 |
| TFFYGGCRGKRNNFKTKRY | 94 |
| TFFYGGKRGKRNNFKTAEY | 95 |
| TFFYGGKRGKRNNFKRAGY | 96 |
| TFFYGGKRGKRNNFKREKY | 97 |
| TFFYGGKRGKRNNFKTAKY | 98 |
| TFFYGGCLGNRNNFKTEEY | 99 |
| TFFYGCGRGKRNNFKTEEY | 100 |
| TFFYGGRCGKRNNFKTEEY | 101 |
| TFFYGGCLGNGNNFDTEEEQ | 102 |
| TFQYGGCRGKRNNFKTEEY | 103 |
| YNKEFGTFNTKGCERGYRF | 104 |
| RFKYGGCLGNMNNFETLEE | 105 |
| RFKYGGCLGNKNNFLRLKY | 106 |
| RFKYGGCLGNKNNYLRLKY | 107 |
| KTKRKRKKQRVKIAYEEIFKNY | 108 |
| KTKRKRKKQRVKIAY | 109 |
| RGGRLSYSRRFSTSTGR | 110 |
| RRLSYSRRRF | 111 |
| RQIKIWFQNRRMKWKK | 112 |
| TFFYGGSRGKRNNFKTEEY | 113 |
| MRPDFCLEPPYTGPCVARIIRYFYNAKAGLCQTFVYGGCR | 114 |
| AKRNNFKSAEDCMRTCGGA | |
| TFFYGGCRGKRNNFKTKEY | 115 |
| TFFYGGCRAKRNNFKRAKY | 116 |
| NAKAGLCQTFVYGGCLAKRNNFESAEDCMRTCGGA | 117 |
| YGGCRAKRNNFKSAEDCMRTCGGA | 118 |
| GLCQTFVYGGCRAKRNNFKSAE | 119 |
| LCQTFVYGGCEAKRNNFKSA | 120 |
| TFFYGGSRGKRNNFKTEEY | 121 |
| RFFYGGSRGKRNNFKTEEY | 122 |
| RFFYGGSRGKRNNFKTEEY | 123 |
| RFFYGGSRGKRNNFRTEEY | 124 |
| TFFYGGSRGKRNNFRTEEY | 125 |
| TFFYGGSRGRRNNFRTEEY | 126 |
| CTFFYGGSRGKRNNFKTEEY | 127 |
| TFFYGGSRGKRNNFKTEEYC | 128 |
| CTFFYGGSRGRRNNFRTEEY | 129 |
| TFFYGGSRGRRNNFRTEEYC | 130 |

Certain embodiments employ aprotinin-peptide coated liposomes or nanoparticles. Additional examples of aprotinin-based BBB targeting peptides are described, for example, in U.S. Application Nos. 2006/0189515; 2008/0299039; 2010/0297120; 2011/0171128; and 2012/0277158; U.S. Pat. Nos. 7,902,156 and 7,557,182; and PCT Publication Nos. WO 2006/086870 and WO 2004/060403, each of which is incorporated by reference in its entirety. Additional variants and fragments of reference aprotinin and aprotinin-related polypeptides and other reference polypeptides are described in greater detail below.

Protein Transduction Domains (PTD5).

In certain embodiments the BBB-transport moiety comprises or consists of an absorptive mediated protein transduction domain (PTD). PTD5 are typically amino acid sequences located on transcription factors which allow transport of larger molecules across cell membranes. Particular examples of PTD5 include TAT (e.g., HIV-1 TAT), the homeodomain of Antennapedia, SynB peptides (e.g., SynB1, SynB3, SynB5), penetratin, polyarginine, and others. Residues 37-48 (core domain) of TAT bind to LRP domains II, II, and IV and residues 49-57 promote absorptive endocytosis across cell membranes. The amino acid sequences of TAT (LAI/Bru strain), penetratin, and SynB (PG-1) peptides are shown in Table B4 below.

TABLE B4

| | SEQ ID |
|---|---|
| Exemplary PTD Sequences | NO: |
| MEPVDPRLEPWKHPGSQPKTACTTCYCKKCCFHCQVCFTT | 131 |
| KALGISYGRKKRRQRRRPPQGSQTHQVSLSKQPTSQPRGD | |
| PTGPKE (full-length HIV-1 TAT; | |
| isolate BRU/LAI) | |
| FTTKALGISYGRKKRRQRRR (residues 37-57 of TAT) | 132 |
| RQIKIWFQNRRMKWKK (Penetratin) | 133 |
| RGGRLCYCRRRFCVCVGR (PG-1) | 134 |
| RGGRLSYSRRRFSTSTGRA (SynB1) | 135 |
| RRLSYSRRRF (SynB3) | 136 |
| RGGRLAYLRRRWAVLGR (SynB5) | 137 |

Additional variants and fragments of reference PTD sequences and other reference polypeptides are described in greater detail below.

hLDLR-Binding Peptides.

In certain embodiments, the BBB-transport moiety is a peptide that binds to human low-density lipoprotein receptor (hLDLR). LDLR is a transmembrane protein of 839 amino acids comprising three regions: the extracellular region (1-768), the transmembrane region (768-790) and the cytoplasmic region (790-839). The extracellular region is divided into two subregions: that of LDL binding (1-322) and that outside the LDL binding region (322-768) (see WO2007/014992). Certain hLDLR-binding peptides are about, at least about, or up to about 5, 6, 7, 8, 9, 10, 11, 12, 13, 14, 15, 16, 17, 18, 19, 20, 21, 22, 23, 24, 25, 26, 27, 28, 29, 30 amino acids in length. Such peptides may contain the sequence of one or more exemplary hLDLR-binding peptides shown in Table B5 below.

TABLE B5

| | SEQ ID |
|---|---|
| Exemplary hLDLR-Binding Peptides | NO: |
| HLDCMPRGCFRN | 138 |
| ACQVKSMPRC | 139 |
| ACTTPMPRLC | 140 |
| ACKAPQMPRC | 141 |
| ACLNPSMPRC | 142 |
| ACLVSSMPRC | 143 |
| ACLQPMPRLC | 144 |
| ACPVSSMPRC | 145 |
| ACQSPMPRLC | 146 |
| ACLTPMPRLC | 147 |
| DSGLCMPRLRGCDPR | 148 |
| ACMPRLRGCA | 149 |
| ASGLCMPRLRGCDPR | 150 |
| DAGLCMPRLRGCDPR | 151 |
| DSALCMPRLRGCDPR | 152 |
| DSGACMPRLRGCDPR | 153 |
| DSGLCMPRARGCDPR | 154 |
| DSGLCMPRLAGCDPR | 155 |
| DSGLCMPRLRACDPR | 156 |
| DSGLCMPRLRGCAPR | 157 |
| DSGLCMPRLRGCDAR | 158 |
| DSGLCMPRLRGCDPA | 159 |
| SGLCMPRLRGCDPR | 160 |
| GLCMPRLRGCDPR | 161 |
| CMPRLRGC | 162 |
| CMPRARGC | 163 |
| CMPRLAGC | 164 |
| CMPRLRAC | 165 |
| CMPRLKGC | 166 |
| (D)-CMPRLRGC | 167 |
| CMPR-(D)-LRGC | 168 |
| CMPRL-(D)-RGC | 169 |
| CMPRLRG-(D)-C | 170 |
| (D)-CMPRLRG-(D)- | 171 |
| (D)-CMPRLRSarC | 172 |
| (D)-CMPRKRGC | 173 |
| (D)-CMPRLRC | 174 |
| (D)-CMPRLCR | 175 |
| (D)-CMPRCLRG | 176 |
| CMPRGC | 177 |
| PenMPRLRGC | 178 |
| (D)-PenMPRLRGC | 179 |
| (D)-CMPRLRGPen | 180 |
| PenMPRLRGPen | 181 |
| (D)-PenMPRLRGPen | 182 |
| (D)-PenMPRLRG-(D)-Pen | 183 |
| MpaMPRLRGC | 184 |
| MTVMPTGLWNPLIPS | 185 |
| SASWFAVPIPPLRLE | 186 |
| MTPMSTPRMLPVYVA | 187 |
| MTATHLSTLFQPLTY | 188 |
| MSPIPPAASTWANTL | 189 |
| MTANPLQNAPGPLSL | 190 |
| MQTAPPPPLTRVQWS | 191 |
| GTPRMHIPLNVDHLP | 192 |
| LTLPPISGLSSYPLP | 193 |
| TPSAHAMALQSLSVG | 194 |
| LTLPPISGLSSYPLP | 195 |
| MGTLNAPTAYPQDSL | 196 |
| LTNPPAYLPQNTDPH | 197 |
| MGLPLPYIQTILHTP | 198 |
| SAALIAMSSFKSITA | 199 |
| SGFAFARSVPTESRR | 200 |
| MTSPYMSLPPSTDD | 201 |
| LTNPPAYLPQNTDPH | 202 |
| A1-M-A2-R-L-AR-A3-A4 | 203 |
| wherein A1 and A4 independently represent | |
| cysteine (Cys) or an analogue or isostere | |
| thereof, A2 represents proline (Pro) | |
| or an analogue or isostere thereof, | |
| and A3 represents glycine (Gly) or an | |
| analogue or isostere thereof | |
| A1-M-A2-R-L-R-G-C (I′) wherein A1 rep- | 204 |
| resents Cys, (D)-cys, Pen, or (D)-Pen; | |
| and A2 represents Pro, Pip or Thz | |
| (D)-cys-Met-Pip-Arg-Leu-Arg-Gly-Cys | 205 |
| (D)-pen-Met-Pip-Arg-Leu-Arg-Gly-Cys | 206 |
| Pen-Met-Pip-Arg-Leu-Arg-Gly-Cys | 207 |
| (D)-cys-Met-Thz-Arg-Leu-Arg-Gly-Cys | 208 |
| (D)-pen-Met-Thz-Arg-Leu-Arg-Gly-Cys | 209 |
| Pen-Met-Thz-Arg-Leu-Arg-Gly-Cys | 210 |
| (D)-cys-Met-Thz-Arg-Leu-Arg-Sar-Cys | 211 |
| (D)-Cys-Met-Thz-Arg-Leu-Arg-Gly- Pen | 212 |
| (D)-Cys-Met-Thz-Arg-Leu-Arg-Sar-Pen | 213 |
| (D)-Cys-Met-Pip-Arg-Leu-Arg-Sar-Cys | 214 |
| (D)-Cys-Met-Pip-Arg-Leu-Arg-Gly-Pen | 215 |
| (D)-Cys-Met-Pip-Arg-Leu-Arg-Sar-Pen | 216 |
| (D)-Cys-Met-Pro-hArg-Leu-Arg-Gly-Cys | 217 |
| (D)-Cys-Met-Pro-Agb-Leu-Arg-Gly-Cys | 218 |
| (D)-Cys-Met-Pro-Agp-Leu-Arg-Gly-Cys | 219 |
| (D)-Cys-Met-Pro-Cit-Leu-Arg-Gly-Cys | 220 |
| (D)-Cys-Met-Pro-Arg-Leu-Cit-Gly-Cy | 221 |
| (D)-Cys-Met-Pro-Arg-Leu-Arg(NO2)-Gly-Cys | 222 |
| (D)-Cys-Met-Pro-Arg-Leu-Arg-Gly- Cys | 223 |
| Cys-Met-Pro-Arg-Leu-Arg-Gly-Cys | 224 |

The hLDLR-binding peptides can be linear or cyclic peptides, composed of natural and/or non-natural amino acids. Additional examples of hLDLR-binding peptides are described, for example, in U.S. Application Nos. 2011/0230416 and 2013/0108548, each of which is incorporated by reference in its entirety.

BBB-Transport Antibodies/Ligands:

In some embodiments, the BBB-transport moiety comprises or consists of an immunoglobulin molecule, such as a monoclonal antibody, or a portion thereof (e.g., antigen binding fragment, ScFv, Fab, sdAb) that binds or specifically binds to a BBB-associated receptor, i.e., a receptor that mediates transport across the BBB. In other embodiments, the BBB-transport moiety is a natural ligand (or a variant of fragment thereof) of a BBB-associated receptor.

Typically, the BBB-associated receptor is a specific transporter protein that facilitates transfer of nutrients, hormones, insulin, and other substances across the BBB, by receptor-mediated transcytosis. Examples of BBB-associated receptors include the insulin receptor, the transferrin receptor, the leptin receptor, lipoprotein receptors such as the lipoprotein receptor-related protein (LRP-1) receptor, insulin-like growth factor (IGF) receptors such as IGF1R and IGF2R, the low-density lipoprotein receptor, the diptheria toxin receptor, and TMEM 30A (Flippase).

Accordingly, certain BBB-transport moieties include an immunoglobulin molecule, such as a monoclonal antibody, or a portion thereof (e.g., antigen binding fragment, ScFv, Fab, sdAb) that binds or specifically binds to the insulin receptor, the transferrin receptor, the leptin receptor, lipoprotein receptors such as the lipoprotein receptor-related protein (LRP-1) receptor, insulin-like growth factor (IGF) receptors such as IGF1R and IGF2R, the low-density lipoprotein receptor, the diptheria toxin receptor, or TMEM 30A (Flippase).

Specific examples of BBB-transport antibodies are described, for example, in U.S. Application Nos. 2008/0152645; 2008/0170994; 2008/0171055; 2009/0053219; 2009/0156498; 2011/0110935; and 2013/0142794; and U.S. Pat. Nos. 7,741,446; 8,053,569; 8,124,095; 8,142,781; 8,486,399; and 8,497,246; and PCT Publication Nos. WO 2013/081706; WO 2011/088409; WO 2011/044542; WO 2010/108048; WO 2009/070597; WO 2009/018122; WO 2008/022349; and WO 2007/044323, each of which is incorporated by reference in its entirety.

Examples of ligands of BBB-associated receptor ligands include insulin, transferrin and transferrin fragments, lactoferrin and lactoferrin fragments, apolipoprotein A (Apo A), apolipoprotein B (Apo B), apolipoprotein E (Apo E), melanotransferrin (supra), RAP and RAP fragments (supra), diptheria toxin (including non-toxic mutants thereof such as CRM45 and CRM197), and others described herein and known in the art. Certain BBB-transport moieties thus comprise or consist of one or more of the foregoing BBB-associated receptor ligands.

Glutathione (GSH).

In certain embodiments, the BBB-transport moiety comprises or consists of glutathione (GSH). GSH is a tripeptide with a gamma peptide linkage between the amine group of cysteine (which is attached by normal peptide linkage to a glycine) and the carboxyl group of the glutamate side-chain.

In some embodiments, the GSH moiety is operatively linked (e.g., non-covalently linked) to the Fc-modified antibody or Fc-fusion polypeptide, for instance, as part a carrier system such as a liposome. The carrier may comprise nanoparticles, polymeric nanoparticles, solid liquid nanoparticles, polymeric micelles, liposomes, microemulsions, and/or liquid-based nanoparticles. The liposomes may comprise at least one of lecithin such as soy lecithin and hydrogenated lecithin such as hydrogenated soy lecithin. In some aspects, the liposomes may comprise cholesterol, water-soluble vitamin E, and/or octadecyl amine, for instance, to increase serum resistance or charge amounts. In particular embodiments, the molar composition ratio of the liposome may be 0.5-100% of lecithin or hydrogenated lecithin, 0.005-75% of cholesterol or water-soluble vitamin E, 0.001-25% of octadecyl amine.

Specific examples include a carrier which comprises an Fc-modified antibody or Fc-fusion polypeptide (as described herein) and glutathione, where the glutathione is covalently bound to polyethylene glycol, optionally where the polyethylene glycol is covalently bound to vitamin E or a phospholipid, and the vitamin E or phospholipid is intercalated into the carrier such that the glutathione is on an outside surface of the carrier (e.g., GSH-PEG-coated liposomes). Exemplary GSH-based conjugates and carrier systems are described, for example, in U.S. Application Nos. 2007/0141133; 2008/0095836; and 2009/0123531; and U.S. Pat. Nos. 7,446,096; 7,700,564; 7,704,956; and 8,067,380, each of which is incorporated by reference in its entirety.

Fc Modifications

The CNS-targeted conjugates described herein have one or more modifications to the Fc region of the conjugated antibody or therapeutic Fc-fusion polypeptide. Such Fc modifications can alter (e.g., reduce) the binding to or interaction between the Fc region and one or more Fc ligands/receptors. Thus, variant or otherwise modified Fc regions will typically have altered Fc receptor/ligand binding characteristics and/or biological activities (e.g., Fc effector activities) relative to wild-type or commercially-employed Fc region(s).

Examples of modified Fc regions include those having mutated sequences, for instance, by substitution, insertion, deletion, or truncation of one or more amino acids relative to a wild-type or commercially-employed sequence, hybrid Fc polypeptides composed of domains from different immunoglobulin classes/subclasses, Fc polypeptides having altered glycosylation/sialylation patterns, and Fc polypeptides that are modified or derivatized, for example, by biotinylation (see, e.g., US Application No. 2010/0209424), phosphorylation, sulfation, etc., or any combination of the foregoing.

In certain instances, Fc modifications can alter (e.g., increase, decrease) the binding properties of the Fc region to one or more particular FcRs (e.g., FcγRI, FcγRIIa, FcγRIIb, FcγRIIc, FcγRIIIa, FcγRIIIb), its pharmacokinetic properties (e.g., stability or half-life, bioavailability, tissue distribution, volume of distribution, concentration, elimination rate constant, elimination rate, area under the curve (AUC), clearance, Cₘₐₓ, tₘₐₓ, Cₘₐₓ, fluctuation), its immunogenicity, its complement fixation or activation, and/or the CDC, ADCC, and/or ADCP-related activities of the Fc region, among other properties described herein, relative to a corresponding wild-type or commercial Fc sequence.

The “Fc region” of is usually derived from the heavy chain of an immunoglobulin (Ig) molecule. A typical Ig molecule is composed of two heavy chains and two light chains. The heavy chains can be divided into at least three functional regions: the Fd region, the Fc region (fragment crystallizable region), and the hinge region, the latter being found only in IgG, IgA, and IgD immunoglobulins. The Fd region comprises the variable (V_{H}) and constant (CH₁) domains of the heavy chains, and together with the variable (V_{L}) and constant (C_{L}) domains of the light chains forms the antigen-binding fragment or Fab region. In certain embodiments, however, the hinge region is included within the meaning of the Fc region, for example, for deletions or other Fc modifications.

The Fc region of IgG, IgA, and IgD immunoglobulins comprises the heavy chain constant domains 2 and 3, designated respectively as CH₂ and CH₃ regions; and the Fc region of IgE and IgM immunoglobulins comprises the heavy chain constant domains 2, 3, and 4, designated respectively as CH₂, CH₃, and CH₄ regions. The Fc region is mainly responsible for the immunoglobulin effector functions, which include, for example, complement fixation and binding to cognate Fc receptors/ligands of effector cells.

The hinge region (found in IgG, IgA, and IgD) acts as a flexible spacer that allows the Fab portion to move freely in space relative to the Fc region. In contrast to the constant regions, the hinge regions are structurally diverse, varying in both sequence and length among immunoglobulin classes and subclasses. The hinge region may also contain one or more glycosylation site(s), which include a number of structurally distinct types of sites for carbohydrate attachment. For example, IgA1 contains five glycosylation sites within a 17 amino acid segment of the hinge region, conferring significant resistance of the hinge region polypeptide to intestinal proteases. Residues in the hinge proximal region of the CH₂ domain can also influence the specificity of the interaction between an immunoglobulin and its respective Fc receptor(s) (see, e.g., Shin et al., Intern. Rev. Immunol. 10:177-186, 1993). Hence, certain embodiments include modifications to the hinge region of an antibody or Fc-fusion polypeptide described herein.

The term “Fc region” or “Fc fragment” or “Fc” as used herein, refers to a protein that contains one or more of a CH₂ region, a CH₃ region, and/or a CH₄ region from one or more selected immunoglobulin(s), including fragments and variants and combinations thereof. An “Fc region” may also include one or more hinge region(s) of the heavy chain constant region of an immunoglobulin. In certain embodiments, the Fc region does not refer to one or more of the CH₁, C_{L} V_{L}, and/or V_{H} regions of an immunoglobulin.

The Fc region can be modified at any one or more of the CH₂ region, CH₃ region, CH₄ region, and/or hinge region(s) of any one or more immunoglobulin classes, including but not limited to IgA, IgD, IgE, IgG, IgM, including subclasses and combinations thereof. In some embodiments, the modified Fc region is derived from an IgA immunoglobulin, including subclasses IgA1 and/or IgA2. In certain embodiments, the modified Fc region is derived from an IgD immunoglobulin. In particular embodiments, the modified Fc region is derived from an IgE immunoglobulin. In some embodiments, the modified Fc region is derived from an IgG immunoglobulin, including subclasses IgG1, IgG2, IgG2, IgG3, and/or IgG4. In certain embodiments, the modified Fc region is derived from an IgM immunoglobulin. Certain embodiments include a modified Fc region that has a full or partial deletion of any one or more of the CH₂ region, the CH₃ region, the CH₄ region, and/or the hinge region(s) of any one or more immunoglobulin classes.

Fc regions demonstrate specific binding for one or more Fc-receptors (FcRs). Examples of classes of Fc receptors include Fey receptors (FcγR), Fcα receptors (FcαR), Fcε receptors (FcεR), and the neonatal Fc receptor (FcRn). Thus, certain modified Fc regions have altered (e.g., decreased) binding to (or affinity for) one or more FcγRs, relative to that of an unmodified Fc region. In some embodiments, modified Fc regions have altered (e.g., decreased) binding to FcαRs, relative to that of an unmodified Fc region. In other embodiments, modified Fc regions have altered (e.g., decreased) binding to FcεRs (e.g., FcαRI), relative to that of an unmodified Fc region. In particular embodiments, modified Fc regions have altered (e.g., decreased) binding to FcRn, relative to that of an unmodified Fc region. In certain embodiments, the binding (or affinity) of an Fc region to one or more selected FcR(s) is altered (e.g., decreased) relative to that of an unmodified Fc region, typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between).

Examples of FcγRs include FcγRI, FcγRIIa, FcγRIIb, FcγRIIc, FcγRIIIa, and FcγRIIIb. FcγRI (CD64) is expressed on macrophages and dendritic cells and plays a role in phagocytosis, respiratory burst, cytokine stimulation, and dendritic cell endocytic transport. Expression of FcγRI is upregulated by both GM-CSF and γ-interferon (γ-IFN) and downregulated by interleukin-4 (IL-4). FcγRIIa is expressed on polymorphonuclear leukocytes (PMN), macrophages, dendritic cells, and mast cells. FcγRIIa plays a role in phagocytosis, respiratory burst, and cytokine stimulation. Expression of FcγRIIa is upregulated by GM-CSF and γ-IFN, and decreased by IL-4. FcγIIb is expressed on B cells, PMN, macrophages, and mast cells. FcγIIb inhibits immunoreceptor tyrosine-based activation motif (ITAM) mediated responses, and is thus an inhibitory receptor. Expression of FcγRIIc is upregulated by intravenous immunoglobulin (IVIG) and IL-4 and decreased by γ-IFN. FcγRIIc is expressed on NK cells. FcγRIIIa is expressed on natural killer (NK) cells, macrophages, mast cells, and platelets. This receptor participates in phagocytosis, respiratory burst, cytokine stimulation, platelet aggregation and degranulation, and NK-mediated ADCC. Expression of FcγRIII is upregulated by C5a, TGF-β, and γ-IFN and downregulated by IL-4. Fc γ RIIIb is a GPI-linked receptor expressed on PMN.

Certain modified Fc regions have altered (e.g., decreased) binding to FcγRI, relative to that of an unmodified Fc region. Some embodiments have altered (e.g., decreased) binding to FcγRIIa, relative to that of an unmodified Fc region. Particular modified Fc regions have altered (e.g., decreased) binding to FcγRIIb, relative to that of an unmodified Fc region. Certain modified Fc regions have altered (e.g., decreased) binding to FcγRIIc, relative to that of an unmodified Fc region. Some modified Fc regions have altered (e.g., decreased) binding to FcγRIIIa, relative to that of an unmodified Fc region. Specific modified Fc regions have altered (e.g., decreased) binding to FcγRIIIb, relative to that of an unmodified Fc region.

FcαRs include FcαRI (CD89). FcαRI is found on the surface of neutrophils, eosinophils, monocytes, certain macrophages (e.g., Kupffer cells), and certain dendritic cells. FcαRI is composed of two extracellular Ig-like domains, is a member of both the immunoglobulin superfamily and the multi-chain immune recognition receptor (MIRR) family, and signals by associating with two FcRγ signaling chains.

FcεRs include FcεRI and FcεRII. The high-affinity receptor FcεRI is a member of the immunoglobulin superfamily, is expressed on epidermal Langerhans cells, eosinophils, mast cells and basophils, and plays a major role in controlling allergic responses. FcεRI is also expressed on antigen-presenting cells, and regulates the production pro-inflammatory cytokines. The low-affinity receptor FcεRII (CD23) is a C-type lectin that can function as a membrane-bound or soluble receptor. FcεRII regulates B cell growth and differentiation, and blocks IgE-binding of eosinophils, monocytes, and basophils. Certain modified Fc regions have altered (e.g., decreased) binding to FcεRI, relative to that of an unmodified Fc region. Some modified Fc regions have altered (e.g., decreased) binding to FcεRII, relative to that of an unmodified Fc region.

Certain modified Fc regions have altered (e.g., decreased) binding to FcRn, relative to that of an unmodified Fc region. Examples include a modified Fc region which comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386, 388, 400, 413, 415, 424, 433, 434, 435, 436, 439, and 447 of the Fc region, where the numbering of the residues in the Fc region is that of the EU index, as described in PCT Publication No. WO 2000/42072.

Additional examples of substitutions for altering binding to FcRn can be made at position 250, 314, or 428 alone, or in any combinations thereof, such as at positions 250 and 428, or at positions 250 and 314, or at positions 314 and 428, or at positions 250, 314, and 428, with positions 250 and 428 as a preferred combination. For each position, the substituting amino acid may be any amino acid residue different from that present in that position of the unmodified antibody. For position 250, the substituting amino acid residue can be any amino acid residue other than threonine, including, but not limited to, alanine, cysteine, aspartic acid, glutamic acid, phenylalanine, glycine, histidine, isoleucine, lysine, leucine, methionine, asparagine, proline, glutamine, arginine, serine, valine, tryptophan, or tyrosine. For position 314, the substituting amino acid residue can be any amino acid residue other than leucine, including, but not limited to, alanine, cysteine, aspartic acid, glutamic acid, phenylalanine, glycine, histidine, isoleucine, lysine, methionine, asparagine, proline, glutamine, arginine, serine, threonine, valine, tryptophan, or tyrosine. For position 428, the substituting amino acid residues can be any amino acid residue other than methionine, including, but not limited to, alanine, cysteine, aspartic acid, glutamic acid, phenylalanine, glycine, histidine, isoleucine, lysine, leucine, asparagine, proline, glutamine, arginine, serine, threonine, valine, tryptophan, or tyrosine. Here, numbering of the residues in the heavy chain is that of the EU index (Kabat et al.).

Additional non-limiting examples of Fc modifications that alter its binding to the FcRn are described, for example, in U.S. Pat. Nos. 7,217,797 and 7,732,570; and U.S. Application Nos. US 2010/0143254 and 2010/0143254

Certain modifications to the Fc region of an antibody or Fc-fusion polypeptide alter (e.g., reduce) one or more of the biological effects of its interactions with an Fc ligand. Table F1 summarizes the characteristics of certain FcRs.

TABLE F1
Exemplary Fc-Receptors

| | Primary | | | |
|---|---|---|---|---|
| | Antibody | | | Exemplary Effects Following Binding |
| Receptor | Ligand | Ligand Affinity | Cell Distribution | to Fc Ligand |
| FcγRI | IgG1 and | High (Kd ~10⁻⁹M) | Macrophages | Phagocytosis |
| (CD64) | IgG3 | | Neutrophils | Cell activation |
| | | | Eosinophils | Activation of respiratory burst |
| | | | Dendritic cells | Induction of microbe killing |
| FcγRIIa | IgG | Low (Kd > 10⁻⁷M) | Macrophages | Phagocytosis |
| (CD32) | | | Neutrophils | Degranulation (eosinophils) |
| | | | Eosinophils | |
| | | | Platelets | |
| | | | Langerhans cells | |
| FcγRIIb1 | IgG | Low (Kd > 10⁻⁷M) | B Cells | No phagocytosis |
| (CD32) | | | Mast cells | Inhibition of cell activity |
| FcγRIIb2 | IgG | Low (Kd > 10⁻⁷M) | Macrophages | Phagocytosis |
| (CD32) | | | Neutrophils | Inhibition of cell activity |
| | | | Eosinophils | |
| FcγRIIIa | IgG | Low (Kd > 10⁻⁶M) | NK cells | Induction of antibody-dependent cell- |
| (CD16a) | | | Macrophages | mediated cytotoxicity (ADCC) |
| | | | (certain tissues) | Induction of cytokine release by |
| | | | | macrophages |
| FcγRIIIb | IgG | Low (Kd > 10⁻⁶M) | Eosinophils | Induction of microbe killing |
| (CD16b) | | | Macrophages | |
| | | | Neutrophils | |
| | | | Mast cells | |
| | | | Follicular dendritic | |
| | | | cells | |
| FcεRI | IgE | High (Kd ~10⁻¹⁰M) | Mast cells | Degranulation |
| | | | Eosinophils | |
| | | | Basophils | |
| | | | Langerhans cells | |
| FcεRII | IgE | Low (Kd > 10⁻⁷M) | B cells | Possible adhesion molecule |
| (CD23) | | | Eosinophils | |
| | | | Langerhans cells | |
| FcαRI | IgA | Low (Kd > 10⁻⁶M) | Monocytes | Phagocytosis |
| (CD89) | | | Macrophages | Induction of microbe killing |
| | | | Neutrophils | |
| | | | Eosinophils | |
| Fcα/μR | IgA and | High for IgM, | B cells | Endocytosis |
| | IgM | Moderate for IgA | Mesangial cells | Induction of microbe killing |
| | | | Macrophages | |
| FcRn | IgG | | Monocytes | Transfers IgG from a mother to fetus |
| | | | Macrophages | through the placenta |
| | | | Dendrite cells | Transfers IgG from a mother to infant |
| | | | Epithelial cells | in milk |
| | | | Endothelial cells | Protects IgG from degradation |
| | | | Hepatocytes | |

Certain modified Fc regions may have altered effector functions, relative to a corresponding, wild-type Fc sequence. For example, such Fc regions may have altered (e.g., decreased) complement fixation or activation, C1q binding affinity, complement-dependent cytotoxicity (CDC)-related activity, antibody-dependent cellular cytotoxicity (ADCC)-related activity, and/or antibody-dependent cell phagocytosis (ADCP)-related activity, relative to a corresponding, wild-type or commercial Fc sequence. As merely one illustrative example, an Fc region may comprise a deletion or substitution in a complement-binding site, such as a C1q-binding site, and/or a deletion or substitution in an ADCC site. Examples of such deletions/substitutions are described, for example, in U.S. Pat. No. 7,030,226. Particular examples include human IgG1 Fc regions having the following series of mutations (E233P/L234V/L235A/G236+A327G/A330S/P331S), which have reduced binding to FcγRI and reduced ADCC and CDC activities (see, e.g., Armour et al., Eur. J. Immunol. 29:2613-24, 1999). Also included are human IgG1 Fc regions having a K322A mutation, which have reduced binding to C1q and reduced CDC-related activity (see, e.g., Idusogie et al., J. Immunol. 164:4178-84, 2000). Many Fc effector functions, such as ADCC, can be assayed according to routine techniques in the art. (see, e.g., Zuckerman et al., CRC Crit Rev Microbiol. 7:1-26, 1978). Useful effector cells for such assays includes, but are not limited to, natural killer (NK) cells, macrophages, and other peripheral blood mononuclear cells (PBMC). Alternatively, or additionally, certain Fc effector functions may be assessed in vivo, for example, by employing an animal model described in Clynes et al., PNAS. 95:652-656, 1998.

Certain modified Fc regions may have altered stability or half-life relative to a corresponding, wild-type Fc sequence. In certain embodiments, such Fc regions may have increased half-life relative to a corresponding, wild-type Fc sequence. In other embodiments, modified Fc regions may have decreased half-life relative to a corresponding, wild-type Fc sequence. Half-life can be measured in vitro (e.g., under physiological conditions) or in vivo, according to routine techniques in the art, such as radiolabeling, ELISA, or other methods. In vivo measurements of stability or half-life can be measured in one or more bodily fluids, including blood, serum, plasma, urine, or cerebrospinal fluid, or a given tissue, such as the liver, kidneys, muscle, central nervous system tissues, bone, etc. As one example, modifications to an Fc region that alter its ability to bind the FcRn can alter its half-life in vivo. Assays for measuring the in vivo pharmacokinetic properties (e.g., in vivo mean elimination half-life) and non-limiting examples of Fc modifications that alter its binding to the FcRn are described, for example, in U.S. Pat. Nos. 7,217,797 and 7,732,570; and U.S. Application Nos. US 2010/0143254 and 2010/0143254.

In certain aspects, the Fc region may be modified by phosphorylation, sulfation, acrylation, glycosylation, methylation, farnesylation, acetylation, amidation, and the like, for instance, relative to a wild-type or naturally-occurring or commercial Fc region. In certain embodiments, the Fc region may comprise increased glycosylation relative to a native form, decreased glycosylation relative to a native form, or it may be entirely deglycosylated. As one example of a modified Fc glycoform, decreased glycosylation of an Fc region reduces binding to the C1q region of the first complement component C1, a decrease in ADCC-related activity, and/or a decrease in CDC-related activity. Certain embodiments thus employ a deglycosylated or aglycosylated Fc region. See, e.g., WO 2005/047337 for the production of exemplary aglycosylated Fc regions. Another example of an Fc region glycoform can be generated by substituting the Q295 position with a cysteine residue (see, e.g., U.S. Application No. 2010/0080794), according to the Kabat et al. numbering system. Certain embodiments may include Fc regions where about 80-100% of the glycoprotein in Fc region comprises a mature core carbohydrate structure that lacks fructose (see, e.g., U.S. Application No. 2010/0255013).

Specific examples of modified Fc regions having altered (e.g., increased, decreased) FcR binding can be found, for example, in U.S. Pat. Nos. 5,624,821 and 7,425,619; U.S. Application Nos. 2009/0017023, 2009/0010921, and 2010/0203046; and WO 2000/42072 and WO 2004/016750. Certain examples include human Fc regions having a one or more substitutions at position 298, 333, and/or 334, for example, 5298A, E333A, and/or K334A (based on the numbering of the EU index of Kabat et al.), which have been shown to increase binding to the activating receptor FcγRIIIa and reduce binding to the inhibitory receptor FcγRIIb. These mutations can be combined to obtain double and triple mutation variants that have further alterations in binding to FcRs. Certain embodiments include a S298A/E333A/K334A triple mutant, which has increased binding to FcγRIIIa, decreased binding to FcγRIIb, and increased ADCC (see, e.g., Shields et al., J Biol Chem. 276:6591-6604, 2001; and Presta et al., Biochem Soc Trans. 30:487-490, 2002). Some embodiments include Fc regions that comprise one or more substitutions selected from 434S, 252Y/428L, 252Y/434S, and 428L/434S (see U.S. Application Nos. 2009/0163699 and 20060173170), based on the EU index of Kabat et al.

Additional examples include modified IgG Fc regions having conservative or non-conservative substitutions (as described elsewhere herein) at one or more of positions 250, 314, or 428 of the heavy chain, or in any combination thereof, such as at positions 250 and 428, or at positions 250 and 314, or at positions 314 and 428, or at positions 250, 314, and 428 (see, e.g., U.S. Application No. 2011/0183412). In specific embodiments, the residue at position 250 is substituted with glutamic acid or glutamine, and/or the residue at position 428 is substituted with leucine or phenylalanine. As another illustrative example of an IgG Fc variant, any one or more of the amino acid residues at positions 214 to 238, 297 to 299, 318 to 322, and/or 327 to 331 may be used as a suitable target for modification (e.g., conservative or non-conservative substitution, deletion). In particular embodiments, the IgG Fc variant CH₂ domain contains amino acid substitutions at positions 228, 234, 235, and/or 331 (e.g., human IgG4 with Ser228Pro and Leu235Ala mutations) to attenuate the effector functions of the Fc region (see U.S. Pat. No. 7,030,226). Here, the numbering of the residues in the heavy chain is that of the EU index (see Kabat et al., “Sequences of Proteins of Immunological Interest,” 5^{th} Ed., National Institutes of Health, Bethesda, Md. (1991)). Certain of these and related embodiments have altered (e.g., increased, decreased) FcRn binding and/or serum half-life, optionally with reduced effector functions such as ADCC or CDC-related activities.

Additional examples include modified Fc regions that comprise one or more amino acid substitutions at positions 279, 341, 343 or 373 of a wild-type Fc region, or any combination thereof (see, e.g., U.S. Application No. 2007/0224188). The wild-type amino acid residues at these positions for human IgG are valine (279), glycine (341), proline (343) and tyrosine (373). The substation(s) can be conservative or non-conservative, or can include non-naturally occurring amino acids or mimetics, as described herein. Alone or in combination with these substitutions, certain embodiments may also employ a variant Fc region that comprises at least 1, 2, 3, 4, 5, 6, 7, 8, 9, 10 or more amino acid substitutions selected from the following: 235G, 235R, 236F, 236R, 236Y, 237K, 237N, 237R, 238E, 238G, 238H, 238I, 238L, 238V, 238W, 238Y, 244L, 245R, 247A, 247D, 247E, 247F, 247M, 247N, 247Q, 247R, 247S, 247T, 247W, 247Y, 248F, 248P, 248Q, 248W, 249L, 249M, 249N, 249P, 249Y, 251H, 2511, 251W, 254D, 254E, 254F, 254G, 254H, 254I, 254K, 254L, 254M, 254N, 254P, 254Q, 254R, 254V, 254W, 254Y, 255K, 255N, 256H, 2561, 256K, 256L, 256V, 256W, 256Y, 257A, 257I, 257M, 257N, 257S, 258D, 260S, 262L, 264S, 265K, 265S, 267H, 2671, 267K, 268K, 269N, 269Q, 271T, 272H, 272K, 272L, 272R, 279A, 279D, 279F, 279G, 279H, 2791, 279K, 279L, 279M, 279N, 279Q, 279R, 279S, 279T, 279W, 279Y, 280T, 283F, 283G, 283H, 283I, 283K, 283L, 283M, 283P, 283R, 283T, 283W, 283Y, 285N, 286F, 288N, 288P, 292E, 292F, 292G, 2921, 292L, 293S, 293V, 301W, 304E, 307E, 307M, 312P, 315F, 315K, 315L, 315P, 315R, 316F, 316K, 317P, 317T, 318N, 318P, 318T, 332F, 332G, 332L, 332M, 332S, 332V, 332W, 339D, 339E, 339F, 339G, 339H, 339I, 339K, 339L, 339M, 339N, 339Q, 339R, 339S, 339W, 339Y, 341D, 341E, 341F, 341H, 341I, 341K, 341L, 341M, 341N, 341P, 341Q, 341R, 341S, 341T, 341V, 341W, 341Y, 343A, 343D, 343E, 343F, 343G, 343H, 343I, 343K, 343L, 343M, 343N, 343Q, 343R, 343S, 343T, 343V, 343W, 343Y, 373D, 373E, 373F, 373G, 373H, 373I, 373K, 373L, 373M, 373N, 373Q, 373R, 373S, 373T, 373V, 373W, 375R, 376E, 376F, 376G, 376H, 376I, 376L, 376M, 376N, 376P, 376Q, 376R, 376S, 376T, 376V, 376W, 376Y, 377G, 377K, 377P, 378N, 379N, 379Q, 379S, 379T, 380D, 380N, 380S, 380T, 382D, 382F, 382H, 382I, 382K, 382L, 382M, 382N, 382P, 382Q, 382R, 382S, 382T, 382V, 382W, 382Y, 385E, 385P, 386K, 423N, 424H, 424M, 424V, 426D, 426L, 427N, 429A, 429F, 429M, 430A, 430D, 430F, 430G, 430H, 430I, 430K, 430L, 430M, 430N, 430P, 430Q, 430R, 430S, 430T, 430V, 430W, 430Y, 431H, 431K, 431P, 432R, 432S, 438G, 438K, 438L, 438T, 438W, 439E, 439H, 439Q, 440D, 440E, 440F, 440G, 440H, 440I, 440K, 440L, 440M, 440Q, 440T, 440V or 442K. As above, the numbering of the residues in the heavy chain is that of the EU index (see Kabat et al., supra). Such variant Fc regions typically confer an altered effector function or altered serum half-life. Preferably the altered effector function is a decrease in ADCC, a decrease in CDC, a decrease in C1q binding affinity, and/or a decrease in FcR (preferably FcRn) binding affinity relative to a corresponding Fc region that lacks such amino acid substitution(s).

Further examples include variant Fc regions that comprise one or more of the following amino acid substitutions: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, optionally where the variant has altered recognition of an Fc ligand and/or altered effector function compared with a parent Fc polypeptide, and wherein the numbering of the residues is that of the EU index as in Kabat et al. Specific examples of these and related embodiments include variant Fc regions that comprise or consist of the following sets of substitutions: (1) N276D, R2920, V305A, I377V, T394A, V412A and K439E; (2) P244L, K246E, D399G and K409R; (3) 5304G, K320R, S324T, K326E and M358T; (4) F243S, P247L, D265V, V266A, S383N and T411I; (5) H224N, F243L, T393A and H433P; (6) V240A, S267G, G341E and E356G; (7) M252T, P291L, P352A, R355W, N390D, S408G, S426F and A431S; (8) P228L, T289A, L3650, N389S and S440G; (9) F241L, V273A, K340Q and L441F; (10) F241L, T299A, I332T and M428T; (11) E269K, Y300H, Q342R, V422I and G446A; (12) T225A, R301c, S304G, D312N, N315D, L351S and N421S; (13) S254T, L306I, K326R and Q362L; (14) H224Y, P230S, V323A, E333D, K338R and S364C; (15) T335I, K414M and P445R; (16) T335I and K414M; (17) P247A, E258K, D280N, K288R, N297D, T299A, K322E, Q342R, S354A and L365P; (18) H268N, V279M, A339T, N361D and S426P; (19) C261Y, K290E, L306F, Q311R, E333G and Q438L; (20) E283G, N315K, E333G, R344Q, L365P and S442T; (21) Q347R, N361Y and K439R; (22) S239P, S254P, S267N, H285R, N3155, F372L, A378T, N390D, Y391C, F404S, E430K, L432P and K447E; and (23) E269G, Y278H, N325S and K370R, wherein the numbering of the residues is that of the EU index as in Kabat et al. (see, e.g., U.S. Application No. 2010/0184959).

Another specific example of an Fc variant comprises the sequence of Xaa Glu Ser Lys Tyr Gly Pro Pro Cys Pro Pro Cys Pro Ala Pro Xaa Xaa Xaa Gly Gly Pro Ser Val Phe Leu Phe Pro Pro Lys Pro Lys Asp Thr Leu Met Ile Ser Arg Thr Pro Glu Val Thr Cys Val Val Val Asp Val Ser Gln Glu Asp Pro Glu Val Gln Phe Asn Trp Tyr Val Asp Gly Val Glu Val His Asn Ala Lys Thr Lys Pro Arg Glu Glu Gln Phe Xaa Ser Thr Tyr Arg Val Val Ser Val Leu Thr Val Leu His Gln Asp Trp Leu Asn Gly Lys Glu Tyr Lys Cys Lys Val Ser Asn Lys Gly Leu Pro Ser Ser Ile Glu Lys Thr Ile Ser Lys Ala Lys Gly Gln Pro Arg Glu Pro Gln Val Tyr Thr Leu Pro Pro Ser Gln Glu Glu Met Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Thr Thr Pro Pro Val Leu Asp Ser Asp Gly Ser Phe Phe Leu Tyr Ser Arg Leu Thr Val Asp Lys Ser Arg Trp Gln Glu Gly Asn Val Phe Ser Cys Ser Val Met His Glu Ala Leu His Asn His Tyr Thr Gln Lys Ser Leu Ser Leu Ser Leu Gly Xaa (SEQ ID NO:225), wherein Xaa at position 1 is Ala or absent; Xaa at position 16 is Pro or Glu; Xaa at position 17 is Phe, Val, or Ala; Xaa at position 18 is Leu, Glu, or Ala; Xaa at position 80 is Asn or Ala; and/or Xaa at position 230 is Lys or is absent (see, e.g., U.S. Application No. 2007/0253966). Certain of these Fc regions have increased half-life, reduced effector activity, and/or are significantly less immunogenic than wild-type Fc sequences.

Variant Fc regions can also have one or more mutated hinge regions, as described, for example, in U.S. Application No. 2003/0118592. For instance, one or more cysteines in a hinge region can be deleted or substituted with a different amino acid. The mutated hinge region can comprise no cysteine residues, or it can comprise 1, 2, or 3 fewer cysteine residues than a corresponding, wild-type hinge region. In some embodiments, an Fc region having a mutated hinge region of this type exhibits a reduced ability to dimerize, relative to a wild-type Ig hinge region. In some aspects, all or a portion of the hinge region is deleted. In some embodiments, the hinge region is unaltered.

Also included are hybrid Fc regions, for example, Fc regions that comprise a combination of Fc domains (e.g., hinge, CH₂, CH₃, CH₄) from immunoglobulins of different species, different Ig classes, and/or different Ig subclasses. Preferably, such hybrid Fc regions have altered (e.g., reduced) binding to one or more Fc receptors or other Fc ligands described herein, and/or altered (e.g., reduced) altered effector functions, relative to a corresponding, wild-type Fc sequence. General examples include hybrid Fc regions that comprise, consist of, or consist essentially of the following combination of CH₂/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgE/IgA1, IgE/IgA2, IgE/IgD, IgE/IgE, IgE/IgG1, IgE/IgG2, IgE/IgG3, IgE/IgG4, IgE/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM, IgM/IgA1, IgM/IgA2, IgM/IgD, IgM/IgE, IgM/IgG1, IgM/IgG2, IgM/IgG3, IgM/IgG4, IgM/IgM (or fragments or variants thereof), and optionally include a hinge from one or more of IgA1, IgA2, IgD, IgG1, IgG2, IgG3, or IgG4, and/or a CH₄ domain from IgE and/or IgM. In specific embodiments, the hinge, CH₂, CH₃, and CH₄ domains are from human Ig.

Additional examples include hybrid Fc regions that comprise, consist of, or consist essentially of the following combination of CH₂/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₃ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Certain examples include hybrid Fc regions that comprise, consist of, or consist essentially of the following combination of CH₃/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₂ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Particular examples include hybrid Fc regions that comprise, consist of, or consist essentially of the following combination of hinge/CH₂ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₃ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Certain examples include hybrid Fc regions that comprise, consist of, or consist essentially of the following combination of hinge/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₂ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Some examples include hybrid Fc regions that comprise, consist of, or consist essentially of the following combination of hinge/CH₄ domains: IgA1/IgE, IgA1/IgM, IgA2/IgE, IgA2/IgM, IgD/IgE, IgD/IgM, IgG1/IgE, IgG1/IgM, IgG2/IgE, IgG2/IgM, IgG3/IgE, IgG3/IgM, IgG4/IgE, IgG4/IgM (or fragments or variants thereof), and optionally include a CH₂ domain from one or more of IgA1, IgA2, IgD, IgE, IgG1, IgG2, IgG3, IgG4, or IgM, and/or a CH₃ domain from one or more of IgA1, IgA2, IgD, IgE, IgG1, IgG2, IgG3, IgG4, or IgM.

Specific examples of hybrid Fc regions can be found, for example, in WO 2008/147143, which are derived from combinations of IgG subclasses or combinations of human IgD and IgG.

In some embodiments, the modified Fc region increases the accumulation of the conjugate in CNS tissues (e.g., brain parenchyma) by about or at least about 2, 3, 4, 5, 6, 7, 8, 9, 10, 11, 12, 13, 14, 15, 16, 17, 18, 19, 20, 25, 30, 40, or 50-fold relative to a corresponding unmodified or differently modified conjugate upon administration to a mammal. In some embodiments, the modified Fc region increases the rate of accumulation of the conjugate in CNS tissues by about or at least about 2, 3, 4, 5, 6, 7, 8, 9, 10, 11, 12, 13, 14, 15, 16, 17, 18, 19, 20, 25, 30, 40, or 50-fold relative to a corresponding unmodified or differently modified conjugate upon administration to a mammal. The increased accumulation (or rate of accumulation) of the conjugate in CNS tissues can be measured, for example, at one or more of about 5, 10, 15, 20, 30, 40, 50, 60, 90, 120 minutes or more post-administration, or at one or more of about 1, 2, 3, 4, 5, 6, 7, 8, 9, 10, 11, 12, 16, 24, 36, 48, 60, 72, 84, or 96 hours or more post-administration, or at one or more of about 1, 2, 3, 4, 5, 6, 7 or more days post-administration.

In certain embodiments, the conjugate having a modified Fc region has substantially the same secondary structure as a corresponding unmodified or differently modified conjugate, as determined, for example, via UV circular dichroism analysis. In certain embodiments, the conjugate having a modified Fc region has substantially the same biological activity of a corresponding unmodified or differently modified conjugate in a suitable in vitro assay or in vivo.

Antibodies

In certain embodiments, the conjugate comprises an antibody having a modified Fc region, as described herein. The antibody used in the conjugates or compositions of the present invention can be of essentially any type. Particular examples include therapeutic and diagnostic antibodies. As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule.

As used herein, the term “antibody” encompasses not only intact polyclonal or monoclonal antibodies, but also fragments thereof (such as dAb, Fab, Fab′, F(ab′)₂, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity.

The term “antigen-binding fragment” as used herein refers to a polypeptide fragment that contains at least one CDR of an immunoglobulin heavy and/or light chains that binds to the antigen of interest. In this regard, an antigen-binding fragment of the herein described antibodies may comprise 1, 2, 3, 4, 5, or all 6 CDRs of a VH and VL sequence from antibodies that bind to a therapeutic or diagnostic target.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. Epitopes can be contiguous or non-contiguous in relation to the primary structure of the antigen.

A molecule such as an antibody is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}.

Immunological binding properties of selected antibodies and polypeptides can be quantified using methods well known in the art (see Davies et al., Annual Rev. Biochem. 59:439-473, 1990). In some embodiments, an antibody or other polypeptide is said to specifically bind an antigen or epitope thereof when the equilibrium dissociation constant is about ≦10⁻⁷ or 10⁻⁸ M. In some embodiments, the equilibrium dissociation constant of an antibody may be about ≦10⁻⁹ M or ≦10⁻¹⁰ M. In certain illustrative embodiments, an antibody or other polypeptide has an affinity (K_{d}) for an antigen or target described herein (to which it specifically binds) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In certain embodiments, the antibody specifically binds to a host protein or antigen that is associated with a neuropathology (e.g., neuroinflammatory condition), including any one or more neuroinflammatory and/or auto-immune-associated antigens. In particular embodiments, the antibody specifically binds to a host protein or antigen that is associated with a neuroinflammatory condition. In some aspects, the host protein or antigen is a cell surface receptor or other cell surface protein. In some embodiments, the antigen is a ligand of a cell surface receptor or other cell surface protein. In certain embodiments, the antigen is an intracellular protein. In particular embodiments, the host protein or antigen is a human protein.

In some embodiments, the antibody specifically binds to an antigen associated with (e.g., treatment of) at least one nervous system disorder, including disorders of the peripheral and/or central nervous system (CNS) disorder. In certain embodiments, the antibody or other polypeptide specifically binds to an antigen associated with (e.g., treatment of) pain, including acute pain, chronic pain, and neuropathic pain. In some embodiments, the antibody specifically binds an antigen associated with (e.g., treatment of) an autoimmune disorder, including autoimmune disorders of the nervous system or CNS.

Examples of nervous system-, pain-, and/or autoimmune-associated antigens include, without limitation, alpha-4 (α4) integrin, tumor necrosis factor (TNF), IL-12, IL-23, the p40 subunit of IL-12 and IL-23, CD20, CD52, amyloid-β (e.g., Aβ₍₁₋₄₂₎), Huntingtin, CD25 (i.e., the alpha chain of the IL-2 receptor), nerve growth factor (NGF), neurotrophic tyrosine kinase receptor type 1 (TrkA; the high affinity catalytic receptor for NGF), and α-synuclein. In some embodiments, the antibody specifically binds at least one of the interleukin-2 (IL-2) receptor, α4 integrin, CD20, CD52, IL-12, IL-23, the p40 subunit of IL-12 and IL-23, or the axonal regrowth and remyelination inhibitors Nogo-A and LINGO. These targets have been considered useful in the treatment of a variety of nervous system, pain, and/or autoimmune disorders, such as multiple sclerosis (α4 integrin, IL-23, CD25, CD20, CD52, IL-12, IL-23, the p40 subunit of IL-12 and IL-23, Nogo-A, LINGO-1), Alzheimer's Disease (Aβ, TNF), Huntington's Disease (Huntingtin), Parkinson's Disease (α-synuclein), and pain (NGF and TrkA).

IL-2 Receptor.

In some aspects, the antibody specifically binds to the IL-2 receptor (IL-2R). The IL-2R is a heterotrimeric protein expressed on the surface of certain immune cells, such as lymphocytes, which binds and responds to the cytokine IL-2. The IL-2R has 3 non-covalently-associated subunits: a (CD25), β(CD122) and γ(CD132). The α and β subunits bind to IL-2, and the β and γ-subunits together facilitate signal transduction after said binding. The β and γ subunits of IL-2R are members of the type I cytokine receptor family.

Hence, in certain aspects, the antibody specifically binds to the alpha-subunit of the IL-2 receptor (CD25), and optionally reduces the interaction between IL2 and CD25. In some aspects, the antibody specifically binds to the beta-subunit of the IL-2 receptor (CD122), and optionally reduces the interaction between IL-2 and CD122, and/or reduces the signal transduction resulting from binding of IL-2 to IL-2R. In particular aspects, the antibody specifically binds to the gamma-subunit of the IL-2 receptor (CD132), and optionally reduces the signal transduction resulting from binding of IL-2 to IL-2R. In specific aspects, the antibody is daclizumab, or a variant or fragment thereof that specifically binds to CD25, and which has a modified Fc region, as described herein.

Alpha-4 Integrin.

In certain aspects, the antibody specifically binds to α4 integrin. Alpha-4 integrin (or CD49d) is one component of the integrin dimer Very Late Antigen-4 (VLA-4, or Integrin alpha4beta1), the other component being beta-1 integrin (CD29, or integrin beta-1). Unlike other integrin alpha chains, α4 integrin neither contains an I-domain, nor undergoes disulfide-linked cleavage. α4 integrin also associates with beta 7 chain. In some aspects, the antibody is natalizumab, or a variant or fragment thereof that specifically binds to α4 integrin, and which has a modified Fc region, as described herein.

CD20.

In some aspects, the antibody specifically binds to CD20. CD20 is an activated-glycosylated phosphoprotein expressed on the surface of all B-cells beginning at the pro-B phase (CD45R+, CD117+) and progressively increasing in concentration until maturity. Hence, in some embodiments, the antibody specifically binds to a CD20+ B-cell in a subject or in vitro. In specific embodiments, the antibody specifically binds to CD20, and has one or more of the following effects on a CD20+ B-cell: (a) mediates antibody-dependent cellular cytotoxicity (ADCC) and/or complement-dependent cytotoxicity (CDC) against the B-cell, (b) increases expression of MHC II, LFA-1, and/or LFA-3 (lymphocyte function-associated antigen), (c) increases shedding of CD23, (d) downregulates the B cell receptor, (e) induces apoptosis of the B-cell, or (e) any combination of the foregoing. In specific embodiments, the antibody is rituximab, tositumomab, ocrelizumab, or ofatumumab, TRU-015, or veltuzumab, or a variant or fragment thereof that specifically binds to CD20, and which has a modified Fc region, as described herein.

CD52.

In some aspects, the antibody specifically binds to CD52. CD52 is a glycosylphosphatidylinositol (GPI)-anchored antigen expressed on mature lymphocytes, monocytes, and certain dendritic cell subsets. In specific embodiments, the antibody is alemtuzumab, or a variant or fragment thereof that specifically binds to CD52, and which has a modified Fc region, as described herein.

IL-12.

In some embodiments, the antibody specifically binds to interleukin-12 (IL-12). IL-12 is an interleukin produced by dendritic cells, macrophages and human B-lymphoblastoid cells (NC-37) in response to antigenic stimulation. It is a heterodimeric cytokine encoded by two separate genes, IL-12A (p35) and IL-12B (p40).

IL-12 is involved in the differentiation of naive T cells into Th0 cells, which will further develop into either Th1 cells or Th2 cells. It is known as a T cell-stimulating factor, which can stimulate the growth and function of T cells. It stimulates the production of interferon-gamma (IFN-γ) and tumor necrosis factor-alpha (TNF-α) from T and natural killer (NK) cells, and reduces IL-4 mediated suppression of IFN-γ. IL-12 also mediates enhancement of the cytotoxic activity of NK cells and CD8+ cytotoxic T lymphocytes. IL-12 is associated with autoimmunity, which is believed to result from its role in the induction of Th1 immune responses. Hence, certain embodiments include antibodies that specifically bind to IL-12A (p35), IL-12B (p40), or both, and which optionally reduce or antagonize one or of the above-described biological activities of IL-12, including its role in the induction of Th1 immune responses, and which have a modified Fc region, as described herein.

IL-23.

In some embodiments, the antibody specifically binds to interleukin-23 (IL-23). IL-23 is a heterodimeric cytokine composed of two subunits, p40 and p19 (the IL-23 alpha subunit). In conjunction with IL-6 and TGF-β1, IL-23 stimulates naive CD4+ T cells to differentiate into Th17 cells, which are distinct from the classical Th1 and Th2 cells. Th17 cells produce IL-17, a pro-inflammatory cytokine that enhances T cell priming and stimulates the production of pro-inflammatory molecules such as IL-1, IL-6, TNF-alpha, NOS-2, and chemokines resulting in inflammation. Knockout mice deficient in either p40 or p19, or in either subunit of the IL-23 receptor (IL-23R and IL12R-β1) develop less severe symptoms of multiple sclerosis and inflammatory bowel disease. Certain embodiments thus include antibodies that specifically bind to p40, p19, or both, and which optionally reduce or antagonize one or of the above-described biological activities of IL-23, including its role in the production of Th17 cells, and which have a modified Fc region, as described herein

p40 Subunit of IL-12 and IL-23.

In some embodiments, the antibody specifically binds to the p40 subunit of IL-12 (i.e., IL-12 beta subunit) and IL-23. p40 (i.e., natural killer cell stimulatory factor 2, cytotoxic lymphocyte maturation factor 2) is a shared subunit of both IL-12 and IL-23. Overexpression of this gene has been observed in the central nervous system of patients with multiple sclerosis (MS), suggesting a role of this cytokine in the pathogenesis of the disease. Certain embodiments therefore relate to antibodies that specifically bind to p40, and which optionally reduce or antagonize one or more of the above-described activities of IL-12 and IL-23. In specific embodiments, the anti-p40 antibody is ustekinumab (CNTO 1275), or a variant or fragment thereof that specifically binds to the p40 subunit of IL-12 and IL-23, and which has a modified Fc region, as described herein.

Nogo-A.

Nogo-A is a splice isoform of Reticulon-4 (or Neurite outgrowth inhibitor; Nogo) and an axonal regrowth and remyelination inhibitor. Certain embodiments include antibodies that specifically bind to Nogo-A, and which optionally reduce or antagonize its activity as an axonal regrowth and remyelination inhibitor, that is, they increase axonal regrowth and remyelination in a subject, and which have a modified Fc region, as described herein.

LINGO-1.

LINGO-1 is a CNS-specific protein and a functional component of the NgR1/p75/LINGO-1 and NgR1/TAJ(TROY)/LINGO-1 signaling complexes that mediate inhibition of axonal outgrowth. These receptor complexes mediate the axonal growth inhibitory effects of Nogo, myelin-associated glycoprotein (MAG), and oligodendrocyte-myelin glycoprotein (OMgp) via RhoA activation. Certain embodiments include antibodies or antigen-binding fragments thereof that specifically bind to LINGO-1, and which optionally reduce or antagonize its activity as an axonal regrowth and remyelination inhibitor, that is, they increase axonal regrowth and remyelination in a subject.

In certain embodiments, the antibody specifically binds to a host protein or antigen that is associated with one or more types of pain, or a pain-associated antigen. In some aspects, the host protein or antigen is a cell surface receptor or other cell surface protein. In some embodiments, the antigen is a ligand of a cell surface receptor or other cell surface protein. In certain embodiments, the antigen is an intracellular protein. In particular embodiments, the host protein or antigen is a human protein. Exemplary pain-associated antigens include nerve growth factor (NGF) and neurotrophic tyrosine kinase receptor type 1 (TrkA).

Nerve Growth Factor.

NGF is a secreted protein contributes to the growth, maintenance, and survival of certain neurons, and is critical to the survival of sympathetic and sensory neurons. NGF forms a cysteine knot structure made up of beta strands twisted around each other and linked by disulfide bonds. Most structures are dimeric. NGF interacts with at least two classes of receptors: the p75 LNGFR (low affinity nerve growth factor receptor) neurotrophin receptor (p75(NTR)) and TrkA, a transmembrane tyrosine kinase. Both receptors are associated with neurodegenerative disorders.

NGF has also been shown to be a major mediator of pain, including inflammatory and neuropathic pain. For instance, preclinical animal models of inflammatory and neuropathic pain showed increased NGF levels, while the sequestration of NGF alleviated the associated hyperalgesia (see Watson et al., BioDrugs. 22:349-59, 2008). Certain embodiments thus include antibodies that specifically bind to NGF, and which optionally reduce its binding to TrkA, p75(NTR), or both, and which have a modified Fc region, as described herein. The identification of NGF epitopes that interact with TrkA can be facilitated by referring to the crystal structure of NGF in complex with the ligand-binding domain of TrkA (see Wiesmann et al., Nature. 401:184-188, 1999). In some embodiments, the antibody is tanezumab, or a variant or fragment thereof that specifically binds to NGF, and optionally reduces the binding between NGF and TrkA, and which has a modified Fc region, as described herein.

TrkA.

High affinity nerve growth factor receptor (neurotrophic tyrosine kinase receptor type 1; TRK1-transforming tyrosine kinase protein; TrkA) is a membrane-bound receptor kinase that upon binding to NGF, phosphorylates itself and members of the MAPK pathway. TrkA activation by NGF has been associated with pain, including inflammatory and neuropathic pain. Indeed, neutralizing antibodies directed against the TrkA receptor may display potent analgesic effects in inflammatory and chronic pain (see Ugolini et al., PNAS USA. 104:2985-2990, 2007).

TrkA is composed of an extracellular NGF-binding domain and an intracellular tyrosine kinase domain. Certain embodiments thus include antibodies that specifically bind to the extracellular NGF-binding domain of TrkA, and which optionally reduce the interaction between TrkA and NGF, and which have a modified Fc region, as described herein. The identification of TrkA epitopes that interact with NGF can be facilitated by referring to the crystal structure of NGF in complex with the ligand-binding domain of TrkA (see Wiesmann et al., supra) Other embodiments include antibodies or antigen-binding fragments thereof that specifically bind to the intracellular tyrosine kinase domain, and optionally inhibit the kinase activity of TrkA, for example, its autophosphorylation and/or its phosphorylation of members of the MAPK pathway. In specific embodiments, the antibody is MNAC13, or a variant or fragment thereof that specifically binds to TrkA, and which has a modified Fc region, as described herein.

In some embodiments, the antibody specifically binds to a pro-inflammatory molecule, for example, a pro-inflammatory cytokine or chemokine. In these and related embodiments, the conjugate can be used to treat a variety of inflammatory conditions, as described herein. Examples of pro-inflammatory molecules include tumor necrosis factors (TNF) such as TNF-α and TNF-β, TNF superfamily molecules such as FasL, CD27L, CD30L, CD40L, Ox40L, 4-1BBL, TRAIL, TWEAK, and Apo3L, interleukin-1 (IL-1) including IL-1a and IL-1β, IL-2, interferon-γ (IFN-γ), IFN-α/β, IL-6, IL-8, IL-12, IL-15, IL-17, IL-18, IL-21, LIF, CCL5, GROα, MCP-1, MIP-1α, MIP-1β, macrophage colony stimulating factor (MCSF), granulocyte macrophage colony stimulating factor (GM-CSF), CXCL2, CCL2, among others. In some embodiments, the antibody specifically binds to a receptor of one or more of the foregoing pro-inflammatory molecules, such as a TNF receptor (TNFR), an IL-1 receptor (IL-1R), or an IL-6 receptor (IL-6R), among others, and has a modified Fc region, as described herein.

In specific embodiments, as noted above, the antibody specifically binds to TNF-α or TNF-β. In particular embodiments, the anti-TNF antibody is adalimumab (Humira®), certolizumab pegol (Cimzia®), golimumab (Cimzia®), or infliximab (Remicade®), D2E7, CDP 571, or CDP 870, which has a modified Fc region, as described herein. Conjugates comprising an anti-TNF antibody can be used, for instance, in the treatment of various inflammatory conditions, as described herein. Such conjugates can also be used in the treatment of various neurological conditions or disorders such as Alzheimer's disease, stroke, traumatic brain injury (TBI), spinal stenosis, acute spinal cord injury, and spinal cord compression (see U.S. Pat. Nos. 6,015,557; 6,177,077; 6,419,934; 6,419,944; 6,537,549; 6,982,089; and 7,214,658).

In specific embodiments, the antibody specifically binds to IL-1α or IL-1β. In particular embodiments, the anti-IL-1 antibody is canakinumab or gevokizumab, or a variant or fragment thereof that specifically binds to IL-1β, and which has a modified Fc region, as described herein. Among other inflammatory conditions described herein, conjugates comprising an anti-IL-1 antibody can be used to treat cryopyrin-associated periodic syndromes (CAPS), including familial cold autoinflammatory syndrome, Muckle-Wells syndrome, and neonatal-onset multisystem inflammatory disease.

In some embodiments, the antibody specifically binds to a cancer-associated antigen, for instance, an antigen that is associated with a cancer of the central nervous system (CNS), i.e., a neurological cancer. In some instances, the antigen is associated with a metastatic cancer of the CNS, i.e., a metastatic brain cancer. Exemplary cancer antigens include cell surface proteins such as cell surface receptors. Also included as cancer-associated antigens are ligands that bind to such cell surface proteins or receptors. In specific embodiments, the antibody or antigen-binding fragment specifically binds to a intracellular cancer antigen. In some embodiments, the cancer that associates with the cancer antigen is one or more of breast cancer, metastatic brain cancer, prostate cancer, gastrointestinal cancer, lung cancer, ovarian cancer, testicular cancer, head and neck cancer, stomach cancer, bladder cancer, pancreatic cancer, liver cancer, kidney cancer, squamous cell carcinoma, CNS or brain cancer, melanoma, non-melanoma cancer, thyroid cancer, endometrial cancer, epithelial tumor, bone cancer, or a hematopoietic cancer.

Exemplary cancer-associated antigens include, without limitation, Her2/neu, B7H3, CD20, Her1/EGF receptor(s), VEGF receptor(s), PDGF receptor(s), CD30, CD52, CD33, CTLA-4, and tenascin. An additional example of a cancer-associated antigen is the B7H3 antigen (see Chen et al., Curr. Cancer Drug Targets. 8:404-413, 2008). In specific embodiments, the antibody is the 8H9 monoclonal antibody (see, e.g., Modak et al., Cancer Res. 61:4048-54, 2001), or an antigen-binding fragment thereof. In particular embodiments, the cancer-associated antigen, or cancer antigen, includes one or more of human Her2/neu, Her1/EGF receptor (EGFR), Her3, A33 antigen, CD5, CD19, CD20, CD22, CD23 (IgE Receptor), C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αᵥβ₃, integrin α₅β₁, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and/or mesothelin.

In specific embodiments, the antibody or antigen-binding fragment thereof or other polypeptide specifically binds to the human Her2/neu protein. Essentially any anti-Her2/neu antibody, antigen-binding fragment or other Her2/neu-specific binding agent may be used in producing the conjugates of the present invention. Illustrative anti-Her2/neu antibodies are described, for example, in U.S. Pat. Nos. 5,677,171; 5,720,937; 5,720,954; 5,725,856; 5,770,195; 5,772,997; 6,165,464; 6,387,371; and 6,399,063, the contents of which are incorporated herein by reference in their entireties.

In specific embodiments, the anti-Her2/neu antibody used in a conjugate is trastuzumab (Herceptin®), or a fragment, variant or derivative thereof which has a modified Fc region, as described herein. Herceptin® is a Her2/neu-specific monoclonal antibody approved for the treatment of human breast cancer. In certain embodiments, a Her2/neu-binding antigen-binding fragment comprises one or more of the CDRs of a Her2/neu antibody. In this regard, it has been shown in some cases that the transfer of only the VHCDR3 of an antibody can be performed while still retaining desired specific binding (Barbas et al., PNAS. 92: 2529-2533, 1995). See also, McLane et al., PNAS USA. 92:5214-5218, 1995; and Barbas et al., J. Am. Chem. Soc. 116:2161-2162, 1994.

In some embodiments, the antibody or antigen-binding fragment thereof or other polypeptide specifically binds to the human Her1/EGFR (epidermal growth factor receptor). Essentially any anti-Her1/EGFR antibody, antigen-binding fragment or other Her1-EGFR-specific binding agent may be used in producing the conjugates of the present invention. Illustrative anti-Her1/EGFR antibodies are described, for example, in U.S. Pat. Nos. 5,844,093; 7,132,511; 7,247,301; 7,595,378; 7,723,484; 7,939,072; and 7,960,516, the contents of which are incorporated by reference in their entireties.

In specific embodiments, the anti-Her1/EGFR antibody used in a conjugate of the invention is cetuximab (Erbitux®), or a fragment or derivative thereof which has a modified Fc region, as described herein. In certain embodiments, an anti-Her1/EGFR binding fragment comprises one or more of the CDRs of a Her1/EGFR antibody such as cetuximab. Cetuximab is approved for the treatment of head and neck cancer, and colorectal cancer. Cetuximab is composed of the Fv (variable; antigen-binding) regions of the 225 murine EGFR monoclonal antibody specific for the N-terminal portion of human EGFR with human IgG1 heavy and kappa light chain constant (framework) regions.

In certain embodiments, the antibody is a therapeutic antibody selected from trastuzumab, cetuximab, daclizumab, tanezumab, 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, cantuzumab (mertansine), cantuzumab (ravtansine), capromab (pendetide), catumaxomab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, rituximab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, tucotuzumab (celmoleukin), ublituximab, urelumab, veltuzumab, volociximab, votumumab, and zalutumumab, and which has a modified Fc region, as described herein.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)₂, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv or scFV antibodies are contemplated. For example, Kappa bodies (III et al., Prot. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (sFv) polypeptide is a covalently linked V_{H}::V_{L} heterodimer which is expressed from a gene fusion including V_{H}- and V_{L}-encoding genes linked by a peptide-encoding linker. Huston et al. (PNAS USA. 85(16):5879-5883, 1988). A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a “diabody.” Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). A dAb fragment of an antibody consists of a VH domain (Ward et al., Nature 341:544-546, 1989). Diabodies and other multivalent or multispecific fragments can be constructed, for example, by gene fusion (see WO94/13804; and Holliger et al., PNAS USA. 90:6444-6448, 1993)).

Minibodies comprising an scFv joined to a CH3 domain are also included (see Hu et al., Cancer Res. 56:3055-3061, 1996). See also Ward et al., Nature. 341:544-546, 1989; Bird et al., Science. 242:423-426, 1988; Huston et al., PNAS USA. 85:5879-5883, 1988); PCT/US92/09965; WO94/13804; and Reiter et al., Nature Biotech. 14:1239-1245, 1996.

Where bispecific antibodies are to be used, these may be conventional bispecific antibodies, which can be manufactured in a variety of ways (Holliger and Winter, Current Opinion Biotechnol. 4:446-449, 1993), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al., Protein Eng., 9:616-621, 1996).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies provided herein may take the form of a nanobody. Minibodies are encoded by single genes and are efficiently produced in almost all prokaryotic and eukaryotic hosts, for example, E. coli (see U.S. Pat. No. 6,765,087), moulds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells.

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

Fc-Fusion Polypeptides

In certain embodiments, the conjugate comprises an Fc-fusion polypeptide having a modified Fc region, as described herein. Typically, the Fc-fusion polypeptide is a therapeutic Fc-fusion polypeptide, optionally an existing and/or commercially-approved Fc-fusion polypeptide. Particular examples of Fc-fusion polypeptides include abatercept, aflibercept, alefacept, belatacept, etanercept, rilonacept, and romiplastin.

Abatercept blocks the interactions between CD80 or CD86 on antigen-presenting cells (APCs) and CD28 on T-cells, thereby inhibiting T-cell activation. It is currently approved in the United States for the treatment of rheumatoid arthritis. Abatercept is composed of the extracellular domain (ECD) of human cytotoxic T lymphocyte associated molecule-4 (CTLA-4) fused to human IgG1 Fc. Hence, certain conjugates comprise a BBB-transport moiety linked to abatercept, where the human IgG1 Fc region of abatercept is modified to alter (e.g., reduce) its binding to one or more Fc receptors/ligands.

Aflibercept binds to all forms of VEGF-A, as well as placental growth factor, thereby inhibiting angiogenesis. It is currently approved in the United States for the treatment of wet macular degeneration and metastatic colorectal cancer. Aflibercept is composed of the ECDs of VEGF receptors 1 and 2 fused to human IgG1 Fc. Certain conjugates thus comprise a BBB-transport moiety linked to aflibercept, where the human IgG1 Fc region of aflibercept is modified to alter (e.g., reduce) its binding to one or more Fc receptors/ligands.

Alefacept binds CD2, blocks the interactions between lymphocyte function-associated antigen (LFA) on APCs and CD2 on T-cells, thereby inhibiting T-cell activation. It is approved in the United States for the treatment of plaque psoriasis. Alefacept is composed of the first ECD of lymphocyte function-associated antigen 3 (LFA-3) fused to human IgG1 Fc. Certain conjugates therefore comprise a BBB-transport moiety linked to alefacept, where the human IgG1 Fc region of alefacept is modified to alter (e.g., reduce) its binding to one or more Fc receptors/ligands.

Belatacept blocks the interactions between CD80 or CD86 on APCs and CD28 on T-cells, thereby inhibiting T-cell activation. It is approved in the United States for prophylaxis of organ rejection in adults receiving a kidney transplant. Belatacept is composed of the ECD of CTLA-4 fused to human IgG1 Fc, and differs from abatacept by two amino acid substitutions (L104E, A29Y) in the CTLA-4 region. Certain conjugates comprise a BBB-transport moiety linked to belatacept, where the human IgG1 Fc region of belatacept is modified to alter (e.g., reduce) its binding to one or more Fc receptors/ligands.

Etanercept binds membrane-bound and soluble forms of TNF, thereby reducing concentrations of inflammatory cytokines. It is approved in the United States for the treatment of rheumatoid arthritis. Etanercept is composed of the 75 kDa soluble ECD of tumor necrosis factor (TNF) receptor II fused to human IgG1 Fc. Certain conjugates comprise a BBB-transport moiety linked to etanercept, where the human IgG1 Fc region of etanercept is modified to alter (e.g., reduce) its binding to one or more Fc receptors/ligands.

Rilonacept binds IL-1, thereby preventing its interaction with endogenous cell-surface receptors. It is approved in the United States for the treatment of plaque psoriasis. Rilonacept is composed of two chains, each comprising the C-terminus of the IL-1R accessory protein ligand binding region fused to the N-terminus of the IL-1RI ECD, fused to human IgG1 Fc. Certain conjugates thus comprise a BBB-transport moiety linked to rilonacept, where the human IgG1 Fc region of rilonacept is modified to alter (e.g., reduce) its binding to one or more Fc receptors/ligands.

Romiplastin binds to and agonizes the TPO receptor. It is approved in the United States for the treatment of thrombocytopenia. Romiplastin is composed of a peptide thrombopoietin (TPO) mimetic fused to the C-terminus of aglycosylated human IgG1 Fc. It is recombinantly produced in E. Coli and its Fc functionality is minimized due to lack of glycosylation. Certain conjugates thus comprise a BBB-transport moiety linked to romiplastin, where the human IgG1 Fc region of rilonacept is modified to further alter (e.g., further reduce or minimize) its binding to one or more Fc receptors/ligands.

Detectable Entities

In some embodiments, the CNS-targeted conjugate is further attached or linked to a “detectable entity.” Exemplary detectable entities include, without limitation, iodine-based labels, radioisotopes, fluorophores/fluorescent dyes, and nanoparticles.

Exemplary iodine-based labels include diatrizoic acid (Hypaque®, GE Healthcare) and its anionic form, diatrizoate. Diatrizoic acid is a radio-contrast agent used in advanced X-ray techniques such as CT scanning. Also included are iodine radioisotopes, described below.

Exemplary radioisotopes that can be used as detectable entities include ³²P, ³³P, ³⁵S, ³H, ¹³N, ¹⁵O, ¹¹¹In, ¹⁶⁹Yb, ^{99m}TC, ⁵⁵Fe, and isotopes of iodine such as ¹²³I, ¹²⁴I, ¹²⁵I, and ¹³¹I. These radioisotopes have different half-lives, types of decay, and levels of energy which can be tailored to match the needs of a particular protocol. Certain of these radioisotopes can be selectively targeted or better targeted to CNS tissues by conjugation to CNS-targeted conjugates, for instance, to improve the medical imaging of such tissues.

Examples of fluorophores or fluorochromes that can be used as directly detectable entities include fluorescein, tetramethylrhodamine, Texas Red, Oregon Green®, and a number of others (e.g., Haugland, Handbook of Fluorescent Probes—9th Ed., 2002, Molec. Probes, Inc., Eugene Oreg.; Haugland, The Handbook: A Guide to Fluorescent Probes and Labeling Technologies-10th Ed., 2005, Invitrogen, Carlsbad, Calif.). Also included are light-emitting or otherwise detectable dyes. The light emitted by the dyes can be visible light or invisible light, such as ultraviolet or infrared light. In exemplary embodiments, the dye may be a fluorescence resonance energy transfer (FRET) dye; a xanthene dye, such as fluorescein and rhodamine; a dye that has an amino group in the alpha or beta position (such as a naphthylamine dye, 1-dimethylaminonaphthyl-5-sulfonate, 1-anilino-8-naphthalende sulfonate and 2-p-touidinyl-6-naphthalene sulfonate); a dye that has 3-phenyl-7-isocyanatocoumarin; an acridine, such as 9-isothiocyanatoacridine and acridine orange; a pyrene, a bensoxadiazole and a stilbene; a dye that has 3-(ε-carboxypentyl)-3′-ethyl-5,5′-dimethyloxacarbocyanine (CYA); 6-carboxy fluorescein (FAM); 5&6-carboxyrhodamine-110 (R110); 6-carboxyrhodamine-6G (R6G); N,N,N′,N′-tetramethyl-6-carboxyrhodamine (TAMRA); 6-carboxy-X-rhodamine (ROX); 6-carboxy-4′,5′-dichloro-2′,7′-dimethoxyfluorescein (JOE); ALEXA FLUOR™; Cyt; Texas Red and Rhodamine Red; 6-carboxy-2′,4,7,7′-tetrachlorofluorescein (TET); 6-carboxy-2′,4,4′,5′,7,7′-hexachlorofluorescein (HEX); 5-carboxy-2′,4′,5′,7′-tetrachlorofluorescein (ZOE); NAN; NED; Cy3; Cy3.5; Cy5; Cy5.5; Cy7; and Cy7.5; IR800CW, ICG, Alexa Fluor 350; Alexa Fluor 488; Alexa Fluor 532; Alexa Fluor 546; Alexa Fluor 568; Alexa Fluor 594; Alexa Fluor 647; Alexa Fluor 680, or Alexa Fluor 750. Certain embodiments include conjugation to chemotherapeutic agents (e.g., paclitaxel, adriamycin) that are labeled with a detectable entity, such as a fluorophore (e.g., Oregon Green®, Alexa Fluor 488).

Nanoparticles usually range from about 1-1000 nm in size and include diverse chemical structures such as gold and silver particles and quantum dots. When irradiated with angled incident white light, silver or gold nanoparticles ranging from about 40-120 nm will scatter monochromatic light with high intensity. The wavelength of the scattered light is dependent on the size of the particle. Four to five different particles in close proximity will each scatter monochromatic light, which when superimposed will give a specific, unique color. Derivatized nanoparticles such as silver or gold particles can be attached to a broad array of molecules including, proteins, antibodies, small molecules, receptor ligands, and nucleic acids. Specific examples of nanoparticles include metallic nanoparticles and metallic nanoshells such as gold particles, silver particles, copper particles, platinum particles, cadmium particles, composite particles, gold hollow spheres, gold-coated silica nanoshells, and silica-coated gold shells. Also included are silica, latex, polystyrene, polycarbonate, polyacrylate, PVDF nanoparticles, and colored particles of any of these materials.

Quantum dots are fluorescing crystals about 1-5 nm in diameter that are excitable by light over a large range of wavelengths. Upon excitation by light having an appropriate wavelength, these crystals emit light, such as monochromatic light, with a wavelength dependent on their chemical composition and size. Quantum dots such as CdSe, ZnSe, InP, or InAs possess unique optical properties; these and similar quantum dots are available from a number of commercial sources (e.g., NN-Labs, Fayetteville, Ark.; Ocean Nanotech, Fayetteville, Ark.; Nanoco Technologies, Manchester, UK; Sigma-Aldrich, St. Louis, Mo.).

Polypeptide Variants and Fragments

Certain embodiments include variants and/or fragments of the reference polypeptides described herein, whether described by name or by reference to a sequence identifier. The wild-type or most prevalent or commercially-employed sequences of these polypeptides are known in the art, and can be used as a comparison for the variants and fragments described herein.

A polypeptide “variant,” as the term is used herein, is a polypeptide that typically differs from a polypeptide specifically disclosed herein by one or more substitutions, deletions, additions and/or insertions. Variant polypeptides are biologically active, that is, they continue to possess the enzymatic or binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence according to Table A below.

TABLE A

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In a preferred embodiment, variant polypeptides differ from a native sequence by substitution, deletion or addition of fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, a polypeptide sequence is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700. 700, 710, 720, 730, 740, 750, 760, 770, 780, 790, 800. 800, 810, 820, 830, 840, 850, 860, 870, 880, 890, 900, 900, 910, 920, 930, 940, 950, 960, 970, 980, 990, 1000 or more contiguous amino acids in length, including all integers in between, and which may comprise all or a portion of a reference sequence (see, e.g., Sequence Listing).

In other specific embodiments, a polypeptide sequence consists of about or no more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800. 800, 810, 820, 830, 840, 850, 860, 870, 880, 890, 900, 900, 910, 920, 930, 940, 950, 960, 970, 980, 990, 1000 or more contiguous amino acids, including all integers in between, and which may comprise all or a portion of a reference sequence (see, e.g., Sequence Listing).

In still other specific embodiments, a polypeptide sequence is about 10-1000, 10-900, 10-800, 10-700, 10-600, 10-500, 10-400, 10-300, 10-200, 10-100, 10-50, 10-40, 10-30, 10-20, 20-1000, 20-900, 20-800, 20-700, 20-600, 20-500, 20-400, 20-300, 20-200, 20-100, 20-50, 20-40, 20-30, 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, or 200-300 contiguous amino acids, including all ranges in between, and comprises all or a portion of a reference sequence. In certain embodiments, the C-terminal or N-terminal region of any reference polypeptide may be truncated by about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, or 800 or more amino acids, or by about 10-50, 20-50, 50-100, 100-150, 150-200, 200-250, 250-300, 300-350, 350-400, 400-450, 450-500, 500-550, 550-600, 600-650, 650-700, 700-750, 750-800 or more amino acids, including all integers and ranges in between (e.g., 101, 102, 103, 104, 105), so long as the truncated polypeptide retains the binding properties and/or activity of the reference polypeptide. Typically, the biologically-active fragment has no less than about 1%, about 5%, about 10%, about 25%, or about 50% of an activity of the biologically-active reference polypeptide from which it is derived.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence. Moreover, sequences differing from the native or parent sequences by the addition (e.g., C-terminal addition, N-terminal addition, both), deletion, truncation, insertion, or substitution of about 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties or activities of a parent or reference polypeptide sequence are contemplated.

In some embodiments, variant polypeptides differ from reference sequence by at least one but by less than 50, 40, 30, 20, 15, 10, 8, 6, 5, 4, 3 or 2 amino acid residue(s). In other embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In one embodiment, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In one embodiment, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In one particular embodiment, the variant polypeptide comprises an amino acid sequence that can be optimally aligned with a reference polypeptide sequence (see, e.g., Sequence Listing) to generate a BLAST bit scores or sequence similarity scores of at least about 50, 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

Exemplary Methods for Conjugation

Conjugation or coupling of a BBB-transport moiety to an antibody or other agent of interest can be carried out using standard chemical, biochemical and/or molecular techniques. Indeed, it will be apparent how to make a CNS-targeted conjugate in light of the present disclosure using available art-recognized methodologies. Of course, it will generally be preferred when coupling the primary components of a conjugate of the present invention that the techniques employed and the resulting linking chemistries do not substantially disturb the desired functionality or activity of the individual components of the conjugate.

The particular coupling chemistry employed will depend upon the structure of the biologically active agent (e.g., antibody, Fc-based polypeptide), the potential presence of multiple functional groups within the biologically active agent, the need for protection/deprotection steps, chemical stability of the agent, and the like, and will be readily determined by one skilled in the art. Illustrative coupling chemistry useful for preparing the conjugates of the invention can be found, for example, in Wong (1991), “Chemistry of Protein Conjugation and Crosslinking”, CRC Press, Boca Raton, Fla.; and Brinkley “A Brief Survey of Methods for Preparing Protein Conjugates with Dyes, Haptens, and Crosslinking Reagents,” in Bioconjug. Chem., 3:2013, 1992. Preferably, the binding ability and/or activity of the conjugate is not substantially reduced as a result of the conjugation technique employed, for example, relative to the unconjugated antibody or other agent or the unconjugated BBB-transport moiety.

In certain embodiments, a BBB-transport moiety may be coupled to an antibody or other agent of interest either directly or indirectly. A direct reaction between a BBB-transport moiety and an antibody or other agent of interest is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to indirectly couple a BBB-transport moiety and an antibody or other agent of interest via a linker group, including non-peptide linkers and peptide linkers. A linker group can also function as a spacer to distance an agent of interest from the BBB-transport moiety in order to avoid interference with binding capabilities, targeting capabilities or other functionalities. A linker group can also serve to increase the chemical reactivity of a substituent on an agent, and thus increase the coupling efficiency. An increase in chemical reactivity may also facilitate the use of agents, or functional groups on agents, which otherwise would not be possible. The selection of releasable or stable linkers can also be employed to alter the pharmacokinetics of a conjugate. Illustrative linking groups include, for example, disulfide groups, thioether groups, acid labile groups, photolabile groups, peptidase labile groups and esterase labile groups. In other illustrative embodiments, the conjugates include linking groups such as those disclosed in U.S. Pat. No. 5,208,020 or EP Patent 0 425 235 B1, and Chari et al., Cancer Research. 52: 127-131, 1992. Additional exemplary linkers are described below.

In some embodiments, it may be desirable to couple more than one BBB-transport moiety to an antibody or other agent, or vice versa. For example, in certain embodiments, multiple BBB-transport moieties are coupled to one antibody or other agent, or alternatively, one or more BBB-transport moieties are conjugated to multiple antibodies or other agents. The BBB-transport moieties can be the same or different. Regardless of the particular embodiment, conjugates containing multiple BBB-transport moieties may be prepared in a variety of ways. For example, more than one polypeptide may be coupled directly to an agent, or linkers that provide multiple sites for attachment can be used. Any of a variety of known heterobifunctional crosslinking strategies can be employed for making conjugates of the invention. It will be understood that many of these embodiments can be achieved by controlling the stoichiometries of the materials used during the conjugation/crosslinking procedure.

In certain exemplary embodiments, a reaction between an agent comprising a succinimidyl ester functional group and a BBB-transport moiety comprising an amino group forms an amide linkage; a reaction between an agent comprising a oxycarbonylimidizaole functional group and a BBB-transport moiety comprising an amino group forms an carbamate linkage; a reaction between an agent comprising a p-nitrophenyl carbonate functional group and a BBB-transport moiety comprising an amino group forms an carbamate linkage; a reaction between an agent comprising a trichlorophenyl carbonate functional group and a BBB-transport moiety comprising an amino group forms an carbamate linkage; a reaction between an agent comprising a thio ester functional group and a BBB-transport moiety comprising an n-terminal amino group forms an amide linkage; a reaction between an agent comprising a proprionaldehyde functional group and a BBB-transport moiety comprising an amino group forms a secondary amine linkage.

In some exemplary embodiments, a reaction between an agent comprising a butyraldehyde functional group and a BBB-transport moiety comprising an amino group forms a secondary amine linkage; a reaction between an agent comprising an acetal functional group and a BBB-transport moiety comprising an amino group forms a secondary amine linkage; a reaction between an agent comprising a piperidone functional group and a BBB-transport moiety comprising an amino group forms a secondary amine linkage; a reaction between an agent comprising a methylketone functional group and a BBB-transport moiety comprising an amino group forms a secondary amine linkage; a reaction between an agent comprising a tresylate functional group and a BBB-transport moiety comprising an amino group forms a secondary amine linkage; a reaction between an agent comprising a maleimide functional group and a BBB-transport moiety comprising an amino group forms a secondary amine linkage; a reaction between an agent comprising a aldehyde functional group and a BBB-transport moiety comprising an amino group forms a secondary amine linkage; and a reaction between an agent comprising a hydrazine functional group and a BBB-transport moiety comprising an carboxylic acid group forms a secondary amine linkage.

In particular exemplary embodiments, a reaction between an agent comprising a maleimide functional group and a BBB-transport moiety comprising a thiol group forms a thio ether linkage; a reaction between an agent comprising a vinyl sulfone functional group and a BBB-transport moiety comprising a thiol group forms a thio ether linkage; a reaction between an agent comprising a thiol functional group and a BBB-transport moiety comprising a thiol group forms a di-sulfide linkage; a reaction between an agent comprising a orthopyridyl disulfide functional group and a BBB-transport moiety comprising a thiol group forms a di-sulfide linkage; and a reaction between an agent comprising an iodoacetamide functional group and a BBB-transport moiety comprising a thiol group forms a thio ether linkage.

In a specific embodiment, an amine-to-sulfhydryl crosslinker is used for preparing a conjugate. In one preferred embodiment, for example, the crosslinker is succinimidyl-4-(N-maleimidomethyl)cyclohexane-1-carboxylate (SMCC) (Thermo Scientific), which is a sulfhydryl crosslinker containing NHS-ester and maleimide reactive groups at opposite ends of a medium-length cyclohexane-stabilized spacer arm (8.3 angstroms). SMCC is a non-cleavable and membrane permeable crosslinker that can be used to create sulfhydryl-reactive, maleimide-activated agents (e.g., polypeptides, antibodies) for subsequent reaction with BBB-transport moiety sequences. NHS esters react with primary amines at pH 7-9 to form stable amide bonds. Maleimides react with sulfhydryl groups at pH 6.5-7.5 to form stable thioether bonds. Thus, the amine reactive NHS ester of SMCC cross-links rapidly with primary amines of an agent and the resulting sulfhydryl-reactive maleimide group is then available to react with cysteine residues of the BBB-transport peptide moiety to yield specific conjugates of interest.

In certain specific embodiments, the BBB-transport moiety sequence is modified to contain exposed sulfhydryl groups to facilitate crosslinking, e.g., to facilitate crosslinking to a maleimide-activated agent. In a more specific embodiment, the BBB-transport moiety sequence is modified with a reagent which modifies primary amines to add protected thiol sulfhydryl groups. In an even more specific embodiment, the reagent N-succinimidyl-S-acetylthioacetate (SATA) (Thermo Scientific) is used to produce thiolated BBB-transport moieties.

In other specific embodiments, a maleimide-activated agent is reacted under suitable conditions with a thiolated BBB-transport moiety to produce a conjugate of the present invention. It will be understood that by manipulating the ratios of SMCC, SATA, agent, and the BBB-transport moiety in these reactions it is possible to produce conjugates having differing stoichiometries, molecular weights and properties.

In still other illustrative embodiments, conjugates are made using bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The specific crosslinking strategies discussed herein are but a few of many examples of suitable conjugation strategies that may be employed in producing conjugates of the invention. It will be evident to those skilled in the art that a variety of other bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Particular embodiments may employ one or more aldehyde tags to facilitate conjugation between a BBB-transport moiety and an antibody or other agent (see U.S. Pat. Nos. 8,097,701 and 7,985,783, incorporated by reference). Here, enzymatic modification at a sulfatase motif of the aldehyde tag through action of a formylglycine generating enzyme (FGE) generates a formylglycine (FGly) residue. The aldehyde moiety of the FGly residue can then be exploited as a chemical handle for site-specific attachment of a moiety of interest to the polypeptide. In some aspects, the moiety of interest is a small molecule, peptoid, aptamer, or peptide mimetic. In some aspects, the moiety of interest is another polypeptide, such as an antibody.

Particular embodiments thus include a BBB-transport moiety or antibody or other polypeptide agent that comprises 1, 2, 3, 4, 5, 6, 7, 8, 9, 10 or more heterologous sulfatase motifs, where the motif comprises the following structure:
X₁Z₁X₂Z₂X₃ (SEQ ID NO:226)

where Z₁ is cysteine or serine; Z₂ is a proline or alanine residue; X₁ is present or absent and, when present, is any amino acid, where X₁ is preferably present when the heterologous sulfatase motif is at an N-terminus of the aldehyde tagged polypeptide; and X₂ and X₃ are each independently any amino acid.

Polypeptides with the above-described motif can be modified by an FGE enzyme to generate a motif having a FGly residue, which, as noted above, can then be used for site-specific attachment of an agent, such as a second polypeptide, for instance, via a linker moiety. Such modifications can be performed, for example, by expressing the sulfatase motif-containing polypeptide (e.g., BBB-transport peptide moiety, antibody) in a mammalian, yeast, or bacterial cell that expresses an FGE enzyme or by in vitro modification of isolated polypeptide with an isolated FGE enzyme (see Wu et al., PNAS. 106:3000-3005, 2009; Rush and Bertozzi, J. Am Chem Soc. 130:12240-1, 2008; and Carlson et al., J Biol Chem. 283:20117-25, 2008).

Hence, some embodiments include a BBB-transport moiety or polypeptide agent (e.g., antibody) that comprises 1, 2, 3, 4, 5, 6, 7, 8, 9, 10 or more heterologous sulfatase motifs having a formylglycine residue, where the motif comprises the following structure:
X₁(FGly)X₂Z₂X₃ (SEQ ID NO:227)

where FGly is a formylglycine residue; Z₂ is a proline or alanine residue; X₁ is present or absent and, when present, is any amino acid, where X₁ is preferably present when the heterologous sulfatase motif is at an N-terminus of the aldehyde tagged polypeptide; and X₂ and X₃ are each independently any amino acid.

In particular embodiments, X₁, X₂, and X₃ are each independently an aliphatic amino acid, a sulfur-containing amino acid or a polar, uncharged amino acid. For instance, X₁ can be L, M, V, S or T; and X₂, and/or X₃ can be independently S, T, A, V, G or C.

In some embodiments, the heterologous sulfatase motif(s) can be (a) less than 16 amino acid residues in length, including about 4, 5, 6, 7, 8, 9, 10, 11, 12, 13, 14, or 15 residues in length, (b) positioned at the N-terminus of the polypeptide, (c) positioned at the C-terminus of the polypeptide, (d) positioned at an internal site of an amino acid sequence native to the polypeptide, (e) positioned in a terminal loop of the polypeptide, (f) positioned at a site of post-translational modification of the polypeptide (e.g., glycosylation site), or any combination thereof.

Some embodiments relate to conjugates of (i) a sulfatase motif (or aldehyde tag)-containing BBB-transport moiety, and (ii) an agent (A) such as small molecule that is functionalized with an aldehyde reactive group, where (i) and (ii) are covalently linked via the FGly residue of the sulfatase motif and the aldehyde reactive group. Such conjugates can have one of the following general structures:
BBB-transport moiety-(FGly)-R₁-A

where R₁ is at least one aldehyde reactive linkage; and FGly is a formylglycine residue within a heterologous sulfatase motif.

Some embodiments relate to conjugates of (i) a sulfatase motif (or aldehyde tag)-containing BBB-transport moiety, and (ii) a polypeptide agent (pA) that is functionalized with an aldehyde reactive group, or vice versa, where (i) and (ii) are covalently linked via the FGly residue of the sulfatase motif and the aldehyde reactive group. Such conjugates can have one of the following general structures:
BBB-transport moiety-(FGly)-R₁-pA or BBB-transport peptide moiety -R₁-(FGly)pA

where R₁ is at least one aldehyde reactive linkage; and FGly is a formylglycine residue within a heterologous sulfatase motif.

The agent or non-aldehyde tag-containing polypeptide (e.g., antibody, BBB-transport moiety) can be functionalized with one or more aldehyde reactive groups such as aminooxy, hydrazide, and thiosemicarbazide, and then covalently linked to the aldehyde tag-containing polypeptide via the at least one FGly residue, to form an aldehyde reactive linkage. The attachment of an aminooxy functionalized agent (or non-aldehyde tag-containing polypeptide) creates an oxime linkage between the FGly residue and the functionalized agent (or non-aldehyde tag-containing polypeptide); attachment of a hydrazide-functionalized agent (or non-aldehyde tag-containing polypeptide) creates a hydrazine linkage between the FGly residue and the functionalized agent (or non-aldehyde tag-containing polypeptide); and attachment of a thiosemicarbazide-functionalized agent (or non-aldehyde tag-containing polypeptide) creates a hydrazine carbothiamide linkage between the FGly residue and the functionalized agent (or non-aldehyde tag-containing polypeptide). Hence, in these and related embodiments, R₁ can be a linkage that comprises a Schiff base, such as an oxime linkage, a hydrazine linkage, or a hydrazine carbothiamide linkage.

Certain embodiments include conjugates of (i) a sulfatase motif (or aldehyde tag)-containing BBB-transport moiety and (ii) a sulfatase motif (or aldehyde tag)-containing antibody or other polypeptide agent (A), where (i) and (ii) are covalently linked via their respective FGly residues, optionally via a bi-functionalized linker moiety or group. For instance, certain conjugates may comprise the following structure:
BBB-transport peptide moiety-(FGly)-R₁-L-R₂-(FGly)A

where R₁ and R₂ are the same or different aldehyde reactive linkage; L is a linker moiety, BBB-transport peptide moiety-(FGly) is a aldehyde-tag containing BBB-transport moiety, and (FGly)A is an aldehyde tag-containing agent, such as an antibody or other polypeptide-based agent.

Merely by way of illustration, in some embodiments, the at least one heterologous sulfatase motif can be at the C-terminus of the BBB-transport moiety and the N-terminus of the polypeptide-based agent. In other embodiments, the at least one heterologous sulfatase motif can be at the N-terminus of the BBB-transport moiety and the C-terminus of the polypeptide-based agent. In still other embodiments, the at least one heterologous sulfatase motif can be at the N-terminus of the BBB-transport moiety and the N-terminus of the polypeptide-based agent. In further embodiments, the at least one heterologous sulfatase motif can be at the C-terminus of the BBB-transport moiety and the C-terminus of the polypeptide-based agent. As noted above, the at least one heterologous motif can be at an internal position in the BBB-transport moiety and/or the polypeptide-based agent. Persons skilled in the art will recognize that other combinations are possible.

The aldehyde reactive linkages of R₁ and R₂ can be independently formed by any aldehyde reactive group that will form a covalent bond between (i) the formylglycine (FGly) residue of the aldehyde tag and (ii) a linker moiety that is functionalized with said aldehyde reactive group (e.g., a bi-functionalized linker with two aldehyde reactive groups, which can be the same or different). Examples of aldehyde reactive groups include aminooxy, hydrazide, and thiosemicarbazide groups, which will form Schiff-base containing linkages with a FGly residue, including oxime linkages, hydrazine linkages, and hydrazine carbothiamide linkages, respectively. Hence, R₁ and R₂ can be independently a linkage that comprises a Schiff base, such as an oxime linkage, a hydrazine linkage, or a hydrazine carbothiamide linkage.

In some embodiments, the aldehyde tag-containing BBB-transport moiety and the aldehyde tag-containing antibody or other agent are linked (e.g., covalently linked) via a multi-functionalized linker (e.g., bi-functionalized linker), the latter being functionalized with the same or different aldehyde reactive group(s). In these and related embodiments, the aldehyde reactive groups allow the linker to form a covalent bridge between the BBB-transport moiety and the agent via their respective FGly residues. Linker moieties include any moiety or chemical that can be functionalized and preferably bi- or multi-functionalized with one or more aldehyde reactive groups. Particular examples include peptides, water-soluble polymers, detectable entities, other therapeutic compounds (e.g., cytotoxic compounds), biotin/streptavidin moieties, and glycans (see Hudak et al., J Am Chem Soc. 133:16127-35, 2011). Specific examples of glycans (or glycosides) include aminooxy glycans, such as higher-order glycans composed of glycosyl N-pentenoyl hydroxamates intermediates (supra). Exemplary linkers are described herein, and can be functionalized with aldehyde reactive groups according to routine techniques in the art (see, e.g., Carrico et al., Nat Chem Biol. 3:321-322, 2007; and U.S. Pat. Nos. 8,097,701 and 7,985,783).

Conjugates can also be prepared by a various “click chemistry” techniques, including reactions that are modular, wide in scope, give very high yields, generate mainly inoffensive byproducts that can be removed by non-chromatographic methods, and can be stereospecific but not necessarily enantioselective (see Kolb et al., Angew Chem Int Ed Engl. 40:2004-2021, 2001). Particular examples include conjugation techniques that employ the Huisgen 1,3-dipolar cycloaddition of azides and alkynes, also referred to as “azide-alkyne cycloaddition” reactions (see Hein et al., Pharm Res. 25:2216-2230, 2008). Non-limiting examples of azide-alkyne cycloaddition reactions include copper-catalyzed azide-alkyne cycloaddition (CuAAC) reactions and ruthenium-catalyzed azide-alkyne cycloaddition (RuAAC) reactions.

CuAAC works over a broad temperature range, is insensitive to aqueous conditions and a pH range over 4 to 12, and tolerates a broad range of functional groups (see Himo et al, J Am Chem Soc. 127:210-216, 2005). The active Cu(I) catalyst can be generated, for example, from Cu(I) salts or Cu(II) salts using sodium ascorbate as the reducing agent. This reaction forms 1,4-substituted products, making it region-specific (see Hein et al., supra).

RuAAC utilizes pentamethylcyclopentadienyl ruthenium chloride [Cp*RuCl] complexes that are able to catalyze the cycloaddition of azides to terminal alkynes, regioselectively leading to 1,5-disubstituted 1,2,3-triazoles (see Rasmussen et al., Org. Lett. 9:5337-5339, 2007). Further, and in contrast to CuAAC, RuAAC can also be used with internal alkynes to provide fully substituted 1,2,3-triazoles.

Certain embodiments thus include BBB-transport peptide moieties that comprise at least one unnatural amino acid with an azide side-chain or an alkyne side-chain, including internal and terminal unnatural amino acids (e.g., N-terminal, C-terminal). Certain of these peptide moieties can be formed by in vivo or in vitro (e.g., cell-free systems) incorporation of unnatural amino acids that contain azide side-chains or alkyne side-chains. Exemplary in vivo techniques include cell culture techniques, for instance, using modified E. coli (see Travis and Schultz, The Journal of Biological Chemistry. 285:11039-44, 2010; and Deiters and Schultz, Bioorganic & Medicinal Chemistry Letters. 15:1521-1524, 2005), and exemplary in vitro techniques include cell-free systems (see Bundy, Bioconjug Chem. 21:255-63, 2010).

In some embodiments, a BBB-transport peptide moiety that comprises at least one unnatural amino acid with an azide side-chain is conjugated by azide-alkyne cycloaddition to an agent (or linker) that comprises at least one alkyne group, such as an antibody or other polypeptide agent that comprises at least one unnatural amino acid with an alkyne side-chain. In other embodiments, a BBB-transport moiety that comprises at least one unnatural amino acid with an alkyne side-chain is conjugated by azide-alkyne cycloaddition to an antibody or other polypeptide agent (or linker) that comprises at least one azide group, such as a polypeptide agent that comprises at least one unnatural amino acid with an azide side-chain. Hence, certain embodiments include conjugates that comprise a BBB-transport moiety covalently linked to an agent via a 1,2,3-triazole linkage.

Specific conjugates can be formed by the following CuAAC-based or RuAAC-based reactions, to comprise the following respective structures (I) or (II).

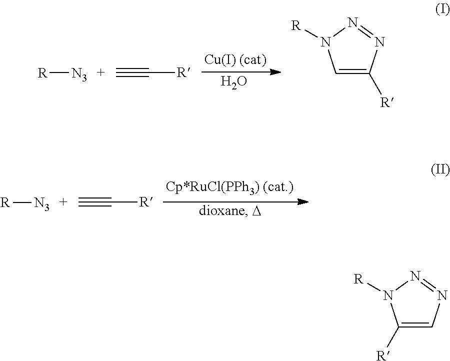

where R is a BBB-transport moiety and R^{I} is an agent of interest (or linker); or where R is an agent of interest (or linker) and R^{I} is a BBB-transport moiety.

In certain embodiments, the unnatural amino acid with the azide side-chain and/or the unnatural amino acid with alkyne side-chain are terminal amino acids (N-terminal, C-terminal). In certain embodiments, one or more of the unnatural amino acids are internal.

For instance, certain embodiments include a BBB-transport moiety that comprises an N-terminal unnatural amino acid with an azide side-chain conjugated to an agent that comprises an alkyne group. Some embodiments, include a BBB-transport moiety that comprises a C-terminal unnatural amino acid with an azide side-chain conjugated to an agent that comprises an alkyne group. Particular embodiments include a BBB-transport moiety that comprises an N-terminal unnatural amino acid with an alkyne side-chain conjugated to an agent that comprises an azide side-group. Further embodiments include a BBB-transport moiety that comprises an C-terminal unnatural amino acid with an alkyne side-chain conjugated to an agent that comprises an azide side-group. Some embodiments include a BBB-transport moiety that comprises at least one internal unnatural amino acid with an azide side-chain conjugated to an agent that comprises an alkyne group. Additional embodiments include a BBB-transport moiety that comprises at least one internal unnatural amino acid with an alkyne side-chain conjugated to an agent that comprises an azide side-group

Particular embodiments include a BBB-transport moiety that comprises an N-terminal unnatural amino acid with an azide side-chain conjugated to a polypeptide agent that comprises an N-terminal unnatural amino acid with an alkyne side-chain. Other embodiments include a BBB-transport moiety that comprises a C-terminal unnatural amino acid with an azide side-chain conjugated to a polypeptide agent that comprises a C-terminal unnatural amino acid with an alkyne side-chain. Still other embodiments include a BBB-transport moiety that comprises an N-terminal unnatural amino acid with an azide side-chain conjugated to a polypeptide agent that comprises a C-terminal unnatural amino acid with an alkyne side-chain. Further embodiments include a BBB-transport moiety that comprises a C-terminal unnatural amino acid with an azide side-chain conjugated to a polypeptide agent that comprises an N-terminal unnatural amino acid with an alkyne side-chain.

Other embodiments include a BBB-transport moiety that comprises an N-terminal unnatural amino acid with an alkyne side-chain conjugated to a polypeptide agent that comprises an N-terminal unnatural amino acid with an azide side-chain. Still further embodiments include a BBB-transport moiety that comprises a C-terminal unnatural amino acid with an alkyne side-chain conjugated to a polypeptide agent that comprises a C-terminal unnatural amino acid with an azide side-chain. Additional embodiments include a BBB-transport moiety that comprises an N-terminal unnatural amino acid with an alkyne side-chain conjugated to a polypeptide agent that comprises a C-terminal unnatural amino acid with an azide side-chain. Still further embodiments include a BBB-transport moiety that comprises a C-terminal unnatural amino acid with an alkyne side-chain conjugated to a polypeptide agent that comprises an N-terminal unnatural amino acid with an azide side-chain.

Also included are methods of producing a CNS-targeted conjugate, comprising: (a) performing an azide-alkyne cycloaddition reaction between (i) a BBB-transport moiety that comprises at least one unnatural amino acid with an azide side-chain and an agent that comprises at least one alkyne group (for instance, an unnatural amino acid with an alkyne side chain); or (ii) a BBB-transport moiety that comprises at least one unnatural amino acid with an alkyne side-chain and an agent that comprises at least one azide group (for instance, an unnatural amino acid with an azide side-chain); and (b) isolating a conjugate from the reaction, thereby producing a conjugate.

In the case where the conjugate is a fusion polypeptide, the fusion polypeptide may generally be prepared using standard techniques. Preferably, however, a fusion polypeptide is expressed as a recombinant polypeptide in an expression system, described herein and known in the art. Fusion polypeptides of the invention can contain one or multiple copies of a BBB-transport moiety and may contain one or multiple copies of a polypeptide-based agent of interest (e.g., antibody or antigen-binding fragment thereof), present in any desired arrangement.

For fusion proteins, DNA sequences encoding the BBB-transport moiety, the polypeptide agent (e.g., antibody), and optionally peptide linker components may be assembled separately, and then ligated into an appropriate expression vector. The 3′ end of the DNA sequence encoding one polypeptide component is ligated, with or without a peptide linker, to the 5′ end of a DNA sequence encoding the other polypeptide component(s) so that the reading frames of the sequences are in phase. The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the most C-terminal polypeptide. This permits translation into a single fusion polypeptide that retains the biological activity of both component polypeptides.

Similar techniques, mainly the arrangement of regulatory elements such as promoters, stop codons, and transcription termination signals, can be applied to the recombinant production of non-fusion proteins, for instance, BBB-transport moieties and polypeptide agents (e.g., antibody agents) for the production of non-fusion conjugates.

Polynucleotides and fusion polynucleotides of the invention can contain one or multiple copies of a nucleic acid encoding a BBB-transport moiety sequence, and/or may contain one or multiple copies of a nucleic acid encoding a polypeptide agent.

In some embodiments, a nucleic acids encoding a BBB-transport peptide moiety, antibody or other polypeptide agent, and/or a CNS-targeted fusion are introduced directly into a host cell, and the cell incubated under conditions sufficient to induce expression of the encoded polypeptide(s). The polypeptide sequences of this disclosure may be prepared using standard techniques well known to those of skill in the art in combination with the polypeptide and nucleic acid sequences provided herein.

Therefore, according to certain related embodiments, there is provided a recombinant host cell which comprises a polynucleotide or a fusion polynucleotide that encodes a polypeptide described herein. Expression of a conjugate (e.g., fusion protein conjugate), BBB-transport moiety, or antibody or other polypeptide agent in the host cell may conveniently be achieved by culturing under appropriate conditions recombinant host cells containing the polynucleotide. Following production by expression, the polypeptide(s) may be isolated and/or purified using any suitable technique, and then used as desired.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary (CHO) cells, HeLa cells, baby hamster kidney cells, HEK-293 cells, NSO mouse melanoma cells and many others. A common, preferred bacterial host is E. coli. The expression of polypeptides in prokaryotic cells such as E. coli is well established in the art. For a review, see for example Pluckthun, A. Bio/Technology. 9:545-551 (1991). Expression in eukaryotic cells in culture is also available to those skilled in the art as an option for recombinant production of polypeptides (see Ref, Curr. Opinion Biotech. 4:573-576, 1993; and Trill et al., Curr. Opinion Biotech. 6:553-560, 1995.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the polypeptides described herein, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Host cells may be chosen for certain characteristics, for instance, the expression of a formylglycine generating enzyme (FGE) to convert a cysteine or serine residue within a sulfatase motif into a formylglycine (FGly) residue, or the expression of aminoacyl tRNA synthetase(s) that can incorporate unnatural amino acids into the polypeptide, including unnatural amino acids with an azide side-chain, alkyne side-chain, or other desired side-chain, to facilitate conjugation.

Accordingly there is also contemplated a method comprising introducing such nucleic acid(s) into a host cell. The introduction of nucleic acids may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g. chromosome) of the host cell. Integration may be promoted by inclusion of sequences which promote recombination with the genome, in accordance—with standard techniques.

The present invention also provides, in certain embodiments, a method which comprises using a nucleic acid construct described herein in an expression system in order to express a particular polypeptide, such as an individual BBB-transport peptide moiety, antibody, or Fc-fusion polypeptide, or a conjugate as described herein.

As noted above, certain conjugates, such as fusion proteins, may employ one or more linker groups, including non-peptide linkers (e.g., non-proteinaceous linkers) and peptide linkers. Such linkers can be stable linkers or releasable linkers.

Exemplary non-peptide stable linkages include succinimide, propionic acid, carboxymethylate linkages, ethers, carbamates, amides, amines, carbamides, imides, aliphatic C—C bonds, thio ether linkages, thiocarbamates, thiocarbamides, and the like. Generally, a hydrolytically stable linkage is one that exhibits a rate of hydrolysis of less than about 1-2% to 5% per day under physiological conditions.

Exemplary non-peptide releasable linkages include carboxylate ester, phosphate ester, anhydride, acetal, ketal, acyloxyalkyl ether, imine, orthoester, thio ester, thiol ester, carbonate, and hydrazone linkages. Additional illustrative embodiments of hydrolytically unstable or weak linkages include, but are not limited to: —O₂C—(CH₂)_{b}—O—, where b is from 1 to 5, —O—(CH₂)_{b}—CO₂—(CH₂)_{c}—, where b is from 1 to 5 and c is from 2-5, —O—(CH₂)_{b}—CO₂—(CH₂)_{c}—O—, where b is from 1 to 5 and c is from 2-5, —(CH₂)_{b}—OPO₃—(CH₂)_{b′}—, where b is 1-5 and b′ is 1-5, —C(O)—(NH—CHR—CO)ₐ—NH—CHR—, where a is 2 to 20 and R is a substituent found on an α-amino acid, —O—(CH₂)_{b}—CO₂—CHCH₂—CH₂—, where b is from 1-5, —O—C₆H₄—CH═N—(CH₂)_{b}—O—, where b is from 1-5, and —O—(CH₂)_{b}—CH₂—CH═N—(CH₂)_{b}—O—, where each b is independently from 1-5.

Other illustrative examples of releasable linkers can be benzyl elimination-based linkers, trialkyl lock-based linkers (or trialkyl lock lactonization based), bicine-based linkers, and acid labile linkers. Among the acid labile linkers can be disulfide bond, hydrazone-containing linkers and thiopropionate-containing linkers.

Also included are linkers that are releasable or cleavable during or upon internalization into a cell. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.). In one embodiment, an acid-labile linker may be used (Cancer Research 52:127-131, 1992; and U.S. Pat. No. 5,208,020).

In certain embodiments, “water soluble polymers” are used in a linker for coupling a BBB-transport moiety to an agent of interest. A “water-soluble polymer” refers to a polymer that is soluble in water and is usually substantially non-immunogenic, and usually has an atomic molecular weight greater than about 1,000 Daltons. Attachment of two polypeptides via a water-soluble polymer can be desirable as such modification(s) can increase the therapeutic index by increasing serum half-life, for instance, by increasing proteolytic stability and/or decreasing renal clearance. Additionally, attachment via of one or more polymers can reduce the immunogenicity of protein pharmaceuticals. Particular examples of water soluble polymers include polyethylene glycol, polypropylene glycol, polyoxyalkylenes, or copolymers of polyethylene glycol, polypropylene glycol, and the like.

In some embodiments, the water-soluble polymer has an effective hydrodynamic molecular weight of greater than about 10,000 Da, greater than about 20,000 to 500,000 Da, greater than about 40,000 Da to 300,000 Da, greater than about 50,000 Da to 70,000 Da, usually greater than about 60,000 Da. The “effective hydrodynamic molecular weight” refers to the effective water-solvated size of a polymer chain as determined by aqueous-based size exclusion chromatography (SEC). When the water-soluble polymer contains polymer chains having polyalkylene oxide repeat units, such as ethylene oxide repeat units, each chain can have an atomic molecular weight of between about 200 Da and about 80,000 Da, or between about 1,500 Da and about 42,000 Da, with 2,000 to about 20,000 Da being of particular interest. Linear, branched, and terminally charged water soluble polymers are also included.

Polymers useful as linkers between aldehyde tagged polypeptides can have a wide range of molecular weights, and polymer subunits. These subunits may include a biological polymer, a synthetic polymer, or a combination thereof. Examples of such water-soluble polymers include: dextran and dextran derivatives, including dextran sulfate, P-amino cross linked dextrin, and carboxymethyl dextrin, cellulose and cellulose derivatives, including methylcellulose and carboxymethyl cellulose, starch and dextrines, and derivatives and hydroylactes of starch, polyalklyene glycol and derivatives thereof, including polyethylene glycol (PEG), methoxypolyethylene glycol, polyethylene glycol homopolymers, polypropylene glycol homopolymers, copolymers of ethylene glycol with propylene glycol, wherein said homopolymers and copolymers are unsubstituted or substituted at one end with an alkyl group, heparin and fragments of heparin, polyvinyl alcohol and polyvinyl ethyl ethers, polyvinylpyrrolidone, aspartamide, and polyoxyethylated polyols, with the dextran and dextran derivatives, dextrine and dextrine derivatives. It will be appreciated that various derivatives of the specifically described water-soluble polymers are also included.

Water-soluble polymers are known in the art, particularly the polyalkylene oxide-based polymers such as polyethylene glycol “PEG” (see Poly(ethylene glycol) Chemistry: Biotechnical and Biomedical Applications, J. M. Harris, Ed., Plenum Press, New York, N.Y. (1992); and Poly(ethylene glycol) Chemistry and Biological Applications, J. M. Harris and S. Zalipsky, Eds., ACS (1997); and International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966, incorporated by reference).

Exemplary polymers of interest include those containing a polyalkylene oxide, polyamide alkylene oxide, or derivatives thereof, including polyalkylene oxide and polyamide alkylene oxide comprising an ethylene oxide repeat unit of the formula —(CH₂—CH²—O)—. Further exemplary polymers of interest include a polyamide having a molecular weight greater than about 1,000 Daltons of the formula —[C(O)—X—C(O)—NH—Y—NH]ₙ— or —[NH—Y—NH—C(O)—X—C(O)]ₙ—, where X and Y are divalent radicals that may be the same or different and may be branched or linear, and n is a discrete integer from 2-100, usually from 2 to 50, and where either or both of X and Y comprises a biocompatible, substantially non-antigenic water-soluble repeat unit that may be linear or branched.

Further exemplary water-soluble repeat units comprise an ethylene oxide of the formula —(CH₂—CH₂—O)— or —(CH₂—CH₂—O)—. The number of such water-soluble repeat units can vary significantly, with the usual number of such units being from 2 to 500, 2 to 400, 2 to 300, 2 to 200, 2 to 100, and most usually 2 to 50. An exemplary embodiment is one in which one or both of X and Y is selected from: —((CH₂)ₙ₁—(CH₂—CH₂—O)ₙ₂—(CH₂)— or —((CH₂)ₙ₁—(O—CH₂—CH₂)ₙ₂—(CH₂)ₙ₁—), where n1 is 1 to 6, 1 to 5, 1 to 4 and most usually 1 to 3, and where n2 is 2 to 50, 2 to 25, 2 to 15, 2 to 10, 2 to 8, and most usually 2 to 5. A further exemplary embodiment is one in which X is —(CH₂—CH₂)—, and where Y is —(CH₂—(CH₂—CH₂—O)₃—CH₂—CH₂—CH₂)— or —(CH₂—CH₂—CH₂—(O—CH₂—CH₂)₃—CH₂)—, among other variations.

In certain embodiments, a peptide linker sequence may be employed to separate or couple the components of a conjugate. For instance, for polypeptide-polypeptide conjugates, peptide linkers can separate the components by a distance sufficient to ensure that each polypeptide folds into its secondary and tertiary structures. Such a peptide linker sequence may be incorporated into the conjugate (e.g., fusion protein) using standard techniques described herein and well-known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180.

In certain illustrative embodiments, a peptide linker is between about 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids. In other illustrative embodiments, a peptide linker comprises about 1, 5, 10, 15, 20, 25, 30, 35, 40, 45, 50 or more amino acids in length. Particular linkers can have an overall amino acid length of about 1-200 amino acids, 1-150 amino acids, 1-100 amino acids, 1-90 amino acids, 1-80 amino acids, 1-70 amino acids, 1-60 amino acids, 1-50 amino acids, 1-40 amino acids, 1-30 amino acids, 1-20 amino acids, 1-10 amino acids, 1-5 amino acids, 1-4 amino acids, 1-3 amino acids, or about 1, 2, 3, 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more amino acids.

A peptide linker may employ any one or more naturally-occurring amino acids, non-naturally occurring amino acid(s), amino acid analogs, and/or amino acid mimetics as described elsewhere herein and known in the art. Certain amino acid sequences which may be usefully employed as linkers include those disclosed in Maratea et al., Gene 40:39-46, 1985; Murphy et al., PNAS USA. 83:8258-8262, 1986; U.S. Pat. No. 4,935,233 and U.S. Pat. No. 4,751,180. Particular peptide linker sequences contain Gly, Ser, and/or Asn residues. Other near neutral amino acids, such as Thr and Ala may also be employed in the peptide linker sequence, if desired.

Certain exemplary linkers include Gly, Ser and/or Asn-containing linkers, as follows: [G]ₓ, [S]ₓ, [N]ₓ, [GS]ₓ, [GGS]ₓ, [GSS]ₓ, [GSGS]ₓ (SEQ ID NO:228), [GGSG]ₓ(SEQ ID NO:229), [GGGS]ₓ (SEQ ID NO: 230), [GGGGS]ₓ(SEQ ID NO:231), [GN]ₓ, [GGN]ₓ, [GNN]ₓ, [GNGN]ₓ(SEQ ID NO:232), [GGNG]ₓ(SEQ ID NO:233), [GGGN]ₓ (SEQ ID NO:234), [GGGGN]ₓ (SEQ ID NO:235) linkers, where ₓ is 1, 2, 3, 4, 5, 6, 7, 8, 9, 10, 11, 12, 13, 14, 15, 16, 17, 18, 19, or 20 or more. Other combinations of these and related amino acids will be apparent to persons skilled in the art.

In specific embodiments, the linker sequence comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

The peptide linkers may be physiologically stable or may include a releasable linker such as a physiologically degradable or enzymatically degradable linker (e.g., proteolytically cleavable linker). In certain embodiments, one or more releasable linkers can result in a shorter half-life and more rapid clearance of the conjugate. These and related embodiments can be used, for example, to enhance the solubility and blood circulation lifetime of conjugates in the bloodstream, while also delivering an agent into the bloodstream (or across the BBB) that, subsequent to linker degradation, is substantially free of the BBB-transport moiety. These aspects are especially useful in those cases where polypeptides or other agents, when permanently conjugated to a BBB-transport moiety, demonstrate reduced activity. By using the linkers as provided herein, such antibodies can maintain their therapeutic activity when in conjugated form. In these and other ways, the properties of the conjugates can be more effectively tailored to balance the bioactivity and circulating half-life of the antibodies over time.

Enzymatically degradable linkages suitable for use in particular embodiments of the present invention include, but are not limited to: an amino acid sequence cleaved by a serine protease such as thrombin, chymotrypsin, trypsin, elastase, kallikrein, or substilisin. Illustrative examples of thrombin-cleavable amino acid sequences include, but are not limited to: -Gly-Arg-Gly-Asp-(SEQ ID NO:236), -Gly-Gly-Arg-, -Gly-Arg-Gly-Asp-Asn-Pro-(SEQ ID NO:237), -Gly-Arg-Gly-Asp-Ser-(SEQ ID NO:238), -Gly-Arg-Gly-Asp-Ser-Pro-Lys-(SEQ ID NO:239), -Gly-Pro-Arg-, -Val-Pro-Arg-, and -Phe-Val-Arg-. Illustrative examples of elastase-cleavable amino acid sequences include, but are not limited to: -Ala-Ala-Ala-, -Ala-Ala-Pro-Val-(SEQ ID NO:240), -Ala-Ala-Pro-Leu-(SEQ ID NO:241), -Ala-Ala-Pro-Phe-(SEQ ID NO:242), -Ala-Ala-Pro-Ala-(SEQ ID NO:243), and -Ala-Tyr-Leu-Val-(SEQ ID NO:244).

Enzymatically degradable linkages suitable for use in particular embodiments of the present invention also include amino acid sequences that can be cleaved by a matrix metalloproteinase such as collagenase, stromelysin, and gelatinase. Illustrative examples of matrix metalloproteinase-cleavable amino acid sequences include, but are not limited to: -Gly-Pro-Y-Gly-Pro-Z-(SEQ ID NO:245), -Gly-Pro-, Leu-Gly-Pro-Z-(SEQ ID NO:246), -Gly-Pro-Ile-Gly-Pro-Z-(SEQ ID NO:247), and -Ala-Pro-Gly-Leu-Z-(SEQ ID NO:248), where Y and Z are amino acids. Illustrative examples of collagenase-cleavable amino acid sequences include, but are not limited to: -Pro-Leu-Gly-Pro-D-Arg-Z-(SEQ ID NO:249), -Pro-Leu-Gly-Leu-Leu-Gly-Z-(SEQ ID NO:250), -Pro-Gln-Gly-Ile-Ala-Gly-Trp-(SEQ ID NO:251), -Pro-Leu-Gly-Cys(Me)-His-(SEQ ID NO:252), -Pro-Leu-Gly-Leu-Tyr-Ala-(SEQ ID NO:253), -Pro-Leu-Ala-Leu-Trp-Ala-Arg-(SEQ ID NO:254), and -Pro-Leu-Ala-Tyr-Trp-Ala-Arg-(SEQ ID NO:255), where Z is an amino acid. An illustrative example of a stromelysin-cleavable amino acid sequence is -Pro-Tyr-Ala-Tyr-Tyr-Met-Arg-(SEQ ID NO:256); and an example of a gelatinase-cleavable amino acid sequence is -Pro-Leu-Gly-Met-Tyr-Ser-Arg-(SEQ ID NO:257).

Enzymatically degradable linkages suitable for use in particular embodiments of the present invention also include amino acid sequences that can be cleaved by an angiotensin converting enzyme, such as, for example, -Asp-Lys-Pro-, -Gly-Asp-Lys-Pro-(SEQ ID NO:258), and -Gly-Ser-Asp-Lys-Pro-(SEQ ID NO:259).

Enzymatically degradable linkages suitable for use in particular embodiments of the present invention also include amino acid sequences that can be degraded by cathepsin B, such as, for example, -Val-Cit-, -Ala-Leu-Ala-Leu- (SEQ ID NO:260), -Gly-Phe-Leu-Gly- (SEQ ID NO:261) and -Phe-Lys-.

In certain embodiments, however, any one or more of the non-peptide or peptide linkers are optional. For instance, linker sequences may not required in a fusion protein where the first and second polypeptides have non-essential N-terminal and/or C-terminal amino acid regions that can be used to separate the functional domains and prevent steric interference.

The functional properties of the conjugates described herein may be assessed using a variety of methods known to the skilled person, including, e.g., affinity/binding assays (for example, surface plasmon resonance, competitive inhibition assays); cytotoxicity assays, cell viability assays, cell proliferation or differentiation assays, cancer cell and/or tumor growth inhibition using in vitro or in vivo models. For instance, the conjugates described herein may be tested for effects on receptor internalization, in vitro and in vivo efficacy, etc., including the rate of transport across the blood brain barrier. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

Methods of Use and Pharmaceutical Compositions

Certain embodiments relate to methods of using the compositions of the conjugates described herein. Examples of such methods include methods of treatment and methods of diagnosis, including for instance, the use of conjugates for medical imaging of certain organs/tissues, such as those of the nervous system. Specific embodiments include methods of diagnosing and/or treating disorders or conditions of the central nervous system (CNS), or disorders or conditions having a CNS component.

Accordingly, certain embodiments include methods of treating a subject in need thereof, comprising administering a composition that comprises a conjugate described herein. Also included are methods of delivering an agent to the nervous system (e.g., central nervous system tissues) of a subject, comprising administering a composition that comprises a conjugate described herein. In certain of these and related embodiments, the methods increase the rate of delivery of the agent to the central nervous system tissues, relative, for example, to delivery by a composition that comprises the agent alone, or delivery by a composition that comprises a corresponding conjugate having an unmodified or differently modified Fc region.

In some instances, a subject has a disease, disorder, or condition that is associated with the central nervous system (CNS) or that has a CNS component, where increased delivery of a therapeutic antibody or other agent across the blood brain barrier to CNS tissues relative to peripheral tissues can improve treatment, for instance, by increasing the tissue concentration of the antibody or other agent in the CNS, and/or by reducing side-effects associated with exposure of the antibody or other agent to peripheral tissues/organs.

Certain embodiments relate to methods of treating inflammation or an inflammatory condition in a subject in need thereof, including inflammatory conditions of the CNS and/or those having a CNS component. “Inflammation” refers generally to the biological response of tissues to harmful stimuli, such as pathogens, damaged cells (e.g., wounds), and irritants. The term “inflammatory response” refers to the specific mechanisms by which inflammation is achieved and regulated, including, merely by way of illustration, immune cell activation or migration, cytokine production, vasodilation, including kinin release, fibrinolysis, and coagulation, among others described herein and known in the art. Ideally, inflammation is a protective attempt by the body to both remove the injurious stimuli and initiate the healing process for the affected tissue or tissues. In the absence of inflammation, wounds and infections would never heal, creating a situation in which progressive destruction of the tissue would threaten survival. On the other hand, excessive or chronic inflammation may associate with a variety of diseases, such as hay fever, atherosclerosis, and rheumatoid arthritis, among others described herein and known in the art.

Conjugates of the invention may modulate acute inflammation, chronic inflammation, or both. Depending on the needs of the subject, certain embodiments relate to reducing acute inflammation or inflammatory responses, and certain embodiments relate to reducing chronic inflammation or chronic inflammatory responses.

Acute inflammation relates to the initial response of the body to presumably harmful stimuli and involves increased movement of plasma and leukocytes from the blood into the injured tissues. It is a short-term process, typically beginning within minutes or hours and ending upon the removal of the injurious stimulus. Acute inflammation may be characterized by any one or more of redness, increased heat, swelling, pain, and loss of function. Redness and heat are due mainly to increased blood flow at body core temperature to the inflamed site, swelling is caused by accumulation of fluid, pain is typically due to release of chemicals that stimulate nerve endings, and loss of function has multiple causes.

Acute inflammatory responses are initiated mainly by local immune cells, such as resident macrophages, dendritic cells, histiocytes, Kuppfer cells and mastocytes. At the onset of an infection, burn, or other injuries, these cells undergo activation and release inflammatory mediators responsible for the clinical signs of inflammation, such as vasoactive amines and eicosanoids. Vasodilation and its resulting increased blood flow cause the redness and increased heat. Increased permeability of the blood vessels results in an exudation or leakage of plasma proteins and fluid into the tissue, which creates swelling. Certain released mediators such as bradykinin increase sensitivity to pain, and alter the blood vessels to permit the migration or extravasation of leukocytes, such as neutrophils, which typically migrate along a chemotactic gradient created by the local immune cells.

Acute inflammatory responses also includes one or more acellular biochemical cascade systems, consisting of preformed plasma proteins modulate, which act in parallel to initiate and propagate the inflammatory response. These systems include the complement system, which is mainly activated by bacteria, and the coagulation and fibrinolysis systems, which are mainly activated by necrosis, such as the type of tissue damage that is caused by certain infections, burns, or other trauma. Hence, conjugates may be used to modulate acute inflammation, or any of one or more of the individual acute inflammatory responses.

Chronic inflammation, a prolonged and delayed inflammatory response, is characterized by a progressive shift in the type of cells that are present at the site of inflammation, and often leads to simultaneous or near simultaneous destruction and healing of the tissue from the inflammatory process. At the cellular level, chronic inflammatory responses involve a variety of immune cells such as monocytes, macrophages, lymphocytes, plasma cells, and fibroblasts, though in contrast to acute inflammation, which is mediated mainly by granulocytes, chronic inflammation is mainly mediated by mononuclear cells such as monocytes and lymphocytes. Chronic inflammation also involves a variety of inflammatory mediators, such as IFN-γ and other cytokines, growth factors, reactive oxygen species, and hydrolytic enzymes. Chronic inflammation may last for many months or years, and may result in undesired tissue destruction and fibrosis.

Clinical signs of chronic inflammation are dependent upon duration of the illness, inflammatory lesions, cause and anatomical area affected. (see, e.g., Kumar et al., Robbins Basic Pathology-8th Ed., 2009 Elsevier, London; Miller, L M, Pathology Lecture Notes, Atlantic Veterinary College, Charlottetown, PEI, Canada). Chronic inflammation is associated with a variety of pathological conditions or diseases, including, for example, allergies, Alzheimer's disease, anemia, aortic valve stenosis, arthritis such as rheumatoid arthritis and osteoarthritis, cancer, congestive heart failure, fibromyalgia, fibrosis, heart attack, kidney failure, lupus, pancreatitis, stroke, surgical complications, inflammatory lung disease, inflammatory bowel disease, atherosclerosis, and psoriasis, among others described herein and known in the art. Hence, conjugates may be used to treat or manage chronic inflammation, modulate any of one or more of the individual chronic inflammatory responses, or treat any one or more diseases or conditions associated with chronic inflammation.

In certain embodiments, conjugates may modulate inflammatory responses at the cellular level, such as by modulating the activation, inflammatory molecule secretion (e.g., cytokine or kinin secretion), proliferation, activity, migration, or adhesion of various cells involved in inflammation. Examples of such cells include immune cells and vascular cells. Immune cells include, for example, granulocytes such as neutrophils, eosinophils and basophils, macrophages/monocytes, lymphocytes such as B-cells, killer T-cells (i.e., CD8+ T-cells), helper T-cells (i.e., CD4+ T-cells, including Tₕ1 and Tₕ2 cells), natural killer cells, γδ T-cells, dendritic cells, and mast cells. Examples of vascular cells include smooth muscle cells, endothelial cells, and fibroblasts. Also included are methods of modulating an inflammatory condition associated with one or more immune cells or vascular cells, including neutrophil-mediated, macrophage-mediated, and lymphocyte-mediated inflammatory conditions.

In certain embodiments, conjugates may modulate the levels or activity of inflammatory molecules, including plasma-derived inflammatory molecules and cell-derived inflammatory molecules. Included are pro-inflammatory molecules and anti-inflammatory molecules. Examples of plasma-derived inflammatory molecules include, without limitation, proteins or molecules of any one or more of the complement system, kinin system, coagulation system, and the fibrinolysis system. Examples of members of the complement system include C1, which exists in blood serum as a molecular complex containing about 6 molecules of C1q, 2 molecules of C1r, and 2 molecules of C1s, C2 (a and b), C3(a and B), C4 (a and b), C5, and the membrane attack complex of C5a, C5b, C6, C7, C8, and C9. Examples of the kinin system include bradykinin, kallidin, kallidreins, carboxypeptidases, angiotensin-converting enzyme, and neutral endopeptidase.

Examples of cell-derived inflammatory molecules include, without limitation, enzymes contained within lysosome granules, vasoactive amines, eicosanoids, cytokines, acute-phase proteins, and soluble gases such as nitric oxide. Vasoactive amines contain at least one amino group, and target blood vessels to alter their permeability or cause vasodilation. Examples of vasoactive amines include histamine and serotonin. Eicosanoids refer to signaling molecules made by oxidation of twenty-carbon essential fatty acids, and include prostaglandins, prostacyclins, thromboxanes, and leukotrienes.

Conjugates may also modulate levels or activity of acute-phase proteins. Examples of acute-phase proteins include C-reactive protein, serum amyloid A, serum amyloid P, and vasopressin. In certain instances, expression of acute-phase proteins can cause a range of undesired systemic effects including amyloidosis, fever, increased blood pressure, decreased sweating, malaise, loss of appetite, and somnolence. Accordingly, conjugates may modulate the levels or activity of acute-phase proteins, their systemic effects, or both.

In certain embodiments, conjugates reduce local inflammation, systemic inflammation, or both. In certain embodiments, conjugates may reduce or maintain (i.e., prevent further increases) local inflammation or local inflammatory responses. In certain embodiments, conjugates may reduce or maintain (i.e., prevent further increases) systemic inflammation or systemic inflammatory responses.

In certain embodiments, the modulation of inflammation or inflammatory responses can be associated with one or more tissues or organs. Non-limiting examples of such tissues or organs include skin (e.g., dermis, epidermis, subcutaneous layer), hair follicles, nervous system (e.g., brain, spinal cord, peripheral nerves, meninges including the dura mater, arachnoid mater, and pia mater), auditory system or balance organs (e.g., inner ear, middle ear, outer ear), respiratory system (e.g., nose, trachea, lungs), gastroesophogeal tissues, the gastrointestinal system (e.g., mouth, esophagus, stomach, small intestines, large intestines, rectum), vascular system (e.g., heart, blood vessels and arteries), liver, gallbladder, lymphatic/immune system (e.g., lymph nodes, lymphoid follicles, spleen, thymus, bone marrow), uro-genital system (e.g., kidneys, ureter, bladder, urethra, cervix, Fallopian tubes, ovaries, uterus, vulva, prostate, bulbourethral glands, epidiymis, prostate, seminal vesicles, testicles), musculoskeletal system (e.g., skeletal muscles, smooth muscles, bone, cartilage, tendons, ligaments), adipose tissue, mammaries, and the endocrine system (e.g., hypothalamus, pituitary, thyroid, pancreas, adrenal glands). Accordingly, conjugates may be used to modulate inflammation associated with any of these tissues or organs, such as to treat conditions or diseases that are associated with the inflammation of these tissues or organs.

In particular embodiments, the inflammatory condition has a nervous system or central nervous system component, including inflammation of the brain, spinal cord, and/or the meninges. In particular embodiments, the inflammatory condition of the CNS in meningitis (e.g., bacteria, viral), encephalitis (e.g., caused by infection or autoimmune inflammation such as Acute Disseminated Enchephalomyelitis), sarcoidosis, non-metastatic diseases associated with neoplasia. Particular examples of nervous system or CNS associated inflammatory conditions include, without limitation, meningitis (i.e., inflammation of the protective membranes covering the brain and spinal cord), myelitis, encaphaloymyelitis (e.g., myalgic encephalomyelitis, acute disseminated encephalomyelitis, encephalomyelitis disseminata or multiple sclerosis, autoimmune encephalomyelitis), arachnoiditis (i.e., inflammation of the arachnoid, one of the membranes that surround and protect the nerves of the central nervous system), granuloma, drug-induced inflammation or meningitis, neurodegenerative diseases such as Alzheimer's disease, stroke, HIV-dementia, encephalitis such viral encephalitis and bacterial encephalitis, parasitic infections, inflammatory demyelinating disorders, and auto-immune disorders such as CD8+ T Cell-mediated autoimmune diseases of the CNS. Additional examples include Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, stiff-man syndrome, stroke, traumatic brain injury (TBI), spinal stenosis, acute spinal cord injury, and spinal cord compression.

In some instances, the subject has multiple sclerosis (MS). MS is a chronic, neurological, autoimmune, demyelinating disease. MS can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent.

The pathology of MS is characterized by an abnormal immune response directed against the central nervous system. In particular, T-lymphocytes are activated against the myelin sheath of the neurons of the central nervous system causing demyelination. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most subjects recover clinically from individual bouts of demyelination, producing the classic remitting and exacerbating course of the most common form of the disease known as relapsing-remitting multiple sclerosis.

Diagnosis of MS can be made by brain and spinal cord magnetic resonance imaging (MRI), analysis of somatosensory evoked potentials, and analysis of cerebrospinal fluid to detect increased amounts of immunoglobulin or oligoclonal bands. MRI is a particularly sensitive diagnostic tool. MRI abnormalities indicating the presence or progression of MS include hyperintense white matter signals on T2-weighted and fluid attenuated inversion recovery images, gadolinium enhancement of active lesions, hypointensive “black holes” (representing gliosis and axonal pathology), and brain atrophy on T1-weighted studies. Serial MRI studies can be used to indicate disease progression.

Certain embodiments therefore methods of treating multiple sclerosis in a subject in need thereof, comprising administering to the subject a conjugate described herein. In specific instances, the subject has relapsing remitting MS, secondary progressive MS, primary progressive MS, or progressive relapsing MS. The relapsing-remitting subtype of MS is characterized by unpredictable relapses followed by periods of months to years of relative quiet (i.e., remission) with no new signs of disease activity. Secondary progressive MS refers to the progression from relapsing-remitting MS towards the occurrence of neurologic decline between acute attacks with little or no definite periods of remission. The primary progressive subtype of MS is characterized by progression of disability from onset with little or no remissions and improvements. Progressive relapsing MS is characterized by a steady neurologic decline on combination with clear superimposed attacks.

Certain embodiments include combination therapies for treating MS. For instance, a subject with MS may be administered a conjugate described herein, where the antibody specifically binds to at least one MS-associated antigen and has a modified Fc region as described herein, in combination with one or more MS therapeutic agents, including those used to manage the symptoms of MS. Exemplary MS therapeutic agents include, without limitation, interferon beta-1a, e.g., Avonex™, Rebif™, CinnoVex™), interferon beta-1b (e.g., Betaseron™), glatiramer acetate (e.g., Copaxone™), mitoxantrone (e.g., Novantrone™), fingolimod (Gilenya™), methotrexate, azathioprine, intravenous immunoglobulin (IVIg), cyclophosphamide, steroids, lioresal, tizanidine, benzodiazepines, cholinergics, antidepressants, and amantadine.

As noted above, also included is inflammation associated with infections of the nervous system or CNS. Specific examples of bacterial infections associated with inflammation of the nervous system include, without limitation, streptococcal infection such as group B streptococci (e.g., subtypes III) and Streptococcus pneumoniae (e.g., serotypes 6, 9, 14, 18 and 23), Escherichia coli (e.g., carrying K1 antigen), Listeria monocytogenes (e.g., serotype IVb), neisserial infection such as Neisseria meningitidis (meningococcus), staphylococcal infection, heamophilus infection such as Haemophilus influenzae type B, Klebsiella, and Mycobacterium tuberculosis. Also included are infections by staphylococci and pseudomonas and other Gram-negative bacilli, mainly with respect to trauma to the skull, which gives bacteria in the nasal cavity the potential to enter the meningeal space, or in persons with cerebral shunt or related device (e.g., extraventricular drain, Ommaya reservoir). Specific examples of viral infections associated with inflammation of the nervous system include, without limitation, enteroviruses, herpes simplex virus type 1 and 2, human T-lymphotrophic virus, varicella zoster virus (chickenpox and shingles), mumps virus, human immunodeficiency virus (HIV), and lymphocytic choriomeningitis virus (LCMV). Meningitis may also result from infection by spirochetes such as Treponema pallidum (syphilis) and Borrelia burgdorferi (Lyme disease), parasites such as malaria (e.g., cerebral malaria), fungi such as Cryptococcus neoformans, and ameoba such as Naegleria fowleri.

Meningitis or other forms of nervous system inflammation may also associate with the spread of cancer to the meninges (malignant meningitis), certain drugs such as non-steroidal anti-inflammatory drugs, antibiotics and intravenous immunoglobulins, sarcoidosis (or neurosarcoidosis), connective tissue disorders such as systemic lupus erythematosus, and certain forms of vasculitis (inflammatory conditions of the blood vessel wall) such as Behçet's disease. Epidermoid cysts and dermoid cysts may cause meningitis by releasing irritant matter into the subarachnoid space. Accordingly, conjugates may be used to treat or manage any one or more of these conditions.

In certain instances, the subject is experiencing one or more types of pain, and the conjugate is administered to treat or reduce the pain. General examples of pain include acute pain and chronic pain. In some instances, the pain has at least one CNS component. Specific examples of pain include nociceptive pain, neuropathic pain, breakthrough pain, incident pain, phantom pain, inflammatory pain including arthritic pain, or any combination thereof.

In particular instances, the pain is nociceptive pain, optionally visceral, deep somatic, or superficial somatic pain. Nociceptive pain is usually caused by stimulation of peripheral nerve fibers that respond to stimuli approaching or exceeding harmful intensity (nociceptors), and may be classified according to the mode of noxious stimulation; for example, “thermal” (e.g., heat or cold), “mechanical” (e.g., crushing, tearing, cutting) and “chemical.” Visceral structures are highly sensitive to stretch, ischemia and inflammation, but relatively insensitive to other stimuli such as burning and cutting. Visceral pain is most often diffuse, difficult to locate, and is sometimes referred to as having a distant, or superficial, structure. Visceral pain can be accompanied by nausea and vomiting, and is sometimes described as sickening, deep, squeezing, and dull. Deep somatic pain is usually initiated by the stimulation of nociceptors in ligaments, tendons, bones, blood vessels, fasciae and muscles, and is often characterized as a dull, aching, or poorly localized pain. Examples include sprains and broken bones. Superficial pain is mainly initiated by activation of nociceptors in the skin or other superficial tissue, and is sharp, well-defined and clearly located. Examples of injuries that produce superficial somatic pain include wounds and burns.

Neuropathic pain results from damage or disease affecting the somatosensory system. It may be associated with abnormal sensations called dysesthesia, and pain produced by normally non-painful stimuli (allodynia). Neuropathic pain may have continuous and/or episodic (paroxysmal) components, the latter being compared to an electric shock. Common characteristics of neuropathic pain include burning or coldness, “pins and needles” sensations, numbness, and itching. Neuropathic pain may result from disorders of the peripheral nervous system or the central nervous system (e.g., brain, spinal cord). Neuropathic pain may be characterized as peripheral neuropathic pain, central neuropathic pain, or mixed (peripheral and central) neuropathic pain.

Central neuropathic pain is found in spinal cord injury, multiple sclerosis, and strokes. Additional causes of neuropathic pain include diabetic neuropathy, herpes zoster infection, HIV-related neuropathies, nutritional deficiencies, toxins, remote manifestations of malignancies, immune mediated disorders, and physical trauma to a nerve trunk. Neuropathic pain also associates with cancer, mainly as a direct result of a cancer or tumor on peripheral or central nerves (e.g., compression by a tumor), or as a side effect of chemotherapy, radiation injury, or surgery.

In some instances, the pain is breakthrough pain. Breakthrough pain is pain that comes on suddenly for short periods of time and is not alleviated by the subject's normal pain management regimen. It is common in cancer patients who often have a background level of pain controlled by medications, but whose pain periodically “breaks through” the medication. Hence, in certain instances, the subject is taking pain medication, and is optionally a subject with cancer pain, e.g., neuropathic cancer pain.

In certain instances, the pain is incident pain, a type of pain that arises as a result of an activity. Examples include moving an arthritic or injured joint, and stretching a wound.

In specific instances, the pain is osteoarthritis, low back pain (or lumbago), including acute, sub-acute, and chronic low back pain (CLBP), bone cancer pain, or interstitial cystitis.

Osteoarthritis (OA), also referred to as degenerative arthritis or degenerative joint disease or osteoarthrosis, is a group of mechanical abnormalities involving degradation of joints, including articular cartilage and subchondral bone. Symptoms of OA may include joint pain, tenderness, stiffness, locking, and sometimes an effusion. OA may be initiated by variety of causes, including hereditary, developmental, metabolic, and mechanical causes, most of which lead to the loss of cartilage. When bone surfaces become less well protected by cartilage, bone may be exposed and damaged. As a result of decreased movement secondary to pain, regional muscles may atrophy, and ligaments may become increasingly lax. Particular examples include osteoarthritis of the knee, and osteoarthritis of the hip.

Interstitial cystitis, or bladder pain syndrome, is a chronic, oftentimes severely debilitating disease of the urinary bladder. Of unknown cause, it is characterized, for instance, by pain associated with the bladder, pain associated with urination (dysuria), urinary frequency (e.g., as often as every 10 minutes), urgency, and/or pressure in the bladder and/or pelvis.

Certain embodiments include combination therapies for treating pain. For instance, a subject with pain may be administered a conjugate described herein, where the antibody specifically binds to at least one pain-associated antigen, in combination with one or more pain medications, including analgesics and anesthetics. Exemplary analgesics include, without limitation, paracetamol/acetaminophen; non-steroidal anti-inflammatory drugs (NSAIDS) such as salicylates (e.g., aspirin), propionic acid derivatives (e.g., ibuprofen, naproxen), acetic acid derivatives (e.g., indomethacin), enolic acid derivatives, fenamic acid derivatives, and selective COX-2 inhibitors; opiates/opioids and morphinomimetics such as morphine, buprenorphine, codeine, oxycodone, oxymorphone, hydrocodone, dihydromorphine, dihydrocodeine, levorphanol, methadone, dextropropoxyphene, pentazocine, dextromoramide, meperidine (or pethidin), tramadol, noscapine, nalbuphine, pentacozine, papverine, papaveretum, alfentanil, fentanyl, remifentanil, sufentanil, and etorphine; and other agents, such as flupirtine, carbamazepine, gabapentin, and pregabalin, including any combination of the foregoing.

In some embodiments, conjugates may be used to treat various cancers, including cancers of the central nervous system (CNS), or neurological cancers. In some instances, the neurological cancer is a metastatic brain cancer. Examples of cancers that can metastasize to the brain include, without limitation, breast cancers, lung cancers, genitourinary tract cancers, gastrointestinal tract cancers (e.g., colorectal cancers, pancreatic carcinomas), osteosarcomas, melanomas, head and neck cancers, prostate cancers (e.g., prostatic adenocarcinomas), and lymphomas. Certain embodiments thus include methods for treating, inhibiting or preventing metastasis of a cancer by administering to a patient a therapeutically effective amount of a herein disclosed conjugate (e.g., in an amount that, following administration, inhibits, prevents or delays metastasis of a cancer in a statistically significant manner, i.e., relative to an appropriate control as will be known to those skilled in the art). In particular embodiments, the subject has a cancer that has not yet metastasized to the central nervous system, including one or more of the above-described cancers, among others known in the art.

Also included are methods for treating a cancer of the central nervous system (CNS), optionally the brain, where the subject in need thereof has such a cancer or is at risk for developing such a condition. In some embodiments, the cancer is a primary cancer of the CNS, such as a primary cancer of the brain. For instance, the methods can be for treating a glioma, meningioma, pituitary adenoma, vestibular schwannoma, primary CNS lymphoma, or primitive neuroectodermal tumor (medulloblastoma). In some embodiments, the glioma is an astrocytoma, oligodendroglioma, ependymoma, or a choroid plexus papilloma. In certain embodiments, the primary CNS or brain cancer is glioblastoma multiforme, such as a giant cell gliobastoma or a gliosarcoma.

In particular embodiments, the cancer is a metastatic cancer of the CNS, for instance, a cancer that has metastasized to the brain. Examples of such cancers include, without limitation, breast cancers, lung cancers, genitourinary tract cancers, gastrointestinal tract cancers (e.g., colorectal cancers, pancreatic carcinomas), osteosarcomas, melanomas, head and neck cancers, prostate cancers (e.g., prostatic adenocarcinomas), and lymphomas. Certain embodiments thus include methods for treating, inhibiting or preventing metastasis of a cancer by administering to a patient a therapeutically effective amount of a herein disclosed conjugate (e.g., in an amount that, following administration, inhibits, prevents or delays metastasis of a cancer in a statistically significant manner, i.e., relative to an appropriate control as will be known to those skilled in the art). In particular embodiments, the subject has a cancer that has not yet metastasized to the central nervous system, including one or more of the above-described cancers, among others known in the art.

In particular embodiments, the cancer (cell) expresses or overexpresses one or more of Her2/neu, B7H3, CD20, Her1/EGF receptor(s), VEGF receptor(s), PDGF receptor(s), CD30, CD52, CD33, CTLA-4, or tenascin.

Also included is the treatment of other cancers, including breast cancer, prostate cancer, gastrointestinal cancer, lung cancer, ovarian cancer, testicular cancer, head and neck cancer, stomach cancer, bladder cancer, pancreatic cancer, liver cancer, kidney cancer, squamous cell carcinoma, melanoma, non-melanoma cancer, thyroid cancer, endometrial cancer, epithelial tumor, bone cancer, or a hematopoietic cancer. Hence, in certain embodiments, the cancer cell being treated by a conjugate overexpresses or is associated with a cancer antigen, such as human Her2/neu, Her1/EGF receptor (EGFR), Her3, A33 antigen, B7H3, CD5, CD19, CD20, CD22, CD23 (IgE Receptor), C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αᵥβ₃, integrin α₅β₁, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and/or mesothelin.

In specific embodiments, the subject has a Her2/neu-expressing cancer, such as a breast cancer, ovarian cancer, stomach cancer, aggressive uterine cancer, or metastatic cancer, such as a metastatic CNS cancer, and the BBB-transport moiety is conjugated to trastuzumab, which has a modified Fc region, as described herein. In other specific embodiments, a 8H9 monoclonal antibody conjugate is used to treat a neurological cancer such as a metastatic brain cancer, where the antibody has a modified Fc region, as described herein.

The use of conjugates for treating cancers including cancers of the CNS can be combined with other therapeutic modalities. For example, a composition comprising a conjugate can be administered to a subject before, during, or after other therapeutic interventions, including symptomatic care, radiotherapy, surgery, transplantation, immunotherapy, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

In specific combination therapies, the antibody portion of a conjugate comprises cetuximab, and the conjugate is used for treating a subject with locally or regionally advanced squamous cell carcinoma of the head and neck in combination with radiation therapy. In other aspects, the cetuximab conjugate is used for treating a subject with recurrent locoregional disease or metastatic squamous cell carcinoma of the head and neck in combination with platinum-based therapy with 5-fluorouracil (5-FU). In some aspects, the cetuximab conjugate is used in combination with irinotecan for treating a subject with EGFR-expressing colorectal cancer and that is refractory to irinotecan-based chemotherapy.

Methods for identifying subjects with one or more of the diseases or conditions described herein are known in the art.

Also included are methods for imaging an organ or tissue component in a subject, comprising (a) administering to the subject a composition comprising a conjugate described herein, and (b) visualizing the detectable entity in the subject, organ, or tissue.

In particular embodiments, the organ or tissue compartment comprises the central nervous system (e.g., brain, brainstem, spinal cord). In specific embodiments, the organ or tissue compartment comprises the brain or a portion thereof, for instance, the parenchyma of the brain.

A variety of methods can be employed to visualize the detectable entity in the subject, organ, or tissue. Exemplary non-invasive methods include radiography, such as fluoroscopy and projectional radiographs, CT-scanning or CAT-scanning (computed tomography (CT) or computed axial tomography (CAT)), whether employing X-ray CT-scanning, positron emission tomography (PET), or single photon emission computed tomography (SPECT), and certain types of magnetic resonance imaging (MRI), especially those that utilize contrast agents, including combinations thereof.

Merely by way of example, PET can be performed with positron-emitting contrast agents or radioisotopes such as ¹⁸F, SPECT can be performed with gamma-emitting contrast agents or radioisotopes such as ²⁰¹TI, ^{99m}TC, ¹²³I, and ⁶⁷Ga, and MRI can be performed with contrast agents or radioisotopes such as ³H, ¹³C, ¹⁹F, ¹⁷O, ²³Na, ³¹P, and ¹²⁹Xe, and Gd (gadolidinium; chelated organic Gd (III) complexes). Any one or more of these exemplary contrast agents or radioisotopes can be conjugated to or otherwise incorporated into a conjugate and administered to a subject for imaging purposes. For instance, conjugates can be directly labeled with one or more of these radioisotopes, or conjugated to molecules (e.g., small molecules) that comprise one or more of these radioisotopic contrast agents, or any others described herein.

For in vivo use, for instance, for the treatment of human disease, medical imaging, or testing, the conjugates described herein are generally incorporated into a pharmaceutical composition prior to administration. A pharmaceutical composition comprises one or more of the BBB-transport moieties, polypeptides, peptides antibodies, detectable entities, or conjugates described herein in combination with a physiologically acceptable carrier or excipient.

To prepare a pharmaceutical composition, an effective or desired amount of one or more of the BBB-transport moieties, polypeptides, peptides antibodies, detectable entities, or conjugates is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

Administration of the conjugates and other agents described herein, in pure form or in an appropriate pharmaceutical composition, can be carried out via any of the accepted modes of administration of agents for serving similar utilities. The pharmaceutical compositions can be prepared by combining a polypeptide or conjugate or conjugate-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, subcutaneous or topical. Preferred modes of administration depend upon the nature of the condition to be treated or prevented.

Carriers can include, for example, pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In certain aspects, the BBB-transport moiety and the antibody or Fc-fusion polypeptide each, individually or as a pre-existing conjugate, bound to or encapsulated within a particle, e.g., a nanoparticle, bead, lipid formulation, lipid particle, or liposome, e.g., immunoliposome. For instance, in particular embodiments, the BBB-transport moiety is bound to the surface of a particle, and the antibody or other agent of interest is bound to the surface of the particle and/or encapsulated within the particle. In some of these and related embodiments, the BBB-transport moiety and the antibody or other agent are covalently or operatively linked to each other only via the particle itself (e.g., nanoparticle, liposome), and are not covalently linked to each other in any other way; that is, they are bound individually to the same particle. In other embodiments, the BBB-transport moiety and the antibody or other agent are first covalently or non-covalently conjugated to each other, as described herein (e.g., via a linker molecule), and are then bound to or encapsulated within a particle (e.g., immunoliposome, nanoparticle). In specific embodiments, the particle is a liposome, and the composition comprises one or more BBB-transport moieties, one or more antibodies or other agents of interest, and a mixture of lipids to form a liposome (e.g., phospholipids, mixed lipid chains with surfactant properties). In some aspects, the BBB-transport moiety and the antibody or other agent are individually mixed with the lipid/liposome mixture, such that the formation of liposome structures operatively links the BBB-transport moiety and the antibody or other agent without the need for covalent conjugation. In other aspects, the BBB-transport moiety and the antibody or other agent are first covalently or non-covalently conjugated to each other, as described herein, and then mixed with lipids to form a liposome. The BBB-transport moiety, the antibody or other agent, or the conjugate may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents, such as cytotoxic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described conjugate in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a BBB-transport moiety, agent, or conjugate described herein, for treatment of a disease or condition of interest.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a BBB-transport moiety, antibody or other agent, or conjugate as herein disclosed such that a suitable dosage will be obtained. Typically, this amount is at least 0.01% of the agent of interest in the composition. When intended for oral administration, this amount may be varied to be between 0.1 and about 70% of the weight of the composition. Certain oral pharmaceutical compositions contain between about 4% and about 75% of the agent of interest. In certain embodiments, pharmaceutical compositions and preparations according to the present invention are prepared so that a parenteral dosage unit contains between 0.01 to 10% by weight of the agent of interest prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the conjugate or agent and thereby assists in the delivery of the compound. Suitable agents that may act in this capacity include monoclonal or polyclonal antibodies, one or more proteins or a liposome.

The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the conjugates against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The pharmaceutical compositions may be prepared by methodology well known in the pharmaceutical art. For example, a pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the conjugate so as to facilitate dissolution or homogeneous suspension of the conjugate in the aqueous delivery system.

The compositions may be administered in a therapeutically effective amount, which will vary depending upon a variety of factors including the activity of the specific compound (e.g., conjugate) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g).

Compositions described herein may also be administered simultaneously with, prior to, or after administration of one or more other therapeutic agents, as described herein. For instance, in one embodiment, the conjugate is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Such combination therapy may include administration of a single pharmaceutical dosage formulation which contains a compound of the invention and one or more additional active agents, as well as administration of compositions comprising conjugates of the invention and each active agent in its own separate pharmaceutical dosage formulation. For example, a conjugate as described herein and the other active agent can be administered to the patient together in a single oral dosage composition such as a tablet or capsule, or each agent administered in separate oral dosage formulations. Similarly, a conjugate as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising conjugates and one or more additional active agents can be administered at essentially the same time, i.e., concurrently, or at separately staggered times, i.e., sequentially and in any order; combination therapy is understood to include all these regimens.

The various embodiments described herein can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1

### Effect of Fc Region on BBB Transport

Experiments were performed to compare the ability of p97 melanotransferrin (MTf) to transport across the blood brain barrier (BBB) an Fc-containing protein (etanercept) and a non-Fc-containing protein (a fragment antigen-binding region of an antibody (Fab fragment)). A p97 polypeptide (soluble p97) was conjugated to etanercept and a Fab of an anti-TNF-TNFR monoclonal antibody, and tested relative to unconjugated control proteins for distribution into brain tissues. For quantitative detection, all test proteins were labeled with Alexa Fluor 680 (AF680) according to routine techniques.

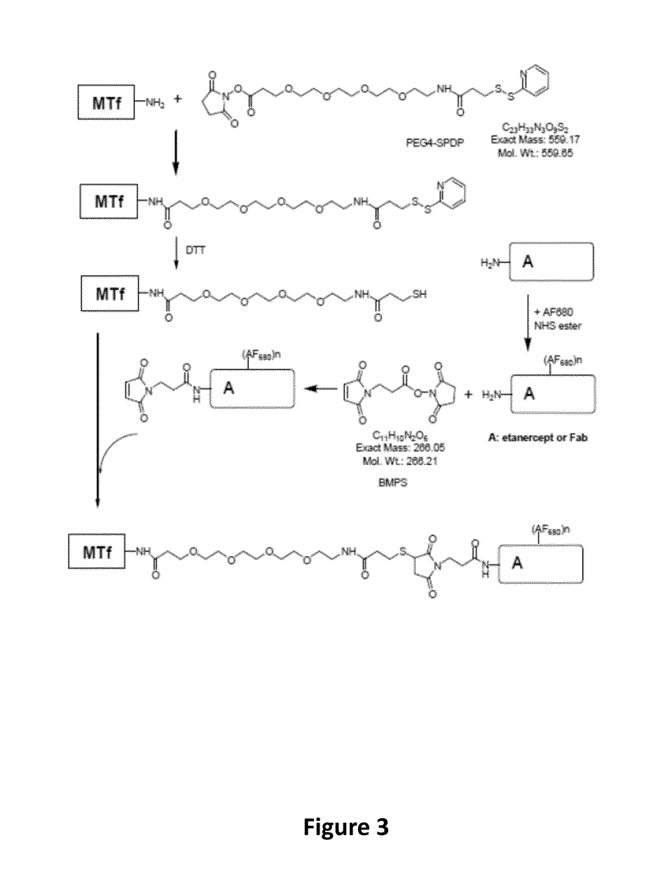

The following test proteins were prepared: AF680-labeled p97 melanotransferrin (MTf), AF680-labeled antibody Fab (Fab), AF680-labeled MTf-Fab conjugate (MTf-Fab), AF680-labeled etanercept (etanercept), and AF680-labeled MTf-etanercept conjugate (MTf-etanercept) The synthesis route of the test proteins is illustrated in FIG. 3.

Wild-type CD-1 albino mice were used to minimize endogenous autofluorescence in the brain. Therapeutic dose equivalents of each of the five test imaging test proteins were administered in 100 μl to mice via tail vein injection (intravenous injection). Prior to euthanasia, mice were injected (i.v.) with Tomato Lectin-FITC (40 μg) for 10 minutes to stain the brain vasculature. Transcardial perfusion was performed to remove the blood from the circulation, and brains were removed and cut into halves along the mid-coronal plane. Tissue blocks were covered in PCT and frozen in liquid nitrogen prior to storage at −80° C.

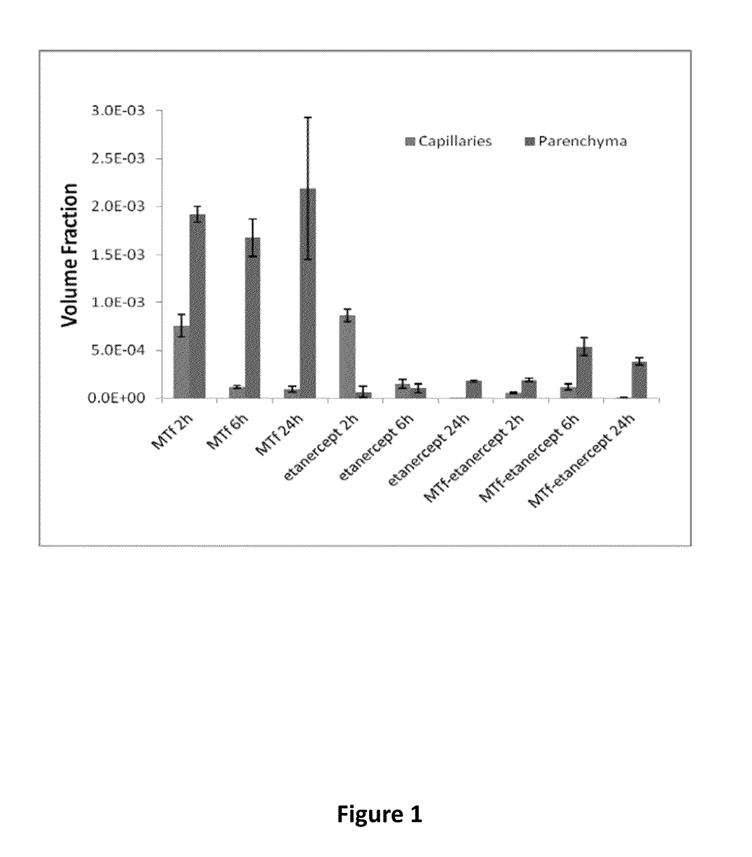

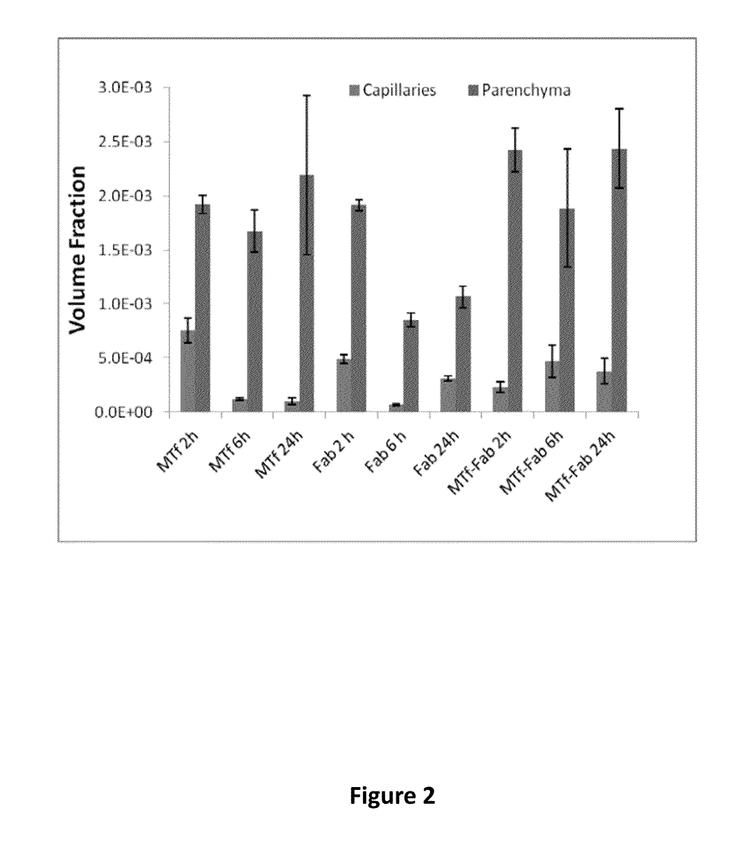

For confocal microscopy, confocal images of fluorescently labeled cells were acquired with an A Leica AOBS SP8 laser scanning confocal microscope (Leica, Heidelberg, Germany). The excitation wavelengths were at 405 (DAPI), 595 nm (Texas Red), and 670 nm (AF647), and an 80 MHz white light laser was used to collect the respective emission signals. All images and spectral data (except DAPI) were generated using highly sensitive HyD detectors. The backscattered emission signals from the sample were delivered through the tunable filter (AOBS). Three-dimensional (3D) image and volume fraction analysis was then performed. The results are shown in FIGS. 1 and 2.

FIG. 1 shows that conjugation of etanercept to MTf significantly increased the delivery of etanercept across the BBB and into parenchymal tissues of the brain. However, the levels of the MTf-etanercept conjugate in brain tissues were much lower than MTf alone, suggesting that etanercept contains at least one other binding motif that competes with/limits the activity of MTf in transporting the conjugate across the BBB. In contrast, FIG. 2 shows that conjugation of an anti-TNF/TNFR Fab to MTF not only significantly increased delivery of the Fab across the BBB and into parenchymal tissues of the brain, but also did so at levels comparable to MTf alone.

Because etanercept and the Fab are both directed at disrupting the TNF/TNFR ligand/receptor relationship, the main difference between the two is the presence of one or more Fc regions in the etanercept conjugate. These results suggest that the presence of an Fc region in an MTf conjugate can alter biodistribution of such conjugates, for example, by interacting with Fc receptor-expressing cells outside of the CNS, and thereby reduce transport across the BBB into parenchymal tissues of the brain. Thus, even though MTf is capable of significantly enhancing CNS delivery of proteins that contain an intact Fc region, the delivery of MTf conjugates across the BBB and into CNS tissues could be optimized/further enhanced by modifying the Fc region of a polypeptide of interest (e.g., therapeutic antibody, therapeutic Fc-fusion polypeptide such as etanercept) to reduce its binding to one or more Fc receptors/ligands.

1. A conjugate, comprising a blood-brain barrier (BBB)-transport moiety linked to a therapeutic antibody or a therapeutic Fc-fusion polypeptide, wherein the Fc region of the antibody or the Fc-fusion polypeptide is modified to reduce binding to one or more Fc receptors/ligands.

2. The conjugate of claim 1, wherein the antibody specifically binds to one or more of human Her2/neu, Her1/EGFR, TNF-α, B7H3 antigen, CD20, VEGF, CD52, CD33, CTLA-4, tenascin, alpha-4 (α4) integrin, IL-23, amyloid-β such as Aβ(1-42), Huntingtin, CD25, nerve growth factor (NGF), TrkA, or α-synuclein

3. The conjugate of claim 1, where the antibody specifically binds to a cancer-associated antigen.

4. The method of claim 3, where the cancer-associated antigen is one or more of human Her2/neu, Her1/EGF receptor (EGFR), Her3, A33 antigen, B7H3, CD5, CD19, CD20, CD22, CD23 (IgE Receptor), C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), or mesothelin.

5. The conjugate of claim 1, where the antibody specifically binds to a pro-inflammatory molecule, optionally a pro-inflammatory cytokine or chemokine.

6. The conjugate of claim 5, where the pro-inflammatory molecule is one or more of TNF-α, TNF-β, FasL, CD27L, CD30L, CD40L, Ox40L, 4-1BBL, TRAIL, TWEAK, and Apo3L, IL-1α, IL-1β, IL-2, interferon-γ (IFN-γ), IFN-α, IFN-β, IL-6, IL-8, IL-12, IL-15, IL-17, IL-18, IL-21, LIF, CCL5, GROα, MCP-1, MIP-1α, MIP-1β, macrophage colony stimulating factor (MCSF), or granulocyte macrophage colony stimulating factor (GM-CSF).

7. The conjugate of claim 6, where the pro-inflammatory molecule is TNF-α, and where the antibody is adalimumab, certolizumab pegol, golimumab, infliximab, D2E7, CDP 571, or CDP 870.

8. The conjugate of claim 1, where the antibody specifically binds to a pain-associated antigen.

9. The conjugate of claim 8, where the pain associated-antigen is one or more of nerve growth factor (NGF) or tropomyosin-related kinase A (TrkA).

10. The conjugate of claim 1, where the therapeutic antibody is selected from trastuzumab, cetuximab, rituximab, daclizumab, tanezumab, 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, cantuzumab (mertansine), cantuzumab (ravtansine), capromab (pendetide), catumaxomab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, tucotuzumab (celmoleukin), ublituximab, urelumab, veltuzumab, volociximab, votumumab, and zalutumumab.

11. (canceled)

12. The conjugate of claim 1, where the Fc-fusion polypeptide is selected from etanercept, abatercept, aflibercept, alefacept, belatacept, rilonacept, and romiplastin.

13. The conjugate of claim 1, where the Fc region is modified to reducing binding to one or more of a protein of the complement system, Fcγ receptors (FcγR), Fcα receptors (FcαR), Fcε receptors (FcεR), or the neonatal Fc receptor (FcRn), relative to a corresponding unmodified Fc region.

14. The conjugate of claim 13, where the protein of the complement system is C1, optionally the C1q subunit thereof.

15. The conjugate of claim 13, where the FcγR is selected from one or more of FcγRI, FcγRIIa, FcγRIIb, FcγRIIc, FcγRIIIa, and FcγRIIIb.

16. The conjugate of claim 13, where the FcαR is selected from one or both of FcαRI (CD89) or Fcα/μR.

17. The conjugate of claim 13, where the FcεR receptor is selected from one or both of FcεRI and FcεRII.

18. The conjugate of claim 1, where the Fc region is modified to reduce one or more effector functions selected from complement fixation or activation, complement-dependent cytotoxicity (CDC)-related activity, antibody-dependent cellular cytotoxicity (ADCC)-related activity, and/or antibody-dependent cell phagocytosis (ADCP)-related activity, relative to a corresponding unmodified Fc region.

19. The conjugate of claim 1, where the Fc region is modified by full or partial deletion of the Fc region, optionally including a full or partial deletion of the hinge region.

20. The conjugate of claim 19, where the Fc region is modified by full or partial deletion of one or more of the CH2 region, CH3 region, CH4 region, and/or hinge region.

21. The conjugate of claim 1, where the Fc region is modified by full or partial deletion of the C1q binding site.

22. The conjugate of claim 1, where the Fc region of the antibody is deleted to generate a Fab fragment or a F(ab′)2 fragment of the antibody.

23. The conjugate of claim 1, where the BBB-transport moiety is selected from one or more of a p97 (melanotransferrin) polypeptide, a Receptor Associated Protein (RAP), an aprotinin peptide or an analog thereof, a protein transduction domain (PTD), a human low-density lipoprotein receptor (hLDLR) binding peptide or an analog thereof, an antibody or natural ligand that binds to a BBB-associated receptor, and glutathione (GSH).

24. The conjugate of claim 23, where the p97 polypeptide comprises or consists of a sequence in Table B1, or an active variant or fragment thereof.

25. The conjugate of claim 24, where the p97 polypeptide is a soluble human p97 polypeptide.

26. The conjugate of claim 23, where the RAP comprises or consists of a sequence in Table B2, or an active variant or fragment thereof.

27. The conjugate of claim 23, where the aprotinin peptide comprises or consists of a sequence in Table B3, or an active variant or fragment thereof.

28. The conjugate of claim 23, where the PTD comprises or consists of a sequence in Table B4, or an active variant or fragment.

29. The conjugate of claim 23, where the hLDLR binding peptide comprises or consists of a sequence in Table B5, or an active variant or fragment.

30. The conjugate of claim 23, where the BBB-associated receptor is selected from one or more of the insulin receptor, the transferrin receptor, the leptin receptor, lipoprotein receptors such as the lipoprotein receptor-related protein (LRP-1) receptor, insulin-like growth factor (IGF) receptors such as IGF1R and IGF2R, the low-density lipoprotein receptor, the diptheria toxin receptor, and TMEM 30A (Flippase).

31. The conjugate of claim 23, where the BBB-associated receptor ligand is selected from one or more of insulin, transferrin and transferrin fragments, lactoferrin and lactoferrin fragments, apolipoprotein A (Apo A), apolipoprotein B (Apo B), apolipoprotein E (Apo E), and diptheria toxin (including non-toxic mutants thereof such as CRM45 and CRM197).

32. A composition, comprising a pharmaceutically acceptable carrier or excipient, and a conjugate of claim 1.

33. A method of treating a subject in need thereof, comprising administering to the subject a composition of claim 32.

34. The method of claim 33, for delivering a therapeutic antibody or Fc-fusion protein to the central nervous system of the subject in need thereof.

35. The method of claim 33, for treating a disease or condition selected from a cancer of the central nervous system (CNS), optionally the brain, a degenerative or autoimmune disorder of the CNS, pain, or an inflammatory condition.

36. The method of claim 35, for treating primary cancer of the CNS, optionally the brain.

37. The method of claim 35, for treating a metastatic cancer of the CNS, optionally the brain.

38-46. (canceled)

47. The method of claim 46, where the inflammatory condition has a central nervous system component.

48. (canceled)

49. The method of claim 47, where the inflammatory condition is associated with an infection of the central nervous system.

50-51. (canceled)

52. The method of claim 47, where the inflammatory condition is associated with a cancer of the CNS, optionally a malignant meningitis.


SEQUENCE LISTING (261 sequences)

SEQ ID NO: 1 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 2 - protein, 692 aa, Homo sapiens
GMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSG

SEQ ID NO: 3 - protein, 11 aa, Homo sapiens
WCATSDPEQHK

SEQ ID NO: 4 - protein, 11 aa, Homo sapiens
RSSHVTIDTLK

SEQ ID NO: 5 - protein, 13 aa, Homo sapiens
SSHVTIDTLKGVK

SEQ ID NO: 6 - protein, 14 aa, Homo sapiens
LCRGDSSGEGVCDK

SEQ ID NO: 7 - protein, 16 aa, Homo sapiens
GDSSGEGVCDKSPLER

SEQ ID NO: 8 - protein, 9 aa, Homo sapiens
YYDYSGAFR

SEQ ID NO: 9 - protein, 7 aa, Homo sapiens
ADVTEWR

SEQ ID NO: 10 - protein, 9 aa, Homo sapiens
VPAHAVVVR

SEQ ID NO: 11 - protein, 10 aa, Homo sapiens
ADTDGGLIFR

SEQ ID NO: 12 - protein, 9 aa, Homo sapiens
CGDMAVAFR

SEQ ID NO: 13 - protein, 11 aa, Homo sapiens
LKPEIQCVSAK

SEQ ID NO: 14 - protein, 12 aa, Homo sapiens
DSSHAFTLDELR

SEQ ID NO: 15 - protein, 13 aa, Homo sapiens
SEDYELLCPNGAR

SEQ ID NO: 16 - protein, 15 aa, Homo sapiens
AQDLFGDDHNKNGFK

SEQ ID NO: 17 - protein, 40 aa, Homo sapiens
FSSEAYGQKDLLFKDSTSELVPIATQTYEAWLGHEYLHAM

SEQ ID NO: 18 - protein, 221 aa, Homo sapiens
ERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVM

SEQ ID NO: 19 - protein, 32 aa, Homo sapiens
VRPDTNIFTVYGLLDKAQDLFGDDHNKNGFKM

SEQ ID NO: 20 - protein, 323 aa, Homo sapiens
YSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 21 - protein, 58 aa, Bos taurus [sequence omitted]

SEQ ID NO: 22 - protein, 19 aa, Bos taurus
TFVYGGCRAKRNNFKSAED

SEQ ID NO: 23 - protein, 19 aa, Artificial Sequence
  Aprotinin analogue peptide
TFFYGGCRGKRNNFKTEEY

SEQ ID NO: 24 - protein, 19 aa, Artificial Sequence
  Aprotinin analogue peptide
TFFYGGSRGKRNNFKTEEY

SEQ ID NO: 25 - protein, 19 aa, Artificial Sequence
  Aprotinin analogue peptide
TFVYGGCRAKRNNFKSAED

SEQ ID NO: 26 - protein, 19 aa, Artificial Sequence
  Aprotinin analogue peptide
TFQYGGCMGNGNNFVTEKE

SEQ ID NO: 27 - protein, 19 aa, Artificial Sequence
  Aprotinin analogue peptide
PFFYGGCGGNRNNFDTEEY

SEQ ID NO: 28 - protein, 19 aa, Artificial Sequence
  Aprotinin analogue peptide
SFYYGGCLGNKNNYLREEE

SEQ ID NO: 29 - protein, 19 aa, Artificial Sequence
  Aprotinin analogue peptide
TFFYGGCRAKRNNFKRAKY

SEQ ID NO: 30 - protein, 19 aa, Artificial Sequence
  Aprotinin analogue peptide
TFFYGGCRGKRNNFKRAKY

SEQ ID NO: 31 - protein, 19 aa, Artificial Sequence
  Aprotinin analogue peptide
TFFYGGCRAKKNNYKRAKY

SEQ ID NO: 32 - protein, 19 aa, Artificial Sequence
  Aprotinin analogue peptide
TFFYGGCRGKKNNFKRAKY

SEQ ID NO: 33 - protein, 19 aa, Artificial Sequence
  Aprotinin analogue peptide
TFQYGGCRAKRNNFKRAKY

SEQ ID NO: 34 - protein, 19 aa, Artificial Sequence
  Aprotinin analogue peptide
TFQYGGCRGKKNNFKRAKY

SEQ ID NO: 35 - protein, 19 aa, Artificial Sequence
  Aprotinin analogue peptide
TFFYGGCLGKRNNFKRAKY

SEQ ID NO: 36 - protein, 19 aa, Artificial Sequence
  Aprotinin analogue peptide
TFFYGGSLGKRNNFKRAKY

SEQ ID NO: 37 - protein, 19 aa, Artificial Sequence
  Aprotinin analogue peptide
PFFYGGCGGKKNNFKRAKY

SEQ ID NO: 38 - protein, 19 aa, Artificial Sequence
  Aprotinin analogue peptide
TFFYGGCRGKGNNYKRAKY

SEQ ID NO: 39 - protein, 19 aa, Artificial Sequence
  Aprotinin analogue peptide
PFFYGGCRGKRNNFLRAKY

SEQ ID NO: 40 - protein, 19 aa, Artificial Sequence
  Aprotinin analogue peptide
TFFYGGCRGKRNNFKREKY

SEQ ID NO: 41 - protein, 19 aa, Artificial Sequence
  Aprotinin analogue peptide
PFFYGGCRAKKNNFKRAKE

SEQ ID NO: 42 - protein, 19 aa, Artificial Sequence
  Aprotinin analogue peptide
TFFYGGCRGKRNNFKRAKD

SEQ ID NO: 43 - protein, 19 aa, Artificial Sequence
  Aprotinin analogue peptide
TFFYGGCRAKRNNFDRAKY

SEQ ID NO: 44 - protein, 19 aa, Artificial Sequence
  Aprotinin analogue peptide
TFFYGGCRGKKNNFKRAEY

SEQ ID NO: 45 - protein, 19 aa, Artificial Sequence
  Aprotinin analogue peptide
PFFYGGCGANRNNFKRAKY

SEQ ID NO: 46 - protein, 19 aa, Artificial Sequence
  Aprotinin analogue peptide
TFFYGGCGGKKNNFKTAKY

SEQ ID NO: 47 - protein, 19 aa, Artificial Sequence
  Aprotinin analogue peptide
TFFYGGCRGNRNNFLRAKY

SEQ ID NO: 48 - protein, 19 aa, Artificial Sequence
  Aprotinin analogue peptide
TFFYGGCRGNRNNFKTAKY

SEQ ID NO: 49 - protein, 19 aa, Artificial Sequence
  Aprotinin analogue peptide
TFFYGGSRGNRNNFKTAKY

SEQ ID NO: 50 - protein, 19 aa, Artificial Sequence
  Aprotinin analogue peptide
TFFYGGCLGNGNNFKRAKY

SEQ ID NO: 51 - protein, 19 aa, Artificial Sequence
  Aprotinin analogue peptide
TFFYGGCLGNRNNFLRAKY

SEQ ID NO: 52 - protein, 19 aa, Artificial Sequence
  Aprotinin analogue peptide
TFFYGGCLGNRNNFKTAKY

SEQ ID NO: 53 - protein, 19 aa, Artificial Sequence
  Aprotinin analogue peptide
TFFYGGCRGNGNNFKSAKY

SEQ ID NO: 54 - protein, 19 aa, Artificial Sequence
  Aprotinin analogue peptide
TFFYGGCRGKKNNFDREKY

SEQ ID NO: 55 - protein, 19 aa, Artificial Sequence
  Aprotinin analogue peptide
TFFYGGCRGKRNNFLREKE

SEQ ID NO: 56 - protein, 19 aa, Artificial Sequence
  Aprotinin analogue peptide
TFFYGGCRGKGNNFDRAKY

SEQ ID NO: 57 - protein, 19 aa, Artificial Sequence
  Aprotinin analogue peptide
TFFYGGSRGKGNNFDRAKY

SEQ ID NO: 58 - protein, 19 aa, Artificial Sequence
  Aprotinin analogue peptide
TFFYGGCRGNGNNFVTAKY

SEQ ID NO: 59 - protein, 19 aa, Artificial Sequence
  Aprotinin analogue peptide
PFFYGGCGGKGNNYVTAKY

SEQ ID NO: 60 - protein, 19 aa, Artificial Sequence
  Aprotinin analogue peptide
TFFYGGCLGKGNNFLTAKY

SEQ ID NO: 61 - protein, 19 aa, Artificial Sequence
  Aprotinin analogue peptide
SFFYGGCLGNKNNFLTAKY

SEQ ID NO: 62 - protein, 19 aa, Artificial Sequence
  Aprotinin analogue peptide
TFFYGGCGGNKNNFVREKY

SEQ ID NO: 63 - protein, 12 aa, Artificial Sequence
  Aprotinin analogue peptide
LGNKNNYVREKY

SEQ ID NO: 64 - protein, 19 aa, Artificial Sequence
  Aprotinin analogue peptide
TFFYGGCGGNGNNFLTAKY

SEQ ID NO: 65 - protein, 19 aa, Artificial Sequence
  Aprotinin analogue peptide
TFFYGGCRGNRNNFLTAEY

SEQ ID NO: 66 - protein, 19 aa, Artificial Sequence
  Aprotinin analogue peptide
TFFYGGCRGNGNNFKSAEY

SEQ ID NO: 67 - protein, 19 aa, Artificial Sequence
  Aprotinin analogue peptide
PFFYGGCLGNKNNFKTAEY

SEQ ID NO: 68 - protein, 19 aa, Artificial Sequence
  Aprotinin analogue peptide
TFFYGGCRGNRNNFKTEEY

SEQ ID NO: 69 - protein, 19 aa, Artificial Sequence
  Aprotinin analogue peptide
TFFYGGCRGKRNNFKTEED

SEQ ID NO: 70 - protein, 19 aa, Artificial Sequence
  Aprotinin analogue peptide
PFFYGGCGGNGNNFVREKY

SEQ ID NO: 71 - protein, 19 aa, Artificial Sequence
  Aprotinin analogue peptide
SFFYGGCMGNGNNFVREKY

SEQ ID NO: 72 - protein, 19 aa, Artificial Sequence
  Aprotinin analogue peptide
PFFYGGCGGNGNNFLREKY

SEQ ID NO: 73 - protein, 19 aa, Artificial Sequence
  Aprotinin analogue peptide
TFFYGGCLGNGNNFVREKY

SEQ ID NO: 74 - protein, 19 aa, Artificial Sequence
  Aprotinin analogue peptide
SFFYGGCLGNGNNYLREKY

SEQ ID NO: 75 - protein, 19 aa, Artificial Sequence
  Aprotinin analogue peptide
TFFYGGSLGNGNNFVREKY

SEQ ID NO: 76 - protein, 19 aa, Artificial Sequence
  Aprotinin analogue peptide
TFFYGGCRGNGNNFVTAEY

SEQ ID NO: 77 - protein, 19 aa, Artificial Sequence
  Aprotinin analogue peptide
TFFYGGCLGKGNNFVSAEY

SEQ ID NO: 78 - protein, 19 aa, Artificial Sequence
  Aprotinin analogue peptide
TFFYGGCLGNRNNFDRAEY

SEQ ID NO: 79 - protein, 19 aa, Artificial Sequence
  Aprotinin analogue peptide
TFFYGGCLGNRNNFLREEY

SEQ ID NO: 80 - protein, 19 aa, Artificial Sequence
  Aprotinin analogue peptide
TFFYGGCLGNKNNYLREEY

SEQ ID NO: 81 - protein, 19 aa, Artificial Sequence
  Aprotinin analogue peptide
PFFYGGCGGNRNNYLREEY

SEQ ID NO: 82 - protein, 19 aa, Artificial Sequence
  Aprotinin analogue peptide
PFFYGGSGGNRNNYLREEY

SEQ ID NO: 83 - protein, 19 aa, Artificial Sequence
  Aprotinin analogue peptide
MRPDFCLEPPYTGPCVARI

SEQ ID NO: 84 - protein, 21 aa, Artificial Sequence
  Aprotinin analogue peptide
ARIIRYFYNAKAGLCQTFVYG

SEQ ID NO: 85 - protein, 22 aa, Artificial Sequence
  Aprotinin analogue peptide
YGGCRAKRNNYKSAEDCMRTCG

SEQ ID NO: 86 - protein, 22 aa, Artificial Sequence
  Aprotinin analogue peptide
PDFCLEPPYTGPCVARIIRYFY

SEQ ID NO: 87 - protein, 19 aa, Artificial Sequence
  Aprotinin analogue peptide
TFFYGGCRGKRNNFKTEEY

SEQ ID NO: 88 - protein, 19 aa, Artificial Sequence
  Aprotinin analogue peptide
KFFYGGCRGKRNNFKTEEY

SEQ ID NO: 89 - protein, 19 aa, Artificial Sequence
  Aprotinin analogue peptide
TFYYGGCRGKRNNYKTEEY

SEQ ID NO: 90 - protein, 19 aa, Artificial Sequence
  Aprotinin analogue peptide
TFFYGGSRGKRNNFKTEEY

SEQ ID NO: 91 - protein, 20 aa, Artificial Sequence
  Aprotinin analogue peptide
CTFFYGCCRGKRNNFKTEEY

SEQ ID NO: 92 - protein, 20 aa, Artificial Sequence
  Aprotinin analogue peptide
TFFYGGCRGKRNNFKTEEYC

SEQ ID NO: 93 - protein, 19 aa, Artificial Sequence
  Aprotinin analogue peptide
TFFYGGKRGKRNNFKTEEY

SEQ ID NO: 94 - protein, 19 aa, Artificial Sequence
  Aprotinin analogue peptide
TFFYGGCRGKRNNFKTKRY

SEQ ID NO: 95 - protein, 19 aa, Artificial Sequence
  Aprotinin analogue peptide
TFFYGGKRGKRNNFKTAEY

SEQ ID NO: 96 - protein, 19 aa, Artificial Sequence
  Aprotinin analogue peptide
TFFYGGKRGKRNNFKRAGY

SEQ ID NO: 97 - protein, 19 aa, Artificial Sequence
  Aprotinin analogue peptide
TFFYGGKRGKRNNFKREKY

SEQ ID NO: 98 - protein, 19 aa, Artificial Sequence
  Aprotinin analogue peptide
TFFYGGKRGKRNNFKTAKY

SEQ ID NO: 99 - protein, 19 aa, Artificial Sequence
  Aprotinin analogue peptide
TFFYGGCLGNRNNFKTEEY

SEQ ID NO: 100 - protein, 19 aa, Artificial Sequence
  Aprotinin analogue peptide
TFFYGCGRGKRNNFKTEEY

SEQ ID NO: 101 - protein, 19 aa, Artificial Sequence
  Aprotinin analogue peptide
TFFYGGRCGKRNNFKTEEY

SEQ ID NO: 102 - protein, 20 aa, Artificial Sequence
  Aprotinin analogue peptide
TFFYGGCLGNGNNFDTEEEQ

SEQ ID NO: 103 - protein, 19 aa, Artificial Sequence
  Aprotinin analogue peptide
TFQYGGCRGKRNNFKTEEY

SEQ ID NO: 104 - protein, 19 aa, Artificial Sequence
  Aprotinin analogue peptide
YNKEFGTFNTKGCERGYRF

SEQ ID NO: 105 - protein, 19 aa, Artificial Sequence
  Aprotinin analogue peptide
RFKYGGCLGNMNNFETLEE

SEQ ID NO: 106 - protein, 19 aa, Artificial Sequence
  Aprotinin analogue peptide
RFKYGGCLGNKNNFLRLKY

SEQ ID NO: 107 - protein, 19 aa, Artificial Sequence
  Aprotinin analogue peptide
RFKYGGCLGNKNNYLRLKY

SEQ ID NO: 108 - protein, 22 aa, Artificial Sequence
  Aprotinin analogue peptide
KTKRKRKKQRVKIAYEEIFKNY

SEQ ID NO: 109 - protein, 15 aa, Artificial Sequence
  Aprotinin analogue peptide
KTKRKRKKQRVKIAY

SEQ ID NO: 110 - protein, 17 aa, Artificial Sequence
  Aprotinin analogue peptide
RGGRLSYSRRFSTSTGR

SEQ ID NO: 111 - protein, 10 aa, Artificial Sequence
  Aprotinin analogue peptide
RRLSYSRRRF

SEQ ID NO: 112 - protein, 16 aa, Artificial Sequence
  Aprotinin analogue peptide
RQIKIWFQNRRMKWKK

SEQ ID NO: 113 - protein, 19 aa, Artificial Sequence
  Aprotinin analogue peptide
TFFYGGSRGKRNNFKTEEY

SEQ ID NO: 114 - protein, 59 aa, Artificial Sequence
  Aprotinin analogue peptide
MRPDFCLEPPYTGPCVARIIRYFYNAKAGLCQTFVYGGCRAKRNNFKSAEDCMRTCGGA

SEQ ID NO: 115 - protein, 19 aa, Artificial Sequence
  Aprotinin analogue peptide
TFFYGGCRGKRNNFKTKEY

SEQ ID NO: 116 - protein, 19 aa, Artificial Sequence
  Aprotinin analogue peptide
TFFYGGCRAKRNNFKRAKY

SEQ ID NO: 117 - protein, 35 aa, Artificial Sequence
  Aprotinin analogue peptide
NAKAGLCQTFVYGGCLAKRNNFESAEDCMRTCGGA

SEQ ID NO: 118 - protein, 24 aa, Artificial Sequence
  Aprotinin analogue peptide
YGGCRAKRNNFKSAEDCMRTCGGA

SEQ ID NO: 119 - protein, 22 aa, Artificial Sequence
  Aprotinin analogue peptide
GLCQTFVYGGCRAKRNNFKSAE

SEQ ID NO: 120 - protein, 20 aa, Artificial Sequence
  Aprotinin analogue peptide
LCQTFVYGGCEAKRNNFKSA

SEQ ID NO: 121 - protein, 19 aa, Artificial Sequence
  Aprotinin analogue peptide
TFFYGGSRGKRNNFKTEEY

SEQ ID NO: 122 - protein, 19 aa, Artificial Sequence
  Aprotinin analogue peptide
RFFYGGSRGKRNNFKTEEY

SEQ ID NO: 123 - protein, 19 aa, Artificial Sequence
  Aprotinin analogue peptide
RFFYGGSRGKRNNFKTEEY

SEQ ID NO: 124 - protein, 19 aa, Artificial Sequence
  Aprotinin analogue peptide
RFFYGGSRGKRNNFRTEEY

SEQ ID NO: 125 - protein, 19 aa, Artificial Sequence
  Aprotinin analogue peptide
TFFYGGSRGKRNNFRTEEY

SEQ ID NO: 126 - protein, 19 aa, Artificial Sequence
  Aprotinin analogue peptide
TFFYGGSRGRRNNFRTEEY

SEQ ID NO: 127 - protein, 20 aa, Artificial Sequence
  Aprotinin analogue peptide
CTFFYGGSRGKRNNFKTEEY

SEQ ID NO: 128 - protein, 20 aa, Artificial Sequence
  Aprotinin analogue peptide
TFFYGGSRGKRNNFKTEEYC

SEQ ID NO: 129 - protein, 20 aa, Artificial Sequence
  Aprotinin analogue peptide
CTFFYGGSRGRRNNFRTEEY

SEQ ID NO: 130 - protein, 20 aa, Artificial Sequence
  Aprotinin analogue peptide
TFFYGGSRGRRNNFRTEEYC

SEQ ID NO: 131 - protein, 86 aa, Human immunodeficiency virus-1
MEPVDPRLEPWKHPGSQPKTACTTCYCKKCCFHCQVCFTTKALGISYGRKKRRQRRRPPQGSQTHQVSLSKQPTSQPRGDPTGPKE

SEQ ID NO: 132 - protein, 20 aa, Human immunodeficiency virus-1
FTTKALGISYGRKKRRQRRR

SEQ ID NO: 133 - protein, 16 aa, Drosiphila melanogaster
RQIKIWFQNRRMKWKK

SEQ ID NO: 134 - protein, 18 aa, Sus scrofa
RGGRLCYCRRRFCVCVGR

SEQ ID NO: 135 - protein, 19 aa, Artificial Sequence
  PG-1 analogue peptide (SynB1)
RGGRLSYSRRRFSTSTGRA

SEQ ID NO: 136 - protein, 10 aa, Artificial Sequence
  PG-1 analogue peptide (SynB3)
RRLSYSRRRF

SEQ ID NO: 137 - protein, 17 aa, Artificial Sequence
  PG-1 analogue peptide (SynB5)
RGGRLAYLRRRWAVLGR

SEQ ID NO: 138 - protein, 12 aa, artificial sequence
  synthesized peptide
HLDCMPRGCFRN

SEQ ID NO: 139 - protein, 10 aa, artificial sequence
  synthesized peptide
ACQVKSMPRC

SEQ ID NO: 140 - protein, 10 aa, artificial sequence
  synthesized peptide
ACTTPMPRLC

SEQ ID NO: 141 - protein, 10 aa, artificial sequence
  synthesized peptide
ACKAPQMPRC

SEQ ID NO: 142 - protein, 10 aa, artificial sequence
  synthesized peptide
ACLNPSMPRC

SEQ ID NO: 143 - protein, 10 aa, artificial sequence
  synthesized peptide
ACLVSSMPRC

SEQ ID NO: 144 - protein, 10 aa, artificial sequence
  synthesized peptide
ACLQPMPRLC

SEQ ID NO: 145 - protein, 10 aa, artificial sequence
  synthesized peptide
ACPVSSMPRC

SEQ ID NO: 146 - protein, 10 aa, artificial sequence
  synthesized peptide
ACQSPMPRLC

SEQ ID NO: 147 - protein, 10 aa, artificial sequence
  synthesized peptide
ACLTPMPRLC

SEQ ID NO: 148 - protein, 15 aa, artificial sequence
  synthesized peptide
DSGLCMPRLRGCDPR

SEQ ID NO: 149 - protein, 10 aa, artificial sequence
  synthesized peptide
ACMPRLRGCA

SEQ ID NO: 150 - protein, 15 aa, artificial sequence
  synthesized peptide
ASGLCMPRLRGCDPR

SEQ ID NO: 151 - protein, 15 aa, artificial sequence
  synthesized peptide
DAGLCMPRLRGCDPR

SEQ ID NO: 152 - protein, 15 aa, artificial sequence
  synthesized peptide
DSALCMPRLRGCDPR

SEQ ID NO: 153 - protein, 15 aa, artificial sequence
  synthesized peptide
DSGACMPRLRGCDPR

SEQ ID NO: 154 - protein, 15 aa, artificial sequence
  synthesized peptide
DSGLCMPRARGCDPR

SEQ ID NO: 155 - protein, 15 aa, artificial sequence
  synthesized peptide
DSGLCMPRLAGCDPR

SEQ ID NO: 156 - protein, 15 aa, artificial sequence
  synthesized peptide
DSGLCMPRLRACDPR

SEQ ID NO: 157 - protein, 15 aa, artificial sequence
  synthesized peptide
DSGLCMPRLRGCAPR

SEQ ID NO: 158 - protein, 15 aa, artificial sequence
  synthesized peptide
DSGLCMPRLRGCDAR

SEQ ID NO: 159 - protein, 15 aa, artificial sequence
  synthesized peptide
DSGLCMPRLRGCDPA

SEQ ID NO: 160 - protein, 14 aa, artificial sequence
  synthesized peptide
SGLCMPRLRGCDPR

SEQ ID NO: 161 - protein, 13 aa, artificial sequence
  synthesized peptide
GLCMPRLRGCDPR

SEQ ID NO: 162 - protein, 8 aa, artificial sequence
  synthesized peptide
CMPRLRGC

SEQ ID NO: 163 - protein, 8 aa, artificial sequence
  synthesized peptide
CMPRARGC

SEQ ID NO: 164 - protein, 8 aa, artificial sequence
  synthesized peptide
CMPRLAGC

SEQ ID NO: 165 - protein, 8 aa, artificial sequence
  synthesized peptide
CMPRLRAC

SEQ ID NO: 166 - protein, 8 aa, artificial sequence
  synthesized peptide
CMPRLKGC

SEQ ID NO: 167 - protein, 8 aa, artificial sequence
  synthesized peptide
CMPRLRGC

SEQ ID NO: 168 - protein, 8 aa, artificial sequence
  synthesized peptide
CMPRLRGC

SEQ ID NO: 169 - protein, 8 aa, artificial sequence
  synthesized peptide
CMPRLRGC

SEQ ID NO: 170 - protein, 8 aa, artificial sequence
  synthesized peptide
CMPRLRGC

SEQ ID NO: 171 - protein, 8 aa, artificial sequence
  synthesized peptide
CMPRLRGC

SEQ ID NO: 172 - protein, 8 aa, artificial sequence
  synthesized peptide
CMPRLRXC

SEQ ID NO: 173 - protein, 8 aa, artificial sequence
  synthesized peptide
CMPRKRGC

SEQ ID NO: 174 - protein, 7 aa, artificial sequence
  synthesized peptide
CMPRLRC

SEQ ID NO: 175 - protein, 7 aa, artificial sequence
  synthesized peptide
CMPRLCR

SEQ ID NO: 176 - protein, 8 aa, artificial sequence
  synthesized peptide
CMPRCLRG

SEQ ID NO: 177 - protein, 6 aa, artificial sequence
  synthesized peptide
CMPRGC

SEQ ID NO: 178 - protein, 8 aa, artificial sequence
  synthesized peptide
XMPRLRGC

SEQ ID NO: 179 - protein, 8 aa, artificial sequence
  synthesized peptide
XMPRLRGC

SEQ ID NO: 180 - protein, 8 aa, artificial sequence
  synthesized peptide
CMPRLRGX

SEQ ID NO: 181 - protein, 8 aa, artificial sequence
  synthesized peptide
XMPRLRGX

SEQ ID NO: 182 - protein, 8 aa, artificial sequence
  synthesized peptide
XMPRLRGX

SEQ ID NO: 183 - protein, 8 aa, artificial sequence
  synthesized peptide
XMPRLRGX

SEQ ID NO: 184 - protein, 8 aa, artificial sequence
  synthesized peptide
XMPRLRGC

SEQ ID NO: 185 - protein, 15 aa, artificial sequence
  synthesized peptide
MTVMPTGLWNPLIPS

SEQ ID NO: 186 - protein, 15 aa, artificial sequence
  synthesized peptide
SASWFAVPIPPLRLE

SEQ ID NO: 187 - protein, 15 aa, artificial sequence
  synthesized peptide
MTPMSTPRMLPVYVA

SEQ ID NO: 188 - protein, 15 aa, artificial sequence
  synthesized peptide
MTATHLSTLFQPLTY

SEQ ID NO: 189 - protein, 15 aa, artificial sequence
  synthesized peptide
MSPIPPAASTWANTL

SEQ ID NO: 190 - protein, 15 aa, artificial sequence
  synthesized peptide
MTANPLQNAPGPLSL

SEQ ID NO: 191 - protein, 15 aa, artificial sequence
  synthesized peptide
MQTAPPPPLTRVQWS

SEQ ID NO: 192 - protein, 15 aa, artificial sequence
  synthesized peptide
GTPRMHIPLNVDHLP

SEQ ID NO: 193 - protein, 15 aa, artificial sequence
  synthesized peptide
LTLPPISGLSSYPLP

SEQ ID NO: 194 - protein, 15 aa, artificial sequence
  synthesized peptide
TPSAHAMALQSLSVG

SEQ ID NO: 195 - protein, 15 aa, artificial sequence
  synthesized peptide
LTLPPISGLSSYPLP

SEQ ID NO: 196 - protein, 15 aa, artificial sequence
  synthesized peptide
MGTLNAPTAYPQDSL

SEQ ID NO: 197 - protein, 15 aa, artificial sequence
  synthesized peptide
LTNPPAYLPQNTDPH

SEQ ID NO: 198 - protein, 15 aa, artificial sequence
  synthesized peptide
MGLPLPYIQTILHTP

SEQ ID NO: 199 - protein, 15 aa, artificial sequence
  synthesized peptide
SAALIAMSSFKSITA

SEQ ID NO: 200 - protein, 15 aa, artificial sequence
  synthesized peptide
SGFAFARSVPTESRR

SEQ ID NO: 201 - protein, 15 aa, artificial sequence
  synthesized peptide
MTSPYMSLPPSTDDM

SEQ ID NO: 202 - protein, 15 aa, artificial sequence
  synthesized peptide
LTNPPAYLPQNTDPH

SEQ ID NO: 203 - protein, 8 aa, Artificial Sequence
  hLDLR binding peptide motif
XMXRLXXX

SEQ ID NO: 204 - protein, 8 aa, Artificial Sequence
  hLDLR binding peptide motif
XMXRLRGC

SEQ ID NO: 205 - protein, 8 aa, Artificial sequence
  synthesized
CMXRLRGC

SEQ ID NO: 206 - protein, 8 aa, Artificial sequence
  synthesized
XMXRLRGC

SEQ ID NO: 207 - protein, 8 aa, Artificial sequence
  synthesized
XMXRLRGC

SEQ ID NO: 208 - protein, 8 aa, Artificial sequence
  synthesized
CMXRLRGC

SEQ ID NO: 209 - protein, 8 aa, Artificial sequence
  synthesized
XMXRLRGC

SEQ ID NO: 210 - protein, 8 aa, Artificial sequence
  synthesized
XMXRLRGC

SEQ ID NO: 211 - protein, 8 aa, Artificial sequence
  synthesized
CMXRLRXC

SEQ ID NO: 212 - protein, 8 aa, Artificial sequence
  synthesized
CMXRLRGX

SEQ ID NO: 213 - protein, 8 aa, Artificial sequence
  synthesized
CMXRLRXX

SEQ ID NO: 214 - protein, 8 aa, Artificial sequence
  synthesized
CMXRLRXC

SEQ ID NO: 215 - protein, 8 aa, Artificial sequence
  synthesized
CMXRLRGX

SEQ ID NO: 216 - protein, 8 aa, Artificial sequence
  synthesized
CMXRLRXX

SEQ ID NO: 217 - protein, 8 aa, Artificial sequence
  synthesized
CMPXLRGC

SEQ ID NO: 218 - protein, 8 aa, Artificial sequence
  synthesized
CMPXLRGC

SEQ ID NO: 219 - protein, 8 aa, Artificial sequence
  synthesized
CMPXLRGC

SEQ ID NO: 220 - protein, 8 aa, Artificial sequence
  synthesized
CMPXLRGC

SEQ ID NO: 221 - protein, 8 aa, Artificial sequence
  synthesized
CMPRLXGC

SEQ ID NO: 222 - protein, 8 aa, Artificial sequence
  synthesized
CMPRLXGC

SEQ ID NO: 223 - protein, 8 aa, Artificial sequence
  synthesized
CMPRLRGC

SEQ ID NO: 224 - protein, 8 aa, Artificial sequence
  synthesized
CMPRLRGC

SEQ ID NO: 225 - protein, 230 aa, Artificial Sequence
  Synthetic Fc Construct
XESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 226 - protein, 5 aa, Artificial Sequence
  Sulfatase motif
XXXXX

SEQ ID NO: 227 - protein, 5 aa, Artificial Sequence
  Sulfatase motifs
XGXXX

SEQ ID NO: 228 - protein, 4 aa, Artificial Sequence
  Exemplary Gly Ser linker
GSGS

SEQ ID NO: 229 - protein, 4 aa, Artificial Sequence
  Exemplary Gly Ser linker
GGSG

SEQ ID NO: 230 - protein, 4 aa, Artificial Sequence
  Exemplary Gly Ser linker
GGGS

SEQ ID NO: 231 - protein, 5 aa, Artificial Sequence
  Exemplary Gly Ser linker
GGGGS

SEQ ID NO: 232 - protein, 4 aa, Artificial Sequence
  Exemplary Gly Asn linker
GNGN

SEQ ID NO: 233 - protein, 4 aa, Artificial Sequence
  Exemplary Gly Asn linker
GGNG

SEQ ID NO: 234 - protein, 4 aa, Artificial Sequence
  Exemplary Gly Asn linker
GGGN

SEQ ID NO: 235 - protein, 5 aa, Artificial Sequence
  Exemplary Gly Asn linker
GGGGN

SEQ ID NO: 236 - protein, 4 aa, Artificial Sequence
  Enzymatically degradable linker - thrombin cleavable
GRGD

SEQ ID NO: 237 - protein, 6 aa, Artificial Sequence
  Enzymatically degradable linker - thrombin cleavable
GRGDNP

SEQ ID NO: 238 - protein, 5 aa, Artificial Sequence
  Enzymatically degradable linker - thrombin cleavable
GRGDS

SEQ ID NO: 239 - protein, 7 aa, Artificial Sequence
  Enzymatically degradable linker - thrombin cleavable
GRGDSPK

SEQ ID NO: 240 - protein, 4 aa, Artificial Sequence
  Enzymatically degradable linker - elastase cleavable
AAPV

SEQ ID NO: 241 - protein, 4 aa, Artificial Sequence
  Enzymatically degradable linker - elastase cleavable
AAPL

SEQ ID NO: 242 - protein, 4 aa, Artificial Sequence
  Enzymatically degradable linker - elastase cleavable
AAPF

SEQ ID NO: 243 - protein, 4 aa, Artificial Sequence
  Enzymatically degradable linker - elastase cleavable
AAPA

SEQ ID NO: 244 - protein, 4 aa, Artificial Sequence
  Enzymatically degradable linker - elastase cleavable
AYLV

SEQ ID NO: 245 - protein, 6 aa, Artificial Sequence
  Enzymatically degradable linker - matrix metalloproteinase cleavable
GPXGPX

SEQ ID NO: 246 - protein, 6 aa, Artificial Sequence
  Enzymatically degradable linker - matrix metalloproteinase cleavable
GPLGPX

SEQ ID NO: 247 - protein, 6 aa, Artificial Sequence
  Enzymatically degradable linker - matrix metalloproteinase cleavable
GPIGPX

SEQ ID NO: 248 - protein, 5 aa, Artificial Sequence
  Enzymatically degradable linker - matrix metalloproteinase cleavable
APGLX

SEQ ID NO: 249 - protein, 7 aa, Artificial Sequence
  Enzymatically degradable linker - collagenase cleavable
PLGPDRX

SEQ ID NO: 250 - protein, 7 aa, Artificial Sequence
  Enzymatically degradable linker - collagenase cleavable
PLGLLGX

SEQ ID NO: 251 - protein, 7 aa, Artificial Sequence
  Enzymatically degradable linker - collagenase cleavable
PQGIAGW

SEQ ID NO: 252 - protein, 5 aa, Artificial Sequence
  Enzymatically degradable linker - collagenase cleavable
PLGCH

SEQ ID NO: 253 - protein, 6 aa, Artificial Sequence
  Enzymatically degradable linker - collagenase cleavable
PLGLYA

SEQ ID NO: 254 - protein, 7 aa, Artificial Sequence
  Enzymatically degradable linker - collagenase cleavable
PLALWAR

SEQ ID NO: 255 - protein, 7 aa, Artificial Sequence
  Enzymatically degradable linker - collagenase cleavable
PLAYWAR

SEQ ID NO: 256 - protein, 7 aa, Artificial Sequence
  Enzymatically degradable linker - stromelysin cleavable
PYAYYMR

SEQ ID NO: 257 - protein, 7 aa, Artificial Sequence
  Enzymatically degradable linker - gelatinase cleavable
PLGMYSR

SEQ ID NO: 258 - protein, 4 aa, Artificial Sequence
  Enzymatically degradable linker - angiotensin converting enzyme cleavable
GDKP

SEQ ID NO: 259 - protein, 5 aa, Artificial Sequence
  Enzymatically degradable linker - angiotensin converting enzyme cleavable
GSDKP

SEQ ID NO: 260 - protein, 4 aa, Artificial Sequence
  Enzymatically degradable linker - cathepsin B cleavable
ALAL

SEQ ID NO: 261 - protein, 4 aa, Artificial Sequence
  Enzymatically degradable linker - cathepsin B cleavable
GFLG